(19) United States
(12) Patent Application Publication
(10) Pub. No.: US 2023/0033360 A1
(43) Pub. Date: Feb. 2, 2023

(54) COMPOUNDS AS CD73 INHIBITORS

(71) Applicant: BioArdis LLC, San Diego, CA (US)

(72) Inventors: Jianxia FENG, Beijing (CN); Dianjun CHEN, Beijing (CN); Frank KAYSER, San Rafael, CA (US); Chong LIU, Beijing (CN)

(21) Appl. No.: 17/770,952

(22) PCT Filed: Nov. 5, 2020
(86) PCT No.: PCT/CN2020/126631

§ 371 Date: Apr. 21, 2022

(30) Foreign Application Priority Data
Nov. 5, 2019 (CN) PCT/CN2019/115702
Oct. 19, 2020 (CN) PCT/CN2020/121863

(51) Int. Cl. A61K 31/675; A61K 45/06; C07F 9/6561

(52) U.S. Cl. CPC A61K 31/675; A61K 45/06; C07F 9/6561

(57) ABSTRACT

Provided herein are the compounds that are inhibitors of CD73 and are useful in treating CD73-associated diseases or conditions. Compositions containing the compounds are also provided.

### CROSS REFERENCE TO RELATED APPLICATIONS

This application claims priority to International Patent Application No. PCT/CN2019/115702, filed on Nov. 5, 2019, and International Patent Application No. PCT/CN2020/121863, filed on Oct. 19, 2020, the content of each of which is incorporated herein by reference in its entirety.

### FIELD

The present disclosure relates generally to compounds that are inhibitors of CD73 and are useful in treating CD73-associated diseases or conditions. Compositions containing the compounds of the present disclosure are also provided.

### BACKGROUND

CD73 is a 70-kDa glycosylphosphatidylinositol (GPI)-anchored protein normally expressed on endothelial cells and subsets of hematopoietic cells. CD73 is up-regulated by hypoxia-inducible factor (HIF)-1α and after exposure to type I interferons. In steady state, CD73 regulates vascular barrier function, restricts lymphocyte migration to draining lymph nodes, and stimulates mucosal hydration.

CD73 expression on tumor cells has been reported in several types of cancer, including bladder cancer, leukemia, glioma, glioblastoma, melanoma, ovarian cancer, thyroid cancer, esophageal cancer, prostate cancer, and breast cancer. (Stagg, et al., Proc. Natl. Acad. Sci. USA 107(4): 1547-1552). Notably, CD73 expression has been associated with a prometastatic phenotype in melanoma and breast cancer.

There is still a need for new CD73 inhibitors. In this regard, the compounds provided herein address the need.

### BRIEF SUMMARY

In one aspect, provided herein is a compound of formula (I):

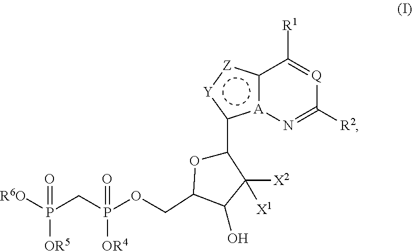

or a stereoisomer, tautomer, or a pharmaceutically acceptable salt of any of the foregoing, wherein

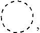

A, Z, Y, Q, X¹, X², and R¹, R², and R⁴-R⁵ are as described herein.

In another aspect, provided herein is a composition comprising a compound of formula (I), or a stereoisomer, tautomer or a pharmaceutically acceptable salt of any of the foregoing and a pharmaceutically acceptable excipient.

In another aspect, provided herein is a kit comprising a compound of formula (I), or a stereoisomer, tautomer, prodrug, or a pharmaceutically acceptable salt of any of the foregoing. In some embodiments, provided herein is a medicament comprising a compound of formula (I), or a stereoisomer, tautomer, prodrug, or a pharmaceutically acceptable salt of any of the foregoing.

In another aspect, provided herein is a method of treating a treating a disease mediated by CD73 in an individual in need thereof, comprising administering to the individual a therapeutically effective amount of a compound of formula (I), or a stereoisomer, tautomer, prodrug, or a pharmaceutically acceptable salt of any of the foregoing. In some embodiments, the disease is cancer. In some embodiments, the disease is bladder cancer, leukemia, glioma, glioblastoma, melanoma, ovarian cancer, thyroid cancer, esophageal cancer, prostate cancer, lung cancer, colorectal cancer, pancreatic cancer, skin cancer, liver cancer, gastric cancer, head & neck cancer, or breast cancer. In some embodiments, the method further comprises administering to the individual an additional therapeutic agent, wherein the additional therapeutic agent is an immune checkpoint inhibitor, a chemotherapeutic agent, an immune-modulating agent, an inflammation-modulating agent, or an anti-infective agent. In some embodiments, the additional therapeutic agent is an immune checkpoint inhibitor. In some embodiments, the additional therapeutic agent is a cytotoxic T lymphocyte associated protein 4 (CTLA-4) inhibitor, a programmed cell death protein 1 (PD-1) inhibitor, or a programmed death ligand 1 (PD-L1) inhibitor.

In another aspect, provided is a method of reversing or stopping the progression of CD73-mediated immunosuppression in an individual, comprising administering to the individual a therapeutically effective amount of a compound of formula (I), or a stereoisomer, tautomer, prodrug, or a pharmaceutically acceptable salt of any of the foregoing.

In another aspect, provided herein is a method of inhibition CD73, comprising contacting CD73 with a compound of formula (I), or a stereoisomer, tautomer, prodrug, or a pharmaceutically acceptable salt of any of the foregoing. In some embodiments, provided is a method of inhibiting CD73-catalyzed hydrolysis of adenosine monophosphate, comprising contacting CD73 with a compound of formula (I), or a stereoisomer, tautomer, prodrug, or a pharmaceutically acceptable salt of any of the foregoing.

In another aspect, provided herein are methods of preparing a compound of formula (I), or a stereoisomer, tautomer, prodrug, or a pharmaceutically acceptable salt of any of the foregoing, according to the procedures detailed herein.

### DETAILED DESCRIPTION

Described herein are compounds, including therapeutic agents, that can inhibit CD73. These compounds can be used in the prevention and/or treatment of certain pathological conditions as described herein.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having 1 to 10 carbon atoms (a “C₁₋₁₀ alkyl”), having 6 to 10 carbon atoms (a “C₆₋₁₀ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkoxy” refers to an —O-alkyl. Examples of alkoxy include, but are not limited to, methoxy, ethoxy, n-propoxy, isopropoxy, n-butoxy, iso-butoxy, sec-butoxy, and tert-butoxy.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂₋₁₀ means two to ten carbon atoms). An alkenyl group may have “cis” or “trans” configurations, or alternatively have “E” or “Z” configurations. Particular alkenyl groups are those having 2 to 20 carbon atoms (a “C₂₋₂₀ alkenyl”), having 6 to 10 carbon atoms (a “C₆₋₁₀ alkenyl”), having 2 to 8 carbon atoms (a “C₂₋ₛ alkenyl”), having 2 to 6 carbon atoms (a “C₂₋₆ alkenyl”), or having 2 to 4 carbon atoms (a “C₂₋₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C═C) and having the number of carbon atoms designated (i.e., C₂₋₁₀ means two to ten carbon atoms). Particular alkynyl groups are those having 2 to 20 carbon atoms (a “C₂₋₂₀ alkynyl”), having 6 to 10 carbon atoms (a “C₆₋₁₀ alkynyl”), having 2 to 8 carbon atoms (a “C₂₋₈ alkynyl”), having 2 to 6 carbon atoms (a “C₂₋₆ alkynyl”), or having 2 to 4 carbon atoms (a “C₂₋₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Cycloalkyl” as used herein refers to and includes, unless otherwise stated, cyclic univalent nonaromatic hydrocarbon structures, which may be fully saturated, mono- or polyunsaturated, but which are non-aromatic, having the number of carbon atoms designated (i.e., C₃₋₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃₋₈ cycloalkyl”), having 3 to 6 carbon atoms (a “C₃₋₆ cycloalkyl”), or having from 3 to 4 annular carbon atoms (a “C₃₋₄ cycloalkyl”). Examples of cycloalkyl include, but are not limited to, cyclopropyl, cyclobutyl, cyclopentyl, cyclohexyl, cycloheptyl, norbornyl, and the like. A cycloalkyl group may be fused with aryl, heteroaryl, or heterocyclyl. In one variation, a cycloalkyl group having more than one ring where at least one ring is aryl, heteroaryl, or heterocyclyl is connected to the parent structure at an annular atom in the nonaromatic hydrocarbon cyclic group.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆₋₁₄ aryl”). An aryl group may be fused with heteroaryl, cycloalkyl, or heterocyclyl. In one variation, an aryl group having more than one ring where at least one ring is heteroaryl, cycloalkyl, or heterocyclyl is connected to the parent structure at an annular atom in the aromatic carbocyclic group.

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group may be fused with aryl, cycloalkyl, or heterocyclyl. In one variation, a heteroaryl group having more than one ring where at least one ring is aryl, cycloalkyl, or heterocyclyl is connected to the parent structure at an annular atom in the aromatic cyclic group having at least one annular heteroatom. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur. A heterocyclyl group may be fused with aryl, cycloalkyl, or heteroaryl. In one variation, a heterocyclyl group having more than one ring where at least one ring is aryl, cycloalkyl, or heteroaryl is connected to the parent structure at an annular atom in the non-aromatic cyclic group having at least one heteroatom.

“Halo” or “halogen” refers to elements of the Group 17 series having atomic number 9 to 85. Preferred halo groups include the radicals of fluorine, chlorine, bromine and iodine. A haloalkyl is an alkyl group that is substituted with one or more halogens. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl.

“Carbonyl” refers to the group C═O.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

As used herein, the term “effective amount” intends such amount of a compound described herein which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “prodrug” as used herein refers to a compound which provides an active compound following administration to the individual in which it is used, by a chemical and/or biological process in vivo (e.g., by hydrolysis and/or an enzymatic conversion). The prodrug itself may be active, or it may be relatively inactive, then transformed into a more active compound. This disclosure embraces prodrugs of the compounds described herein.

When a moiety is indicated as substituted by “at least one” substituent, this also encompasses the disclosure of exactly one substituent.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

Terms and phrases used in this document, and variations thereof, unless otherwise expressly stated, should be construed as open ended as opposed to limiting. As examples of the foregoing: the term “including” should be read as meaning “including, without limitation” or the like; the term “for example” or “such as” is used to provide exemplary instances of the item in discussion, not an exhaustive or limiting list thereof. The section headings used herein are for organizational purposes only and are not to be construed as limiting the subject matter described.

### Compounds

In one aspect, provided is a compound of formula (I):

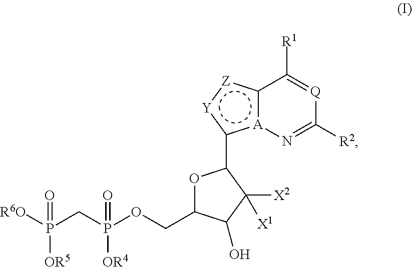

or a stereoisomer, tautomer, prodrug, or a pharmaceutically acceptable salt of any of the foregoing, wherein:

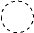

indicates a fully saturated, partially saturated, or aromatic ring;
X¹ and X² are each independently H, —CN, C₁₋₆ alkyl, —OR′, or halogen, wherein R′ is H, C₁₋₆ alkyl, C₃₋₁₂cycloalkyl, 3- to 12-membered heterocyclyl, 5- to 10-membered heteroaryl, or C₆₋₁₄ aryl;

### Q is N or CR³;

### Y is CH or N;

Z is CH, O, S, or N, provided that,

when Z is O, S, or N, then Y is CH,

when Z is CH, then Y is N, and

when Z is CH, O, or N, then Q is CR³;

### A is C or N;

R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl of R^{1a} and R^{1b} are each independently optionally substituted with R⁷, or

R^{1a} and R^{1b} are taken together with the nitrogen atom to which they attach to form a 3- to 12-membered heterocyclyl optionally substituted with C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, C₆₋₁₄ aryl, halogen, hydroxyl, C₁₋₆ alkoxy, or —CN, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently substituted with C₁₋₆ alkyl, halogen, hydroxyl, C₁₋₆ alkoxy, or —CN;

R² is H, C₁₋₆ alkyl, C₂₋₆ alkenyl, C₂₋₆ alkynyl, halogen, —CN, —NR^{2a}R^{2b}, —OR^{2a}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl of R² are each independently optionally substituted with R⁸, and wherein:

R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   R³ is H, C₁₋₆ alkyl, C₁₋₆ haloalkyl, halogen, or —CN;
   R⁴, R⁵, and R⁶ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
   each R⁷ is independently oxo, C₁₋₆ alkyl, C₂₋₆ alkenyl, C₂₋₆ alkynyl, halogen, —CN, —OR^{7a}, —SR^{7a}, —NR^{7a}R^{7b}, —NO₂, —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein the C₁₋₆ alkyl, C₃₋₆ cycloalkyl, 3- to 12-membered heterocyclyl, 5- to 10-membered heteroaryl, and C₆₋₁₄ aryl of R⁷ are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   each R⁸ is independently oxo, C₁₋₆ alkyl, C₂₋₆ alkenyl, C₂₋₆ alkynyl, halogen, —CN, —OR^{8a}, —SR^{8a}, —NR^{8a}R^{8b}, —NO₂, —C═NH(OR^{8a}), —C(O)R^{8a}, —OC(O)R^{8a}, —C(O)OR^{8a}, —C(O)NR^{8a}R^{8b}, —OC(O)NR^{8a}R^{8b}, —NR^{8a}C(O)R^{8b}, —NR^{8a}C(O)OR^{8b}, —S(O)R^{8a}, —S(O)₂R^{8a}, —NR^{8a}S(O)R^{8b}, —C(O)NR^{8a}S(O)R^{8b}, —NR^{8a}S(O)₂R^{8b}, —C(O)NR^{8a}S(O)₂R^{8b}, —S(O)NR^{8a}R^{8b}, —S(O)₂NR^{8a}R^{8b}, —P(O)(OR^{8a}) (OR^{8b}), C₃₋₆ cycloalkyl, 3- to 12-membered heterocyclyl, 5- to 10-membered heteroaryl, or C₆₋₁₄ aryl, wherein:

R^{8a} and R^{8b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{8a} and R^{8b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

In some embodiments of a compound of formula (I), or a stereoisomer, tautomer, prodrug, or a pharmaceutically acceptable salt of any of the foregoing,

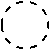

indicates an aromatic ring.

In some embodiments of a compound of formula (I), or a stereoisomer, tautomer, prodrug, or a pharmaceutically acceptable salt of any of the foregoing, Z is CH. In some embodiments, Z is O. In some embodiments, Z is N. In some embodiments, Z is S. In some embodiments, Z is CH, N, or S. In some embodiments, Z is CH, N, or O. In some embodiments, Z is CH or N.

In some embodiments of a compound of formula (I), or a stereoisomer, tautomer, prodrug, or a pharmaceutically acceptable salt of any of the foregoing, Y is CH. In some embodiments, Y is N. In some embodiments, Z is CH and Y is N. In some embodiments, Z is O and Y is CH. In some embodiments, Z is N and Y is CH. In some embodiments, Z is S and Y is CH.

In some embodiments of a compound of formula (I), or a stereoisomer, tautomer, prodrug, or a pharmaceutically acceptable salt of any of the foregoing, Q is N. In some embodiments, Q is CR³. In some embodiments, Z is S and Q is CR³. In some embodiments, Z is S and Q is N.

In some embodiments of a compound of formula (I), or a stereoisomer, tautomer, prodrug, or a pharmaceutically acceptable salt of any of the foregoing, A is N. In some embodiments, A is C. In some embodiments, Z is CH and A is N. In some embodiments, Z is CH and A is C. In some embodiments, Z is O and A is N. In some embodiments, Z is O and A is C. In some embodiments, Z is N and A is N. In some embodiments, Z is N and A is C. In some embodiments, Z is S and A is N. In some embodiments, Z is S and A is C. In some embodiments, Z is S; Q is CR³; and A is N. In some embodiments, Z is S; Q is CR³; and A is C. In some embodiments, Z is S; Q is N; and A is N. In some embodiments, Z is S; Q is N; and A is C.

In some embodiments, the compound of formula (I) is of formula (II), or a stereoisomer, tautomer, prodrug, or a pharmaceutically acceptable salt of any of the foregoing,

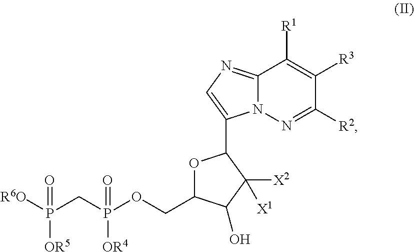

wherein X¹, X², and R¹-R⁶ are as defined herein for any embodiment of a compound of formula (I).

In some embodiments, the compound of formula (I) is of formula (III), or a stereoisomer, tautomer, prodrug, or a pharmaceutically acceptable salt of any of the foregoing,

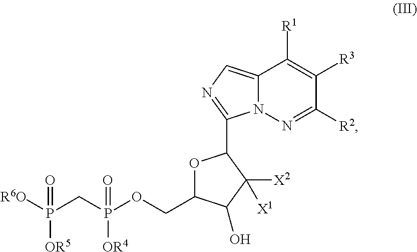

wherein X¹, X², and R¹-R⁶ are as defined herein for any embodiment of a compound of formula (I).

In some embodiments, the compound of formula (I) is of formula (IV), or a stereoisomer, tautomer, prodrug, or a pharmaceutically acceptable salt of any of the foregoing,

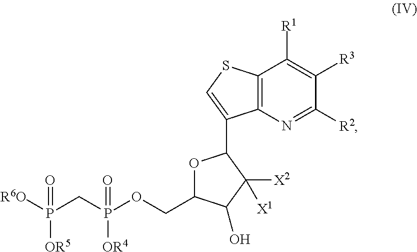

wherein X¹, X², and R¹-R⁶ are as defined herein for any embodiment of a compound of formula (I).

In some embodiments, the compound of formula (I) is of formula (V), or a stereoisomer, tautomer, prodrug, or a pharmaceutically acceptable salt of any of the foregoing,

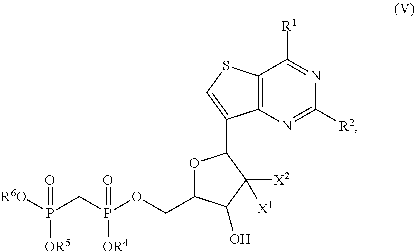

wherein X¹, X², R¹, R², and R⁴-R⁶ are as defined herein for any embodiment of a compound of formula (I).

In some embodiments, the compound of formula (I) is of any of the formulae provided below, or a stereoisomer, tautomer, prodrug, or a pharmaceutically acceptable salt of any of the foregoing.

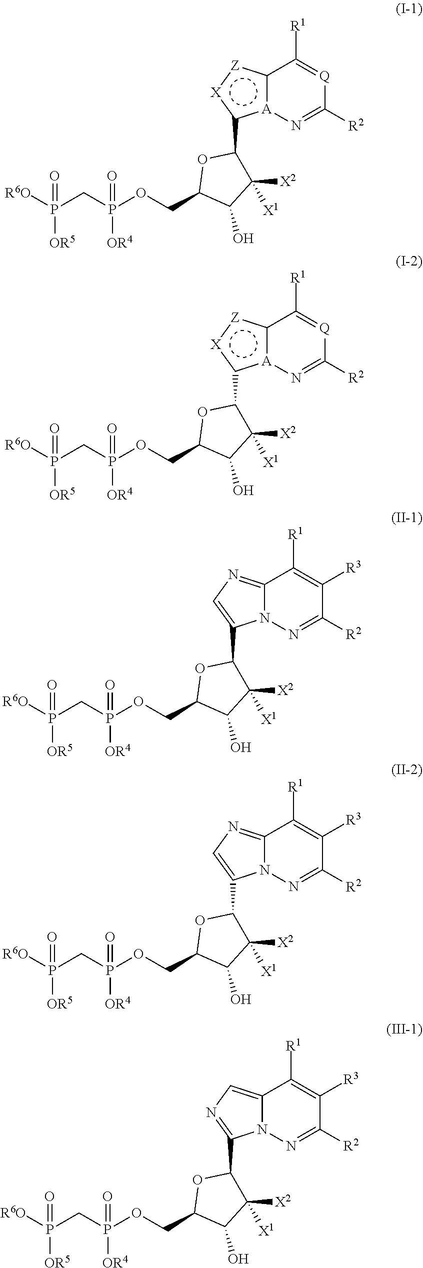

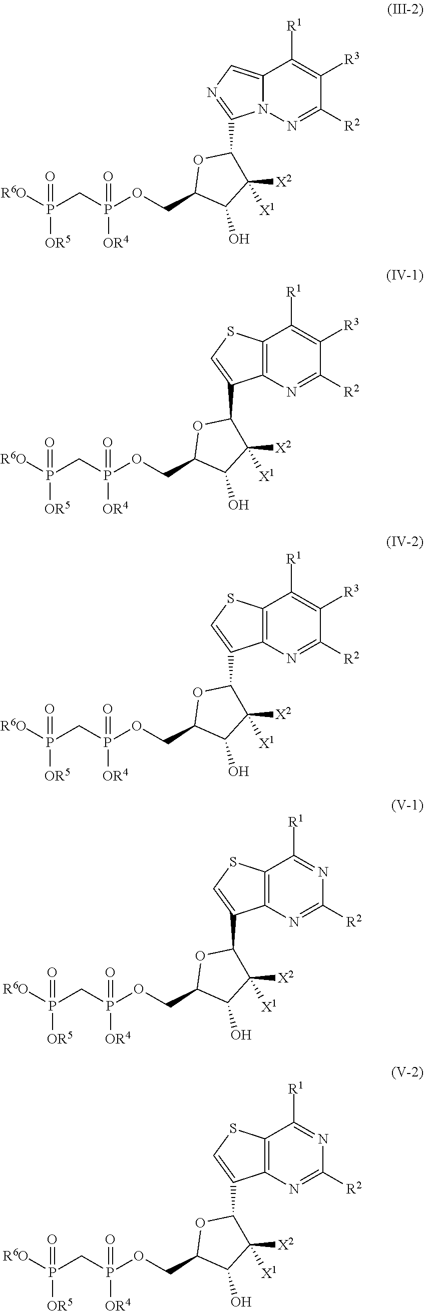

In some embodiments of a compound of formula (I) or any variation thereof where applicable (e.g., formulae II-IV, (I-1)-(IV-1), and (I-2)-(IV-2)), or a stereoisomer, tautomer, prodrug, or a pharmaceutically acceptable salt of any of the foregoing, the compound is not (((((2R,3S,4R,5S)-5-(6-chloro-8-(cyclopentyl(methyl)amino)imidazo[1,2-b]pyridazin-3-yl)-3,4-dihydroxytetrahydrofuran-2-yl)methoxy)(hydroxy)phosphoryl)methyl)phosphonic acid, or a stereoisomer, tautomer, prodrug, or a pharmaceutically acceptable salt of any of the foregoing. In some embodiments, the compound is not (((((2R,3S,4R,5S)-5-(6-chloro-8-(cyclopentyl(methyl)amino)imidazo[1,2-b]pyridazin-3-yl)-3,4-dihydroxytetrahydrofuran-2-yl)methoxy)(hydroxy)phosphoryl)methyl)phosphonic acid, or a stereoisomer, tautomer, or a pharmaceutically acceptable salt of any of the foregoing. In some embodiments, the compound is not (((((2R,3S,4R,5S)-5-(6-chloro-8-(cyclopentyl(methyl)amino)imidazo[1,2-b]pyridazin-3-yl)-3,4-dihydroxytetrahydrofuran-2-yl)methoxy)(hydroxy)phosphoryl)methyl)phosphonic acid or a pharmaceutically acceptable salt thereof.

In some embodiments of a compound of formula (I) or any variation thereof where applicable (e.g., formulae II-IV, (I-1)-(IV-1), and (I-2)-(IV-2)), or a stereoisomer, tautomer, prodrug, or a pharmaceutically acceptable salt of any of the foregoing, R³ is H. In some embodiments, R³ is C₁₋₆ alkyl or C₁₋₆ haloalkyl such as methyl, fluoromethyl, difluoromethyl, trifluoromethyl, ethyl, 2,2,2-trifluoroethyl, n-propyl, isopropyl, n-butyl, t-butyl, isobutyl, or sec-butyl. In some embodiments, R³ is halogen, such as fluoro, chloro, or bromo. In some embodiments, R³ is chloro. In some embodiments, R³ is fluoro. In some embodiments, R³ is —CN.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R⁴ is H. In some embodiments, R⁴ is C₁₋₆ alkyl, such as methyl, ethyl, n-propyl, isopropyl, n-butyl, t-butyl, isobutyl, or sec-butyl. In some embodiments, R⁴ is C₂₋₆ alkenyl, such as ethenyl, prop-1-enyl, prop-2-enyl, 2-methylprop-1-enyl, but-1-enyl, but-2-enyl, or but-3-enyl. In some embodiments, R⁴ is C₂₋₆ alkynyl, such as ethynyl, prop-1-ynyl, prop-2-ynyl, but-1-ynyl, but-2-ynyl, or but-3-ynyl. In some embodiments, R⁴ is C₃₋₁₂ cycloalkyl. In some embodiments, R⁴ is C₃₋₆ cycloalkyl, such as cyclopropyl, cyclobutyl, cyclopentyl, or cyclohexyl. In some embodiments, R⁴ is C₆₋₁₄ aryl, such as phenyl or naphthyl. In some embodiments, R⁴ is phenyl. In some embodiments, R⁴ is naphthyl. In some embodiments, R⁴ is 5- to 10-membered heteroaryl. In some embodiments, R⁴ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁴ is 3- to 12-membered heterocyclyl. In some embodiments, R⁴ is 5- or 6-membered heterocyclyl, such as tetrahydrofuranyl, pyrrolidinyl, piperidinyl, piperazinyl, morpholinyl, or thiomorpholinyl.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R⁵ is H. In some embodiments, R⁵ is C₁₋₆ alkyl, such as methyl, ethyl, n-propyl, isopropyl, n-butyl, t-butyl, isobutyl, or sec-butyl. In some embodiments, R⁵ is C₂₋₆ alkenyl, such as ethenyl, prop-1-enyl, prop-2-enyl, 2-methylprop-1-enyl, but-1-enyl, but-2-enyl, or but-3-enyl. In some embodiments, R⁵ is C₂₋₆ alkynyl, such as ethynyl, prop-1-ynyl, prop-2-ynyl, but-1-ynyl, but-2-ynyl, or but-3-ynyl. In some embodiments, R⁵ is C₃₋₁₂ cycloalkyl. In some embodiments, R⁵ is C₃₋₆ cycloalkyl, such as cyclopropyl, cyclobutyl, cyclopentyl, or cyclohexyl. In some embodiments, R⁵ is C₆₋₁₄ aryl, such as phenyl or naphthyl. In some embodiments, R⁵ is phenyl. In some embodiments, R⁵ is naphthyl. In some embodiments, R⁵ is 5- to 10-membered heteroaryl. In some embodiments, R⁵ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁵ is 3- to 12-membered heterocyclyl. In some embodiments, R⁵ is 5- or 6-membered heterocyclyl, such as tetrahydrofuranyl, pyrrolidinyl, piperidinyl, piperazinyl, morpholinyl, or thiomorpholinyl.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R⁶ is H. In some embodiments, R⁶ is C₁₋₆ alkyl, such as methyl, ethyl, n-propyl, isopropyl, n-butyl, t-butyl, isobutyl, or sec-butyl. In some embodiments, R⁶ is C₂₋₆ alkenyl, such as ethenyl, prop-1-enyl, prop-2-enyl, 2-methylprop-1-enyl, but-1-enyl, but-2-enyl, or but-3-enyl. In some embodiments, R⁶ is C₂₋₆ alkynyl, such as ethynyl, prop-1-ynyl, prop-2-ynyl, but-1-ynyl, but-2-ynyl, or but-3-ynyl. In some embodiments, R⁶ is C₃₋₁₂ cycloalkyl. In some embodiments, R⁶ is C₃₋₆ cycloalkyl, such as cyclopropyl, cyclobutyl, cyclopentyl, or cyclohexyl. In some embodiments, R⁶ is C₆₋₁₄ aryl, such as phenyl or naphthyl. In some embodiments, R⁶ is phenyl. In some embodiments, R⁶ is naphthyl. In some embodiments, R⁶ is 5- to 10-membered heteroaryl. In some embodiments, R⁶ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁶ is 3- to 12-membered heterocyclyl. In some embodiments, R⁶ is 5- or 6-membered heterocyclyl, such as tetrahydrofuranyl, pyrrolidinyl, piperidinyl, piperazinyl, morpholinyl, or thiomorpholinyl.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R³ is H; R⁴ is H; and R⁵ is H.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, X¹ is H. In some embodiments, X¹ is —CN. In some embodiments, X¹ is C₁₋₆ alkyl such as methyl, ethyl, n-propyl, isopropyl, n-butyl, t-butyl, isobutyl, or sec-butyl. In some embodiments, X¹ is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is —OH. In some embodiments, X¹ is halogen, such as fluoro, chloro, or bromo. In some embodiments, X¹ is fluoro. In some embodiments, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is H, halogen, or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is H, halogen, or —OH. In some embodiments, X¹ is H, fluoro, or —OH. In some embodiments, X¹ is H or halogen. In some embodiments, X¹ is H or fluoro. In some embodiments, X¹ is H or —OH.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, X² is H. In some embodiments, X² is —CN. In some embodiments, X² is C₁₋₆ alkyl such as methyl, ethyl, n-propyl, isopropyl, n-butyl, t-butyl, isobutyl, or sec-butyl. In some embodiments, X² is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is —OH. In some embodiments, X² is halogen, such as fluoro, chloro, or bromo. In some embodiments, X² is fluoro. In some embodiments, X² is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is H, halogen, or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is H, halogen, or —OH. In some embodiments, X² is H, fluoro, or —OH. In some embodiments, X² is H or halogen. In some embodiments, X² is H or fluoro. In some embodiments, X² is H or —OH.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, X¹ is H, halogen, or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl; and X² is H or halogen. In some embodiments, X¹ is H, halogen, or —OH; and X² is H or halogen. In some embodiments, X¹ is H, fluoro, or —OH; and X² is H or fluoro. In some embodiments, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl; and X² is H or halogen. In some embodiments, X¹ is H or —OH; and X² is H or halogen. In some embodiments, X¹ is H or —OH; and X² is H or fluoro.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R¹ is —NR^{1a}R^{1b}. In some embodiments, R¹ is —OR^{1a}.

In some embodiments of a compound of formula (I), or any related formula, or a stereoisomer, tautomer, prodrug, or a pharmaceutically acceptable salt of any of the foregoing, R^{1a} is H. In some embodiments, R^{1a} is C₁₋₆ alkyl optionally substituted with R⁷, such as methyl, ethyl, n-propyl, isopropyl, n-butyl, t-butyl, isobutyl, or sec-butyl, each of which is independently optionally substituted with R⁷. In some embodiments, R^{1a} is C₁₋₆ alkyl such as methyl, ethyl, n-propyl, isopropyl, n-butyl, t-butyl, isobutyl, or sec-butyl. In some embodiments, R^{1a} is methyl or ethyl, each of which is optionally substituted with R⁷. In some embodiments, R^{1a} is C₃₋₁₂ cycloalkyl optionally substituted with R⁷, such as cyclopropyl, cyclobutyl, cyclopentyl, or cyclohexyl, each of which is independently optionally substituted with R⁷. In some embodiments, R^{1a} is C₃₋₁₂ cycloalkyl which is unsubstituted, such as cyclopropyl, cyclobutyl, cyclopentyl, or cyclohexyl. In some embodiments, R^{1a} is C₃₋₁₂ cycloalkyl optionally substituted with R⁷, wherein the C₃₋₁₂ cycloalkyl is a monocyclic ring. In some embodiments, R^{1a} is C₃₋₁₂ cycloalkyl optionally substituted with R⁷, wherein the C₃₋₁₂ cycloalkyl is a fused or bridged ring. In some embodiments, R^{1a} is C₃₋₁₂ cycloalkyl optionally substituted with R⁷, wherein the C₃₋₁₂ cycloalkyl is a fused ring. In some embodiments, R^{1a} is C₃₋₁₂ cycloalkyl optionally substituted with R⁷, wherein the C₃₋₁₂ cycloalkyl is a bridged ring. In some embodiments, R^{1a} is

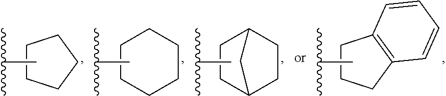

each of which is optionally substituted with R⁷. As used herein,

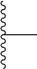

indicates the point of attachment to the remainder of the molecule. When

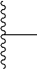

is not fixed at a specific annular atom of a ring, the point of attachment to the reminder of the molecule can be at any annular atom. In some embodiments, R^{1a} is C₆₋₁₄ aryl optionally substituted with R⁷, such as phenyl or naphthyl, each of which is independently optionally substituted with R⁷. In some embodiments, R^{1a} is C₆₋₁₄ aryl, which is unsubstituted, such as phenyl or naphthyl. In some embodiments, R^{1a} is phenyl optionally substituted with R⁷. In some embodiments, R^{1a} is phenyl. In some embodiments, R^{1a} is 5- to 10-membered heteroaryl optionally substituted with R⁷. In some embodiments, R^{1a} is 5- or 6-membered heteroaryl optionally substituted with R⁷, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁷. In some embodiments, R^{1a} is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R^{1a} is 3- to 12-membered heterocyclyl optionally substituted with R⁷. In some embodiments, R^{1a} is 5- or 6-membered heterocyclyl optionally substituted with R⁷, such as tetrahydrofuranyl, pyrrolidinyl, piperidinyl, piperazinyl, morpholinyl, or thiomorpholinyl, each of which is independently optionally substituted with R⁷. In some embodiments, R^{1a} is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R^{1a} is tetrahydrofuranyl optionally substituted with R⁷. In some embodiments, R^{1a} is C₁₋₆ alkyl, C₃₋₁₂ cycloalkyl, or 3- to 12-membered heterocyclyl, each of which is independently optionally substituted with R⁷. In some embodiments, Ria is C₃₋₁₂ cycloalkyl or 3- to 12-membered heterocyclyl, each of which is optionally substituted with R⁷. In some embodiments, R^{1a} is C₁₋₆ alkyl or 3- to 12-membered heterocyclyl, each of which is independently optionally substituted with R⁷.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R^{1a} is C₁₋₆ alkyl, C₃₋₁₂ cycloalkyl, or 3- to 12-membered heterocyclyl, each of which is independently optionally substituted with R⁷. In some embodiments, R^{1a} is

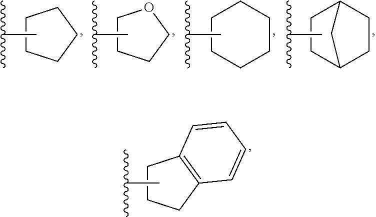

methyl, or ethyl, each of which is optionally substituted with R⁷. In some embodiments, R^{1a} is

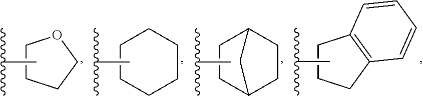

methyl, or ethyl, each of which is optionally substituted with R⁷.

In some embodiments of a compound of formula (I), or any related formula, or a stereoisomer, tautomer, prodrug, or a pharmaceutically acceptable salt of any of the foregoing, R^{1b} is H. In some embodiments, R^{1b} is C₁₋₆ alkyl optionally substituted with R⁷, such as methyl, ethyl, n-propyl, isopropyl, n-butyl, t-butyl, isobutyl, or sec-butyl, each of which is independently optionally substituted with R⁷. In some embodiments, R^{1b} is C₁₋₆ alkyl such as methyl, ethyl, n-propyl, isopropyl, n-butyl, t-butyl, isobutyl, or sec-butyl. In some embodiments, R^{1b} is methyl or ethyl, each of which is optionally substituted with R⁷. In some embodiments, R^{1b} is C₃₋₁₂ cycloalkyl optionally substituted with R⁷, such as cyclopropyl, cyclobutyl, cyclopentyl, or cyclohexyl, each of which is independently optionally substituted with R⁷. In some embodiments, R^{1b} is C₃₋₁₂ cycloalkyl which is unsubstituted, such as cyclopropyl, cyclobutyl, cyclopentyl, or cyclohexyl. In some embodiments, R^{1b} is C₃-12 cycloalkyl optionally substituted with R⁷, wherein the C₃₋₁₂ cycloalkyl is a monocyclic ring. In some embodiments, R^{1b} is C₃₋₁₂ cycloalkyl optionally substituted with R⁷, wherein the C₃₋₁₂ cycloalkyl is a fused or bridged ring. In some embodiments, R^{1b} is C₃₋₁₂ cycloalkyl optionally substituted with R⁷, wherein the C₃₋₁₂ cycloalkyl is a fused ring. In some embodiments, R^{1b} is C₃₋₁₂ cycloalkyl optionally substituted with R⁷, wherein the C₃₋₁₂ cycloalkyl is a bridged ring. In some embodiments, R^{1b} is C₃₋₁₂ cycloalkyl optionally substituted with R⁷, wherein the C₃₋₁₂ cycloalkyl is a bridged ring. In some embodiments, R^{1b} is

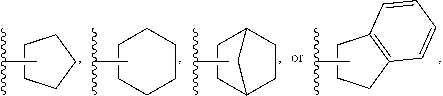

each of which is optionally substituted with R⁷.

In some embodiments, R^{1b} is C₆₋₁₄ aryl optionally substituted with R⁷, such as phenyl or naphthyl, each of which is independently optionally substituted with R⁷. In some embodiments, R^{1b} is C₆₋₁₄ aryl, which is unsubstituted, such as phenyl or naphthyl. In some embodiments, R^{1b} is phenyl optionally substituted with R⁷. In some embodiments, R^{1b} is phenyl. In some embodiments, R^{1b} is 5- to 10-membered heteroaryl optionally substituted with R⁷. In some embodiments, R^{1b} is 5- or 6-membered heteroaryl optionally substituted with R⁷, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁷. In some embodiments, R^{1b} is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R^{1b} is 3- to 12-membered heterocyclyl optionally substituted with R⁷. In some embodiments, R^{1b} is 5- or 6-membered heterocyclyl optionally substituted with R⁷, such as tetrahydrofuranyl, pyrrolidinyl, piperidinyl, piperazinyl, morpholinyl, or thiomorpholinyl, each of which is independently optionally substituted with R⁷. In some embodiments, R^{1b} is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R^{1b} is tetrahydrofuranyl optionally substituted with R⁷.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R^{1b} is C₁₋₆ alkyl, C₃₋₁₂ cycloalkyl, or 3- to 12-membered heterocyclyl, each of which is independently optionally substituted with R⁷. In some embodiments, R^{1b} is

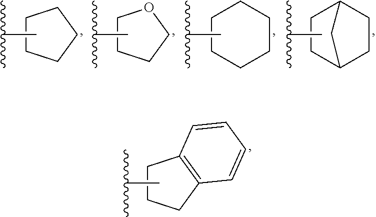

methyl, or ethyl, each of which is optionally substituted with R⁷. In some embodiments, R^{1b} is

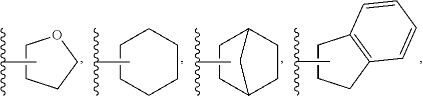

methyl, or ethyl, each of which is optionally substituted with R⁷. In some embodiments, R^{1b} is H or C₁₋₆ alkyl. In some embodiments, R^{1b} is H or methyl.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R¹ is —NR^{1a}R^{1b}; R^{1a} is C₁₋₆ alkyl, C₃₋₁₂ cycloalkyl, or 3- to 12-membered heterocyclyl, each of which is independently optionally substituted with R⁷; and R^{1b} is H or C₁₋₆ alkyl. In some embodiments, R¹ is —NR^{1a}R^{1b}; R^{1a} is C₁₋₆ alkyl or 3- to 12-membered heterocyclyl, each of which is independently optionally substituted with R⁷; and R^{1b} is H or C₁₋₆ alkyl. In some embodiments, R¹ is —NR^{1a}R^{1b}; R^{1a} is

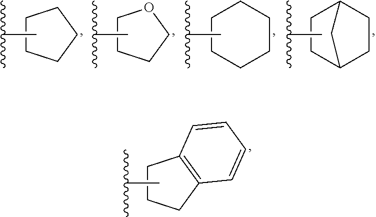

methyl, or ethyl, each of which is optionally substituted with R⁷; and R^{1b} is H or methyl. In some embodiments, R¹ is —OR^{1a}; and R^{1a} is C₃₋₁₂ cycloalkyl or 3- to 12-membered heterocyclyl, each of which is optionally substituted with R⁷. In some embodiments, R¹ is —OR^{1a}; and R^{1a} is C₃₋₁₂ cycloalkyl optionally substituted with R⁷.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R⁷ is halogen or phenyl optionally substituted with halogen. In some embodiments, R⁷ is halogen such as fluoro, chloro, or bromo. In some embodiments, R⁷ is fluoro. In some embodiments, R⁷ is phenyl optionally substituted with halogen. In some embodiments, R⁷ is phenyl optionally substituted with fluoro or chloro. In some embodiments, R⁷ is

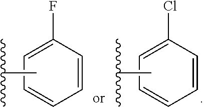

In some embodiments, R⁷ is fluoro,

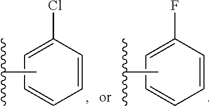

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R¹ is —NR^{1a}R^{1b}; and R^{1a} is C₁₋₆ alkyl, C₃₋₁₂ cycloalkyl, or 3- to 12-membered heterocyclyl, each of which is independently optionally substituted with R⁷; R^{1b} is H or C₁₋₆ alkyl; and R⁷ is halogen or phenyl optionally substituted with halogen. In some embodiments, R¹ is —NR^{1a}R^{1b}; and R^{1a} is C₁₋₆ alkyl or 3- to 12-membered heterocyclyl, each of which is independently optionally substituted with R⁷; R^{1b} is H or C₁₋₆ alkyl; and R⁷ is halogen or phenyl optionally substituted with halogen. In some embodiments, when Z is N, A is N, and R¹ is —NR^{1a}R^{1b}, then R^{1a} is C₁₋₆ alkyl, C₃₋₁₂cycloalkyl, or 3- to 12-membered heterocyclyl, each of which is independently optionally substituted with R⁷ and R^{1b} is H. In some embodiments, R¹ is —NR^{1a}R^{1b}; R^{1a} is C₁₋₆ alkyl, C₃₋₁₂cycloalkyl, or 3- to 12-membered heterocyclyl, each of which is independently optionally substituted with R⁷; and R^{1b} is H.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R^{1a} and R^{1b} are each independently H, methyl,

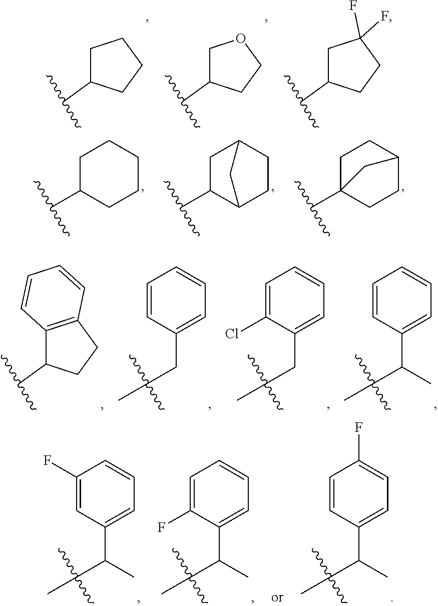

In some embodiments, R¹ is —NR^{1a}R^{1b}; R^{1a} is

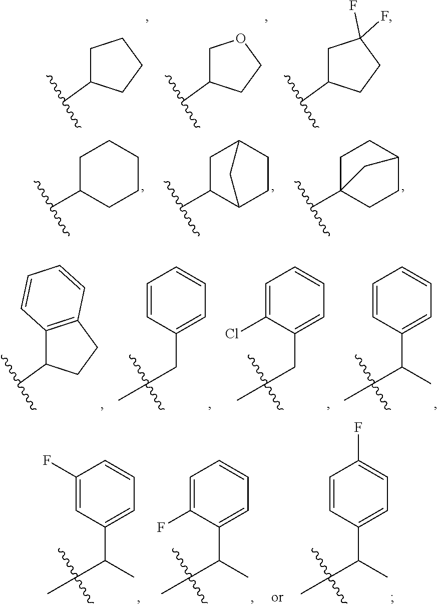

and R^{1b} is H or methyl. In some embodiments, when Z is N, A is N, and R¹ is —NR^{1a}R^{1b}, then R^{1a} is

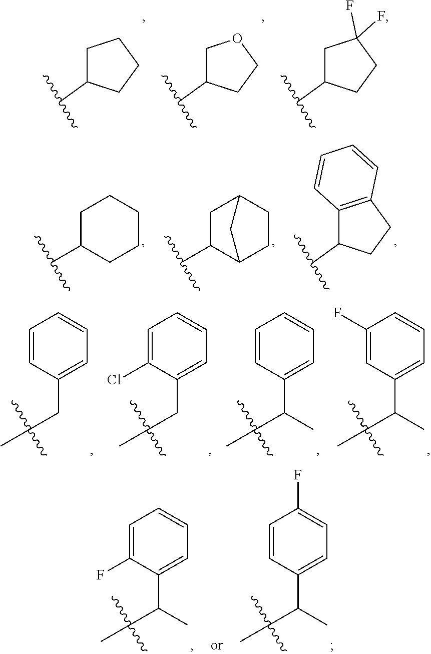

### and R^{1b} is H.

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R^{1a} and R^{1b} are taken together with the nitrogen atom to which they attach to form a 3- to 12-membered heterocyclyl optionally substituted with C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, C₆₋₁₄ aryl, halogen hydroxyl, C₁₋₆ alkoxy, or —CN, wherein the C₁₋₆alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently substituted with C₁₋₆ alkyl, halogen, hydroxyl, C₁₋₆ alkoxy, or —CN. In some embodiments, R^{1a} and R^{1b} are taken together with the nitrogen atom to which they attach to form a 3- to 12-membered heterocyclyl optionally substituted with halogen or phenyl optionally substituted with halogen. In some embodiments, R^{1a} and R^{1b} are taken together with the nitrogen atom to which they attach to form a 3- to 12-membered heterocyclyl which is unsubstituted. In some embodiments, R^{1a} and R^{1b} are taken together with the nitrogen atom to which they attach to form a moiety selected from the group consisting of:

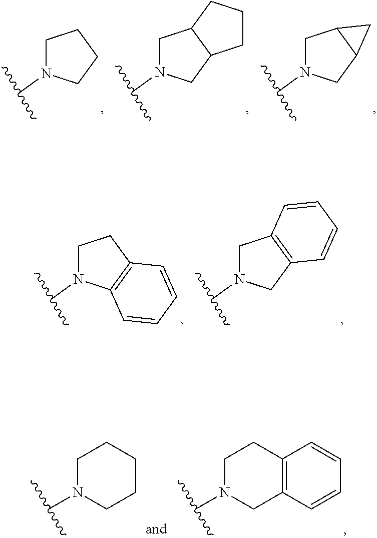

each of which is optionally substituted with halogen or phenyl optionally substituted with halogen. In some embodiments, R^{1a} and R^{1b} are taken together with the nitrogen atom to which they attach to form a moiety selected from the group consisting of:

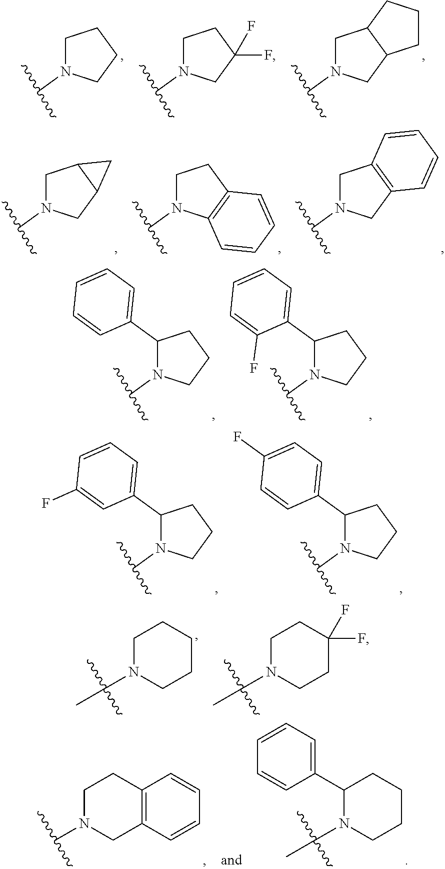

In some embodiments of a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, R¹ is selected from the group consisting of:

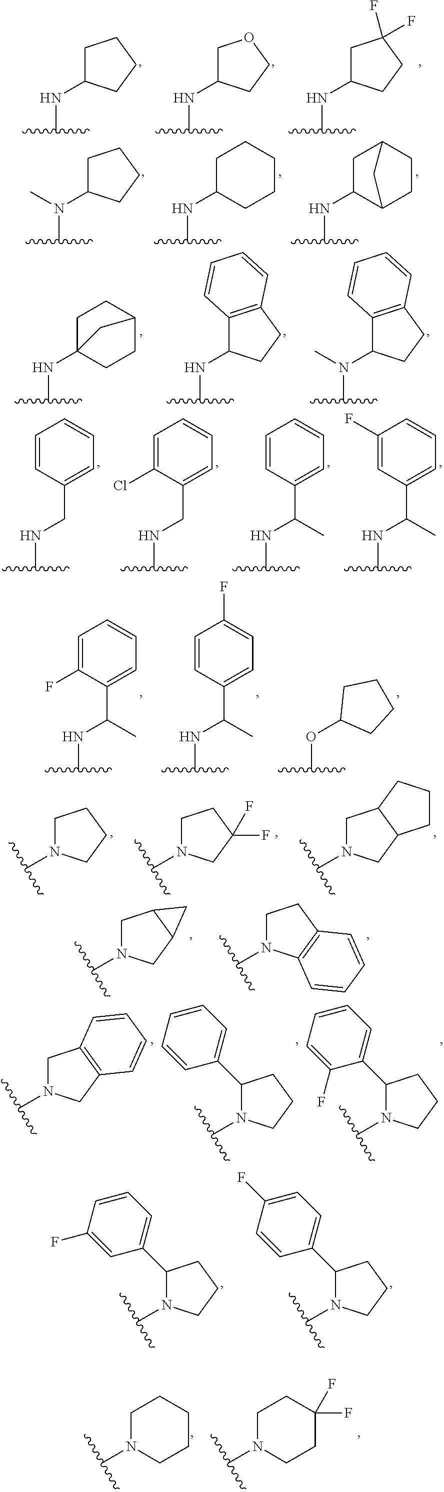

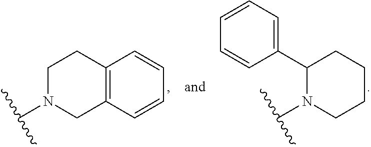 [SMILES: *N1CCc2ccccc2C1 ; *N1CCCCC1c1ccccc1]

In some embodiments of a compound of formula (I), or any related formula, or a stereoisomer, tautomer, prodrug, or a pharmaceutically acceptable salt of any of the foregoing, R² is H, C₁₋₆ alkyl, C₂₋₆ alkenyl, C₂₋₆ alkynyl, halogen, —CN, —OR^{2a}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl of R² are each independently optionally substituted with R⁸. In some embodiments, R² is C₁₋₆ alkyl optionally substituted with R⁸, such as methyl, ethyl, n-propyl, isopropyl, n-butyl, t-butyl, isobutyl, or sec-butyl, each of which is independently optionally substituted with R⁸. In some embodiments, R² is C₁₋₆ alkyl such as methyl, ethyl, n-propyl, isopropyl, n-butyl, t-butyl, isobutyl, or sec-butyl. In some embodiments, R² is C₂₋₆ alkenyl optionally substituted with R⁸, such as ethenyl, prop-1-enyl, prop-2-enyl, 2-methylprop-1-enyl, but-1-enyl, but-2-enyl, or but-3-enyl, each of which is independently optionally substituted with R⁸. In some embodiments, R² is C₂₋₆ alkenyl, such as ethenyl, prop-1-enyl, prop-2-enyl, 2-methylprop-1-enyl, but-1-enyl, but-2-enyl, or but-3-enyl. In some embodiments, R² is C₂₋₆ alkynyl optionally substituted with R⁸, such as ethynyl, prop-1-ynyl, prop-2-ynyl, but-1-ynyl, but-2-ynyl, or but-3-ynyl, each of which is independently optionally substituted with R⁸. In some embodiments, R² is C₂₋₆ alkynyl, such as ethynyl, prop-1-ynyl, prop-2-ynyl, but-1-ynyl, but-2-ynyl, or but-3-ynyl. In some embodiments, R² is C₃₋₁₂ cycloalkyl optionally substituted with R⁸. In some embodiments, R² is C₃₋₆ cycloalkyl optionally substituted with R⁸, such as cyclopropyl, cyclobutyl, cyclopentyl, or cyclohexyl, each of which is independently optionally substituted with R⁸. In some embodiments, R² is C₃₋₆ cycloalkyl which is unsubstituted, such as cyclopropyl, cyclobutyl, cyclopentyl, or cyclohexyl. In some embodiments, R² is C₆₋₁₄ aryl optionally substituted with R⁸, such as phenyl or naphthyl, each of which is independently optionally substituted with R⁸. In some embodiments, R² is C₆₋₁₄ aryl, which is unsubstituted, such as phenyl or naphthyl. In some embodiments, R² is phenyl optionally substituted with R⁸. In some embodiments, R² is phenyl. In some embodiments, R² is 5- to 10-membered heteroaryl optionally substituted with R⁸. In some embodiments, R² is 5- or 6-membered heteroaryl optionally substituted with R⁸, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁸. In some embodiments, R² is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R² is 3- to 12-membered heterocyclyl optionally substituted with R⁸. In some embodiments, R² is 5- or 6-membered heterocyclyl optionally substituted with R⁸, such as tetrahydrofuranyl, pyrrolidinyl, piperidinyl, piperazinyl, morpholinyl, or thiomorpholinyl, each of which is independently optionally substituted with R⁸. In some embodiments, R² is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R² is H, —CN, or halogen. In some embodiments, R² is H. In some embodiments, R² is —CN. In some embodiments, R² is halogen, such as fluoro, chloro, or bromo. In some embodiments, R² is chloro.

It is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (I) may be combined with every description, variation, embodiment or aspect of Y, Z, A, Q,

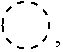

X¹, X², and R²-R⁵ the same as if each and every combination were specifically and individually listed. It is also understood that all descriptions, variations, embodiments or aspects of formula (I), where applicable, apply equally to other formulae detailed herein (e.g., formulae II-V, (I-1)-(V-1), and (I-2)-(V-2)), and are equally described, the same as if each and every description, variation, embodiment or aspect were separately and individually listed for all formulae. For example, in some embodiments, provided is a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, wherein X¹ is H or —OH; X² is H or halogen; R¹ is —NR^{1a}R^{1b} or —OR^{1a}; R^{1a} is C₁₋₆ alkyl, C₃₋₁₂cycloalkyl, or 3- to 12-membered heterocyclyl, each of which is independently optionally substituted with R⁷, wherein R⁷ is halogen or phenyl optionally substituted with halogen; R^{1b} is H or C₁₋₆ alkyl, or R^{1a} and R^{1b} are taken together with the nitrogen atom to which they attach to form a 3- to 12-membered heterocyclyl optionally substituted with halogen or phenyl optionally substituted with halogen; R² is H, —CN, or halogen; R⁴ is H; R⁵ is H; and R⁶ is H. As another example, in some embodiments, provided is a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, wherein X¹ is H or —OH; X² is H or halogen; R¹ is —NR^{1a}R^{1b} or —OR^{1a}; R^{1a} is C₁₋₆ alkyl, C₃₋₁₂cycloalkyl, or 3- to 12-membered heterocyclyl, each of which is independently optionally substituted with R⁷, wherein R⁷ is halogen or phenyl optionally substituted with halogen; R^{1b} is H or C₁₋₆ alkyl, or R^{1a} and R^{1b} are taken together with the nitrogen atom to which they attach to form a 3- to 12-membered heterocyclyl optionally substituted with halogen or phenyl optionally substituted with halogen; R² is halogen; R⁴ is H; R⁵ is H; and R⁶ is H. As another example, in some embodiments, provided is a compound of formula (I) or any variation thereof where applicable (e.g., formulae II-V, (I-1)-(V-1), and (I-2)-(V-2)), or a stereoisomer, tautomer, prodrug, or a pharmaceutically acceptable salt of any of the foregoing, wherein X¹ is H or —OH; X² is H or halogen; R¹ is —NR^{1a}R^{1b} or —OR^{1a}; R^{1a} is C₁₋₆ alkyl or 3- to 12-membered heterocyclyl, each of which is independently optionally substituted with R⁷, wherein R⁷ is halogen or phenyl optionally substituted with halogen; R^{1b} is H or C₁₋₆ alkyl, or R^{1a} and R^{1b} are taken together with the nitrogen atom to which they attach to form a 3- to 12-membered heterocyclyl optionally substituted with halogen or phenyl optionally substituted with halogen; R² is halogen; R⁴ is H; R⁵ is H; and R⁶ is H.

In some embodiments, provided is a compound selected from the compounds in Table 1, or a stereoisomer, tautomer, solvate, prodrug or salt thereof. In some embodiments, provided is a compound selected from the compounds in Table 1, or a stereoisomer, tautomer, prodrug, or a pharmaceutically acceptable salt of any of the foregoing. In some embodiments, provided is a compound selected from the compounds in Table 1, or a pharmaceutically acceptable salt thereof. In some embodiments, provided is a compound selected from the compounds in Table 1. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| No. | Structure |
|---|---|
| 1 | 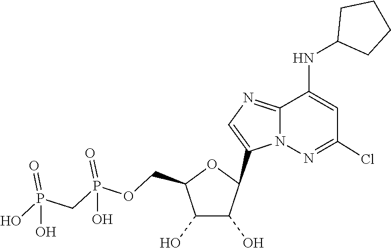 [SMILES: O=P(O)(O)CP(=O)(O)OC[C@H]1O[C@@H](c2cnc3c(NC4CCCC4)cc(Cl)nn23)[C@H](O)[C@@H]1O] |
| 2 | 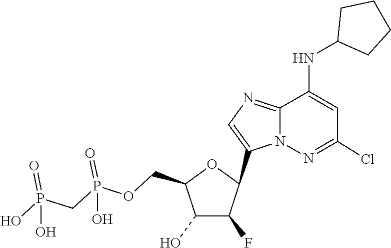 [SMILES: O=P(O)(O)CP(=O)(O)OC[C@H]1O[C@@H](c2cnc3c(NC4CCCC4)cc(Cl)nn23)[C@@H](F)[C@@H]1O] |
| 3 | 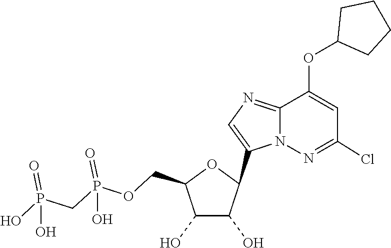 [SMILES: O=P(O)(O)CP(=O)(O)OC[C@H]1O[C@@H](c2cnc3c(OC4CCCC4)cc(Cl)nn23)[C@H](O)[C@@H]1O] |
| 4 | 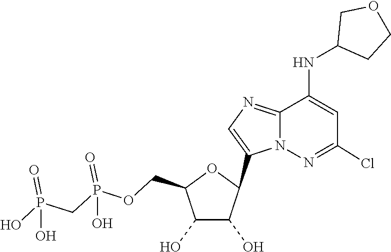 [SMILES: O=P(O)(O)CP(=O)(O)OC[C@H]1O[C@@H](c2cnc3c(NC4CCOC4)cc(Cl)nn23)[C@H](O)[C@@H]1O] |
| 5 | 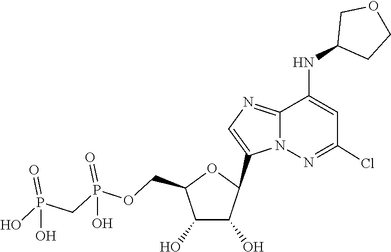 [SMILES: O=P(O)(O)CP(=O)(O)OC[C@H]1O[C@@H](c2cnc3c(N[C@@H]4CCOC4)cc(Cl)nn23)[C@H](O)[C@@H]1O] |
| 6 | 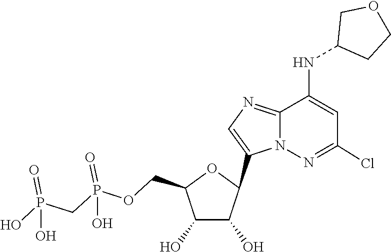 |
| 7 | 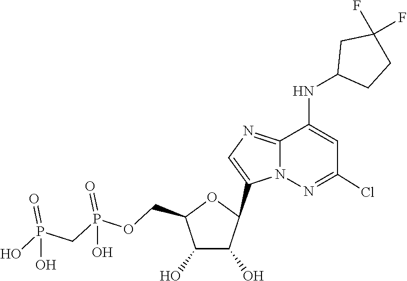 [SMILES: O=P(O)(O)CP(=O)(O)OC[C@H]1O[C@@H](c2cnc3c(NC4CCC(F)(F)C4)cc(Cl)nn23)[C@H](O)[C@@H]1O] |
| 8 | 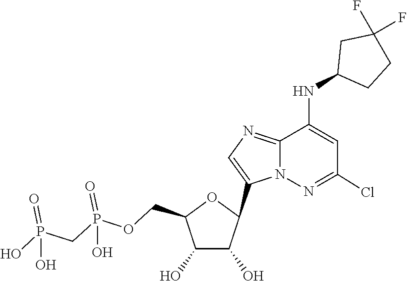 [SMILES: O=P(O)(O)CP(=O)(O)OC[C@H]1O[C@@H](c2cnc3c(N[C@@H]4CCC(F)(F)C4)cc(Cl)nn23)[C@H](O)[C@@H]1O] |
| 9 | 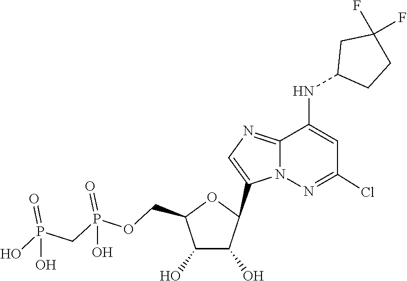 |
| 10 | 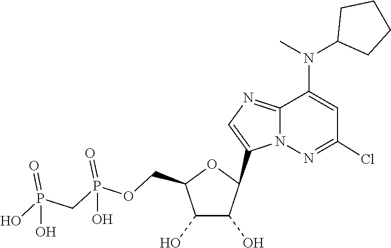 [SMILES: CN(c1cc(Cl)nn2c([C@@H]3O[C@H](COP(=O)(O)CP(=O)(O)O)[C@@H](O)[C@H]3O)cnc12)C1CCCC1] |
| 11 | 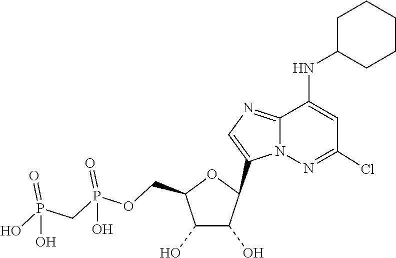 [SMILES: O=P(O)(O)CP(=O)(O)OC[C@H]1O[C@@H](c2cnc3c(NC4CCCCC4)cc(Cl)nn23)[C@H](O)[C@@H]1O] |
| 12 | 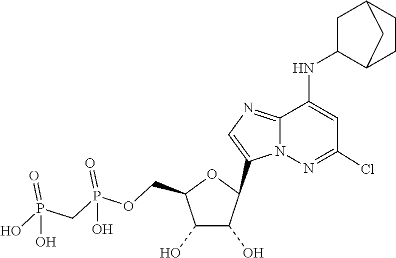 [SMILES: O=P(O)(O)CP(=O)(O)OC[C@H]1O[C@@H](c2cnc3c(NC4CC5CCC4C5)cc(Cl)nn23)[C@H](O)[C@@H]1O] |
| 13 | 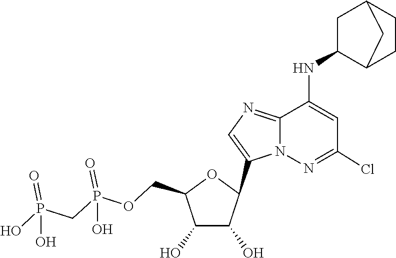 [SMILES: O=P(O)(O)CP(=O)(O)OC[C@H]1O[C@@H](c2cnc3c(N[C@H]4CC5CCC4C5)cc(Cl)nn23)[C@H](O)[C@@H]1O] |
| 14 | 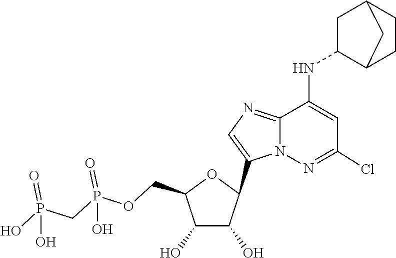 |
| 15 | 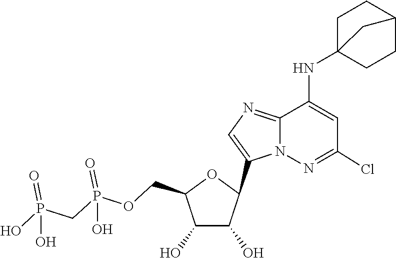 [SMILES: O=P(O)(O)CP(=O)(O)OC[C@H]1O[C@@H](c2cnc3c(NC45CCC(CC4)C5)cc(Cl)nn23)[C@H](O)[C@@H]1O] |
| 16 | 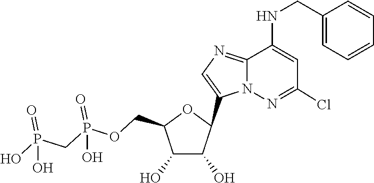 [SMILES: O=P(O)(O)CP(=O)(O)OC[C@H]1O[C@@H](c2cnc3c(NCc4ccccc4)cc(Cl)nn23)[C@H](O)[C@@H]1O] |
| 17 | 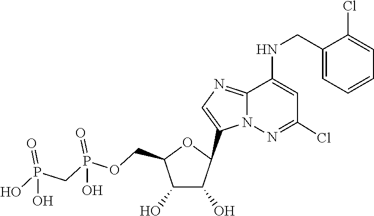 [SMILES: O=P(O)(O)CP(=O)(O)OC[C@H]1O[C@@H](c2cnc3c(NCc4ccccc4Cl)cc(Cl)nn23)[C@H](O)[C@@H]1O] |
| 18 | 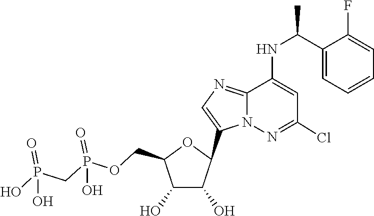 [SMILES: C[C@H](Nc1cc(Cl)nn2c([C@@H]3O[C@H](COP(=O)(O)CP(=O)(O)O)[C@@H](O)[C@H]3O)cnc12)c1ccccc1F] |
| 19 | 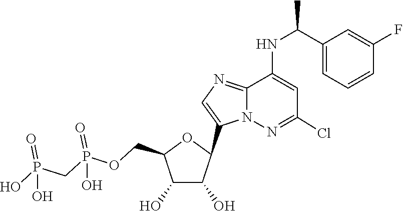 [SMILES: C[C@H](Nc1cc(Cl)nn2c([C@@H]3O[C@H](COP(=O)(O)CP(=O)(O)O)[C@@H](O)[C@H]3O)cnc12)c1cccc(F)c1] |
| 20 | 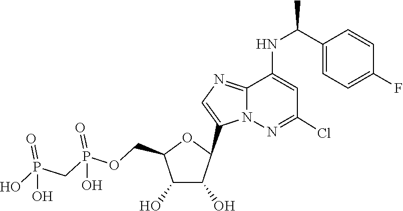 [SMILES: C[C@H](Nc1cc(Cl)nn2c([C@@H]3O[C@H](COP(=O)(O)CP(=O)(O)O)[C@@H](O)[C@H]3O)cnc12)c1ccc(F)cc1] |
| 21 | 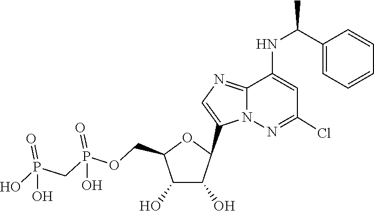 [SMILES: C[C@H](Nc1cc(Cl)nn2c([C@@H]3O[C@H](COP(=O)(O)CP(=O)(O)O)[C@@H](O)[C@H]3O)cnc12)c1ccccc1] |
| 22 | 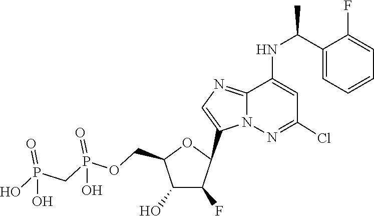 [SMILES: C[C@H](Nc1cc(Cl)nn2c([C@@H]3O[C@H](COP(=O)(O)CP(=O)(O)O)[C@@H](O)[C@@H]3F)cnc12)c1ccccc1F] |
| 23 | 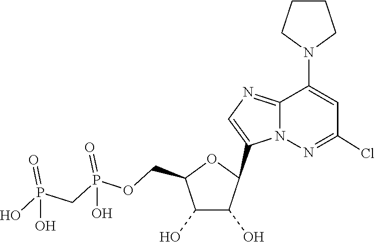 [SMILES: O=P(O)(O)CP(=O)(O)OC[C@H]1O[C@@H](c2cnc3c(N4CCCC4)cc(Cl)nn23)[C@H](O)[C@@H]1O] |
| 24 | 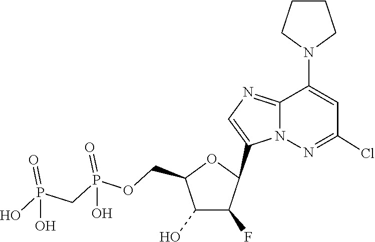 [SMILES: O=P(O)(O)CP(=O)(O)OC[C@H]1O[C@@H](c2cnc3c(N4CCCC4)cc(Cl)nn23)[C@@H](F)[C@@H]1O] |
| 25 | 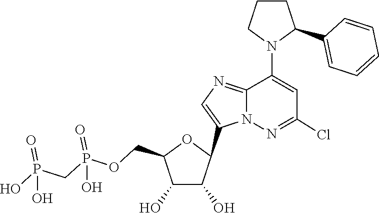 [SMILES: O=P(O)(O)CP(=O)(O)OC[C@H]1O[C@@H](c2cnc3c(N4CCC[C@H]4c4ccccc4)cc(Cl)nn23)[C@H](O)[C@@H]1O] |
| 26 | 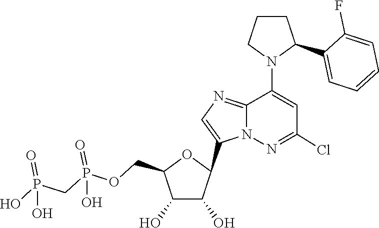 [SMILES: O=P(O)(O)CP(=O)(O)OC[C@H]1O[C@@H](c2cnc3c(N4CCC[C@H]4c4ccccc4F)cc(Cl)nn23)[C@H](O)[C@@H]1O] |
| 27 | 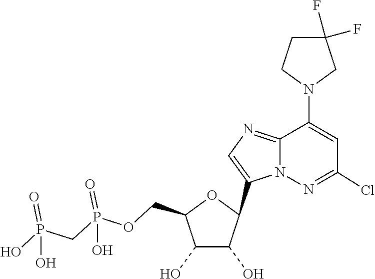 [SMILES: O=P(O)(O)CP(=O)(O)OC[C@H]1O[C@@H](c2cnc3c(N4CCC(F)(F)C4)cc(Cl)nn23)[C@H](O)[C@@H]1O] |
| 28 | 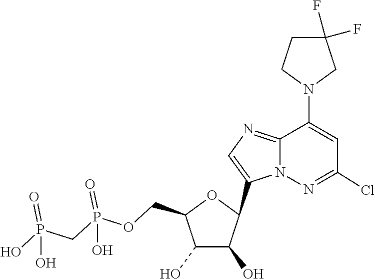 [SMILES: O=P(O)(O)CP(=O)(O)OC[C@H]1O[C@@H](c2cnc3c(N4CCC(F)(F)C4)cc(Cl)nn23)[C@@H](O)[C@@H]1O] |
| 29 | 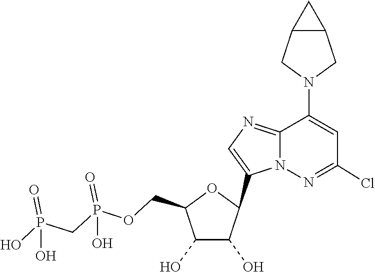 [SMILES: O=P(O)(O)CP(=O)(O)OC[C@H]1O[C@@H](c2cnc3c(N4CC5CC5C4)cc(Cl)nn23)[C@H](O)[C@@H]1O] |
| 30 | 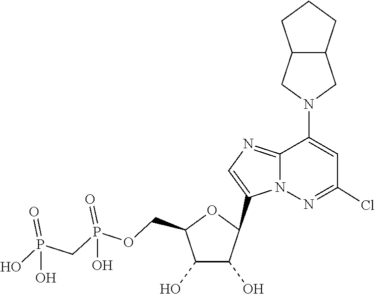 [SMILES: O=P(O)(O)CP(=O)(O)OC[C@H]1O[C@@H](c2cnc3c(N4CC5CCCC5C4)cc(Cl)nn23)[C@H](O)[C@@H]1O] |
| 31 | 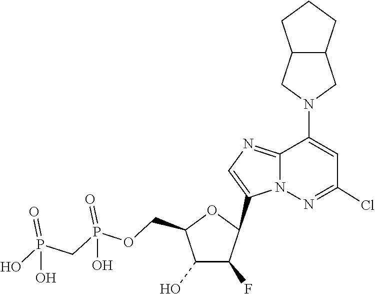 [SMILES: O=P(O)(O)CP(=O)(O)OC[C@H]1O[C@@H](c2cnc3c(N4CC5CCCC5C4)cc(Cl)nn23)[C@@H](F)[C@@H]1O] |
| 32 | 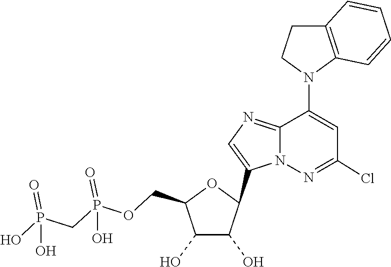 [SMILES: O=P(O)(O)CP(=O)(O)OC[C@H]1O[C@@H](c2cnc3c(N4CCc5ccccc54)cc(Cl)nn23)[C@H](O)[C@@H]1O] |
| 33 | 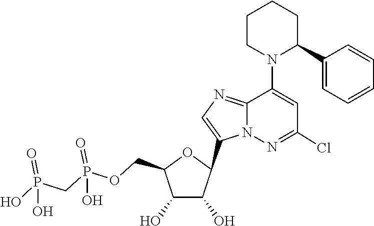 [SMILES: O=P(O)(O)CP(=O)(O)OC[C@H]1O[C@@H](c2cnc3c(N4CCCC[C@H]4c4ccccc4)cc(Cl)nn23)[C@H](O)[C@@H]1O] |
| 34 | 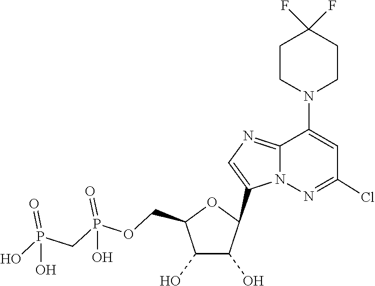 [SMILES: O=P(O)(O)CP(=O)(O)OC[C@H]1O[C@@H](c2cnc3c(N4CCC(F)(F)CC4)cc(Cl)nn23)[C@H](O)[C@@H]1O] |
| 35 | 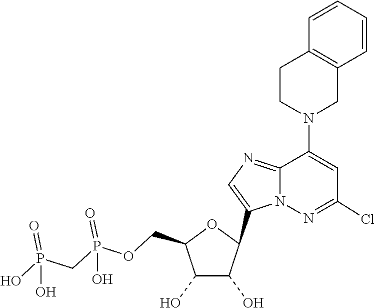 [SMILES: O=P(O)(O)CP(=O)(O)OC[C@H]1O[C@@H](c2cnc3c(N4CCc5ccccc5C4)cc(Cl)nn23)[C@H](O)[C@@H]1O] |
| 36 | 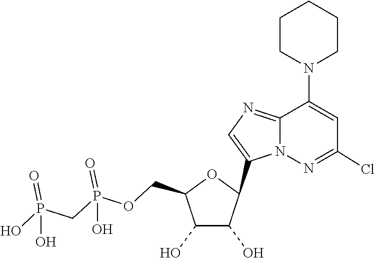 [SMILES: O=P(O)(O)CP(=O)(O)OC[C@H]1O[C@@H](c2cnc3c(N4CCCCC4)cc(Cl)nn23)[C@H](O)[C@@H]1O] |
| 37 | 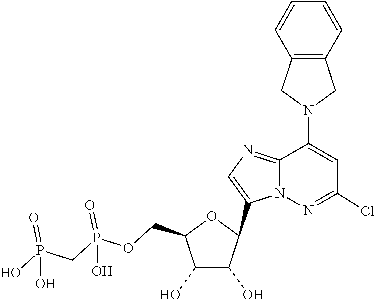 [SMILES: O=P(O)(O)CP(=O)(O)OC[C@H]1O[C@@H](c2cnc3c(N4Cc5ccccc5C4)cc(Cl)nn23)[C@H](O)[C@@H]1O] |
| 38 | 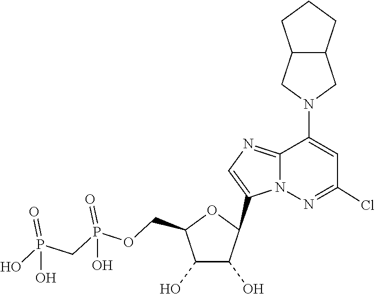 [SMILES: O=P(O)(O)CP(=O)(O)OC[C@H]1O[C@@H](c2cnc3c(N4CC5CCCC5C4)cc(Cl)nn23)[C@H](O)[C@@H]1O] |
| 39 | 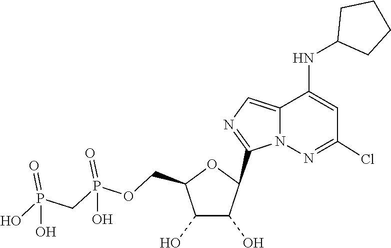 [SMILES: O=P(O)(O)CP(=O)(O)OC[C@H]1O[C@@H](c2ncc3c(NC4CCCC4)cc(Cl)nn23)[C@H](O)[C@@H]1O] |
| 40 | 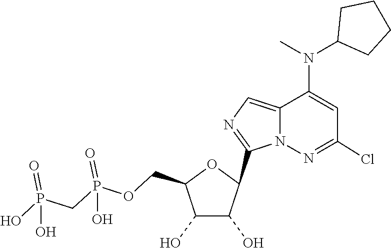 [SMILES: CN(c1cc(Cl)nn2c([C@@H]3O[C@H](COP(=O)(O)CP(=O)(O)O)[C@@H](O)[C@H]3O)ncc12)C1CCCC1] |
| 41 | 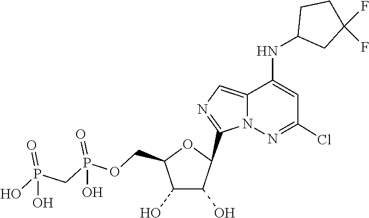 [SMILES: O=P(O)(O)CP(=O)(O)OC[C@H]1O[C@@H](c2ncc3c(NC4CCC(F)(F)C4)cc(Cl)nn23)[C@H](O)[C@@H]1O] |
| 42 | 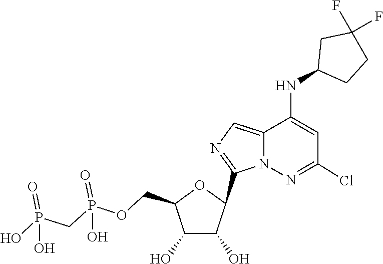 [SMILES: O=P(O)(O)CP(=O)(O)OC[C@H]1O[C@@H](c2ncc3c(N[C@@H]4CCC(F)(F)C4)cc(Cl)nn23)[C@H](O)[C@@H]1O] |
| 43 | 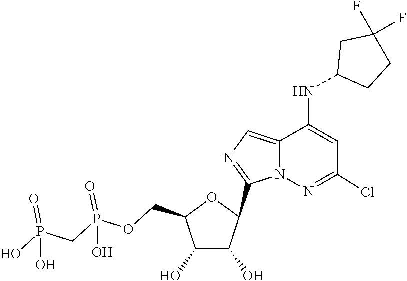 |
| 44 | 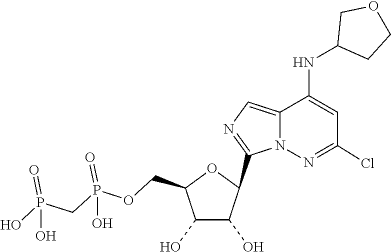 [SMILES: O=P(O)(O)CP(=O)(O)OC[C@H]1O[C@@H](c2ncc3c(NC4CCOC4)cc(Cl)nn23)[C@H](O)[C@@H]1O] |
| 45 | 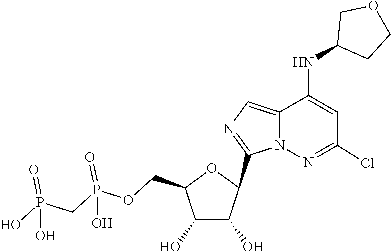 [SMILES: O=P(O)(O)CP(=O)(O)OC[C@H]1O[C@@H](c2ncc3c(N[C@@H]4CCOC4)cc(Cl)nn23)[C@H](O)[C@@H]1O] |
| 46 | 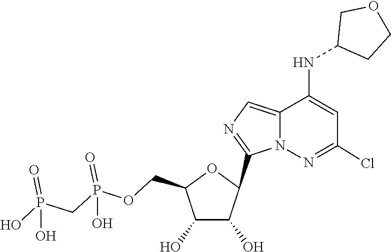 |
| 47 | 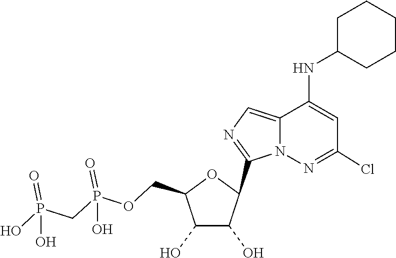 [SMILES: O=P(O)(O)CP(=O)(O)OC[C@H]1O[C@@H](c2ncc3c(NC4CCCCC4)cc(Cl)nn23)[C@H](O)[C@@H]1O] |
| 48 | 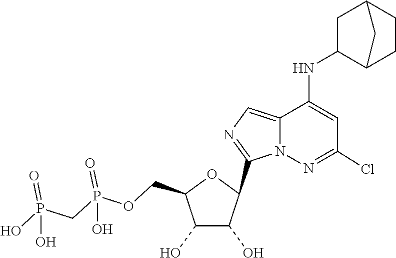 [SMILES: O=P(O)(O)CP(=O)(O)OC[C@H]1O[C@@H](c2ncc3c(NC4CC5CCC4C5)cc(Cl)nn23)[C@H](O)[C@@H]1O] |
| 49 | 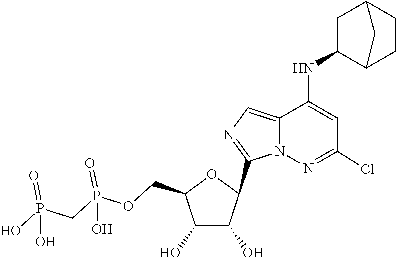 [SMILES: O=P(O)(O)CP(=O)(O)OC[C@H]1O[C@@H](c2ncc3c(N[C@H]4CC5CCC4C5)cc(Cl)nn23)[C@H](O)[C@@H]1O] |
| 50 | 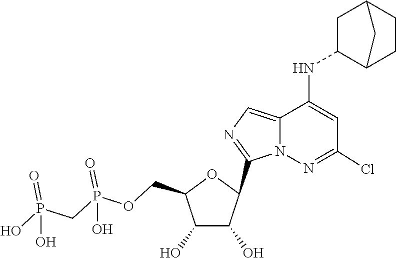 |
| 51 | 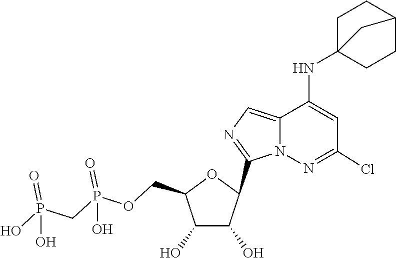 [SMILES: O=P(O)(O)CP(=O)(O)OC[C@H]1O[C@@H](c2ncc3c(NC45CCC(CC4)C5)cc(Cl)nn23)[C@H](O)[C@@H]1O] |
| 52 | 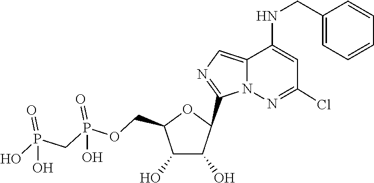 [SMILES: O=P(O)(O)CP(=O)(O)OC[C@H]1O[C@@H](c2ncc3c(NCc4ccccc4)cc(Cl)nn23)[C@H](O)[C@@H]1O] |
| 53 | 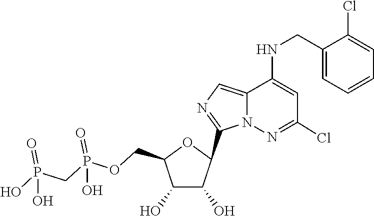 [SMILES: O=P(O)(O)CP(=O)(O)OC[C@H]1O[C@@H](c2ncc3c(NCc4ccccc4Cl)cc(Cl)nn23)[C@H](O)[C@@H]1O] |
| 54 | 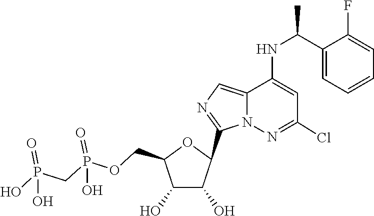 [SMILES: C[C@H](Nc1cc(Cl)nn2c([C@@H]3O[C@H](COP(=O)(O)CP(=O)(O)O)[C@@H](O)[C@H]3O)ncc12)c1ccccc1F] |
| 55 | 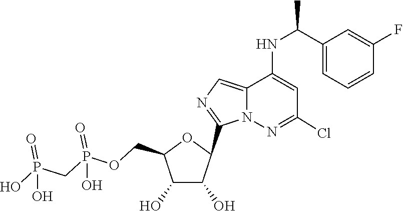 [SMILES: C[C@H](Nc1cc(Cl)nn2c([C@@H]3O[C@H](COP(=O)(O)CP(=O)(O)O)[C@@H](O)[C@H]3O)ncc12)c1cccc(F)c1] |
| 56 | 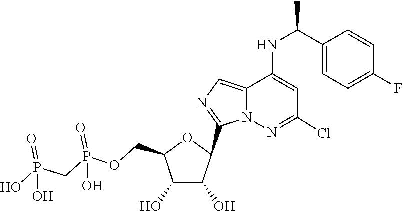 [SMILES: C[C@H](Nc1cc(Cl)nn2c([C@@H]3O[C@H](COP(=O)(O)CP(=O)(O)O)[C@@H](O)[C@H]3O)ncc12)c1ccc(F)cc1] |
| 57 | 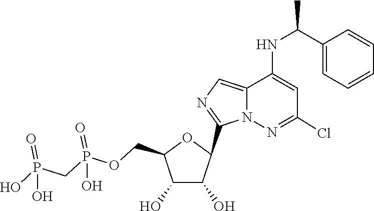 [SMILES: C[C@H](Nc1cc(Cl)nn2c([C@@H]3O[C@H](COP(=O)(O)CP(=O)(O)O)[C@@H](O)[C@H]3O)ncc12)c1ccccc1] |
| 58 | 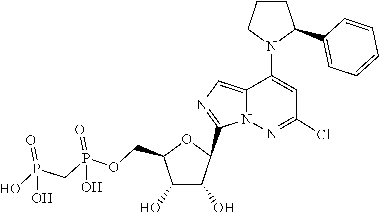 [SMILES: O=P(O)(O)CP(=O)(O)OC[C@H]1O[C@@H](c2ncc3c(N4CCC[C@H]4c4ccccc4)cc(Cl)nn23)[C@H](O)[C@@H]1O] |
| 59 | 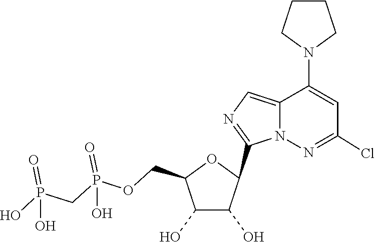 [SMILES: O=P(O)(O)CP(=O)(O)OC[C@H]1O[C@@H](c2ncc3c(N4CCCC4)cc(Cl)nn23)[C@H](O)[C@@H]1O] |
| 60 | 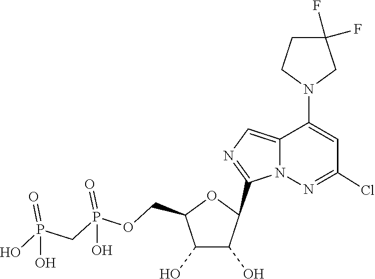 [SMILES: O=P(O)(O)CP(=O)(O)OC[C@H]1O[C@@H](c2ncc3c(N4CCC(F)(F)C4)cc(Cl)nn23)[C@H](O)[C@@H]1O] |
| 61 | 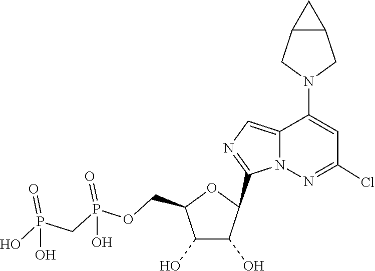 [SMILES: O=P(O)(O)CP(=O)(O)OC[C@H]1O[C@@H](c2ncc3c(N4CC5CC5C4)cc(Cl)nn23)[C@H](O)[C@@H]1O] |
| 62 | 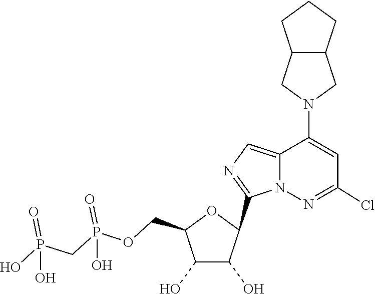 [SMILES: O=P(O)(O)CP(=O)(O)OC[C@H]1O[C@@H](c2ncc3c(N4CC5CCCC5C4)cc(Cl)nn23)[C@H](O)[C@@H]1O] |
| 63 | 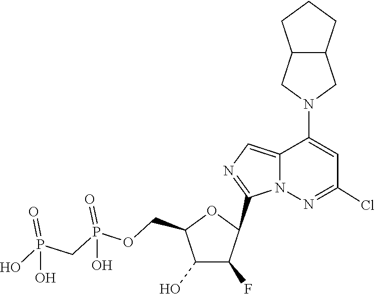 [SMILES: O=P(O)(O)CP(=O)(O)OC[C@H]1O[C@@H](c2ncc3c(N4CC5CCCC5C4)cc(Cl)nn23)[C@@H](F)[C@@H]1O] |
| 64 | 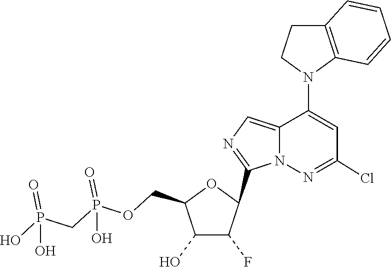 [SMILES: O=P(O)(O)CP(=O)(O)OC[C@H]1O[C@@H](c2ncc3c(N4CCc5ccccc54)cc(Cl)nn23)[C@H](F)[C@@H]1O] |
| 65 | 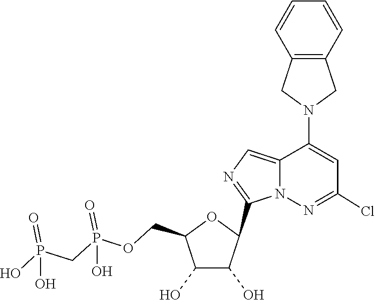 [SMILES: O=P(O)(O)CP(=O)(O)OC[C@H]1O[C@@H](c2ncc3c(N4Cc5ccccc5C4)cc(Cl)nn23)[C@H](O)[C@@H]1O] |
| 66 | 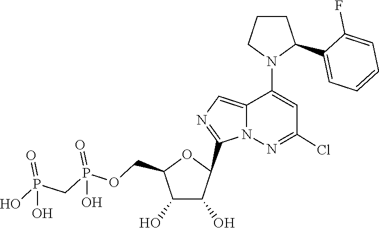 [SMILES: O=P(O)(O)CP(=O)(O)OC[C@H]1O[C@@H](c2ncc3c(N4CCC[C@H]4c4ccccc4F)cc(Cl)nn23)[C@H](O)[C@@H]1O] |
| 67 | 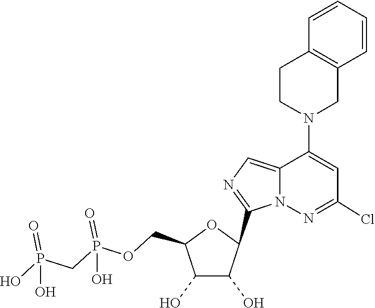 [SMILES: O=P(O)(O)CP(=O)(O)OC[C@H]1O[C@@H](c2ncc3c(N4CCc5ccccc5C4)cc(Cl)nn23)[C@H](O)[C@@H]1O] |
| 68 | 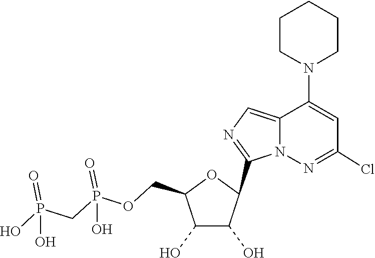 [SMILES: O=P(O)(O)CP(=O)(O)OC[C@H]1O[C@@H](c2ncc3c(N4CCCCC4)cc(Cl)nn23)[C@H](O)[C@@H]1O] |
| 69 | 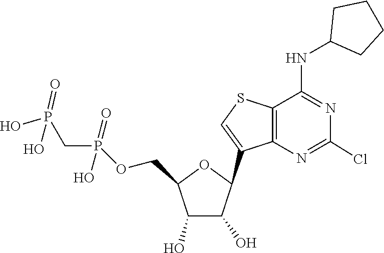 [SMILES: O=P(O)(O)CP(=O)(O)OC[C@H]1O[C@@H](c2csc3c(NC4CCCC4)nc(Cl)nc23)[C@H](O)[C@@H]1O] |
| 70 | 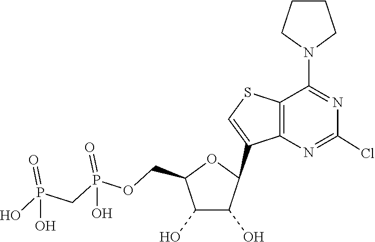 [SMILES: O=P(O)(O)CP(=O)(O)OC[C@H]1O[C@@H](c2csc3c(N4CCCC4)nc(Cl)nc23)[C@H](O)[C@@H]1O] |
| 71 | 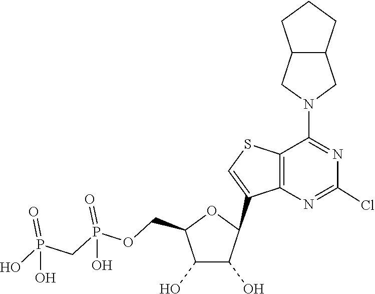 [SMILES: O=P(O)(O)CP(=O)(O)OC[C@H]1O[C@@H](c2csc3c(N4CC5CCCC5C4)nc(Cl)nc23)[C@H](O)[C@@H]1O] |
| 72 | 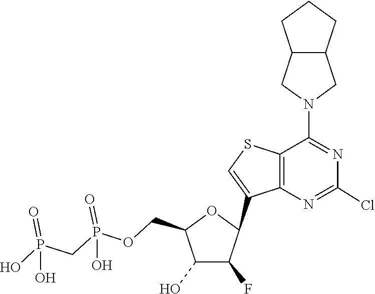 [SMILES: O=P(O)(O)CP(=O)(O)OC[C@H]1O[C@@H](c2csc3c(N4CC5CCCC5C4)nc(Cl)nc23)[C@@H](F)[C@@H]1O] |
| 73 | 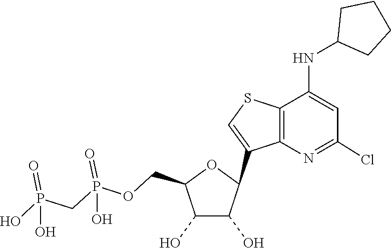 [SMILES: O=P(O)(O)CP(=O)(O)OC[C@H]1O[C@@H](c2csc3c(NC4CCCC4)cc(Cl)nc23)[C@H](O)[C@@H]1O] |
| 74 | 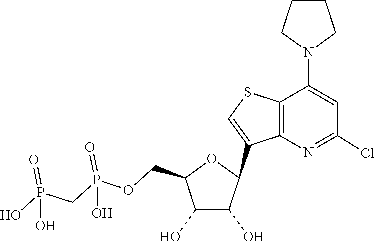 [SMILES: O=P(O)(O)CP(=O)(O)OC[C@H]1O[C@@H](c2csc3c(N4CCCC4)cc(Cl)nc23)[C@H](O)[C@@H]1O] |
| 75 | 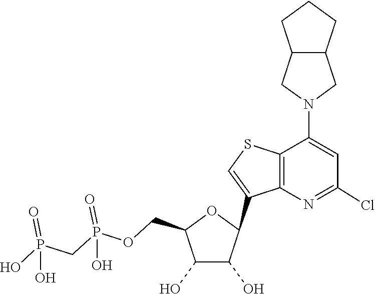 [SMILES: O=P(O)(O)CP(=O)(O)OC[C@H]1O[C@@H](c2csc3c(N4CC5CCCC5C4)cc(Cl)nc23)[C@H](O)[C@@H]1O] |
| 76 | 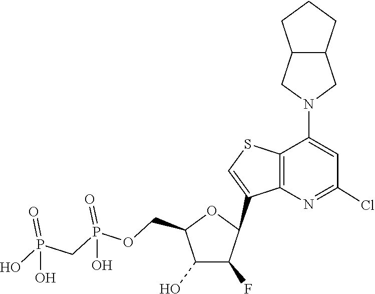 [SMILES: O=P(O)(O)CP(=O)(O)OC[C@H]1O[C@@H](c2csc3c(N4CC5CCCC5C4)cc(Cl)nc23)[C@@H](F)[C@@H]1O] |
| 77 | 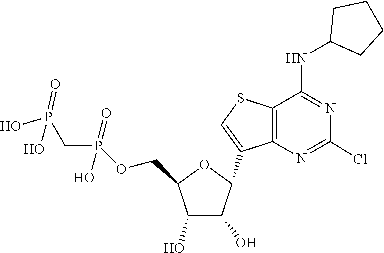 [SMILES: O=P(O)(O)CP(=O)(O)OC[C@H]1O[C@H](c2csc3c(NC4CCCC4)nc(Cl)nc23)[C@H](O)[C@@H]1O] |

Also provided are salts of compounds disclosed herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds described herein include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds described herein can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound, such as would be generated in vivo following administration to a human.

Solvates of a compound provided herein or a salt thereof are also contemplated. Solvates contain either stoichiometric or non-stoichiometric amounts of a solvent and are often formed during the process of crystallization. Hydrates are formed when the solvent is water, or alcoholates are formed when the solvent is alcohol.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### Pharmaceutical Compositions and Formulations

Pharmaceutical compositions of any of the compounds detailed herein are embraced by this disclosure. Thus, the present disclosure includes pharmaceutical compositions comprising a compound as detailed herein, or a stereoisomer, tautomer, prodrug, or a pharmaceutically acceptable salt of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or a stereoisomer, tautomer, prodrug, or a pharmaceutically acceptable salt of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a stereoisomer, tautomer, prodrug, or a pharmaceutically acceptable salt of any of the foregoing, can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

A compound detailed herein, or a stereoisomer, tautomer, prodrug, or a pharmaceutically acceptable salt of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use

Compounds and compositions detailed herein, such as a pharmaceutical composition containing a compound of any formula provided herein, or a stereoisomer, tautomer, prodrug, or a pharmaceutically acceptable salt of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In some embodiments, provided herein is a method of treating a disease or disorder mediated by CD73 in an individual in need thereof, comprising administering to the individual a therapeutically effective amount of a compound or a composition disclosed herein. In some embodiments, the compound or a pharmaceutically acceptable salt thereof, or the composition is administered to the individual according to a dosage and/or method of administration described herein. In some embodiments, the method further comprises administering to the individual an additional therapeutic agent. In some embodiments, the additional therapeutic agent is an immune checkpoint inhibitor, a chemotherapeutic agent, an immune-modulating agent, an inflammation-modulating agent, or an anti-infective agent. In some embodiments, the additional therapeutic agent is an immune checkpoint inhibitor. In some embodiments, the checkpoint inhibitor comprises a cytotoxic T lymphocyte associated protein 4 (CTLA-4) inhibitor, programmed cell death protein 1 (PD-1) inhibitor, or programmed death ligand 1 (PD-L1) inhibitor. In some embodiments, the checkpoint inhibitor comprises a CTLA-4 inhibitor such as ipilimumab. In some embodiments, the checkpoint inhibitor comprises a PD-1 inhibitor such as nivolumab or pembrolizumab. In some embodiments, the checkpoint inhibitor comprises a PD-L1 inhibitor such as atezolizumab. In some embodiments, the additional therapeutic agent is a chemotherapeutic agent. In some embodiments, the additional therapeutic agent is an immune-modulating agent. In some embodiments, the additional therapeutic agent is an inflammation-modulating agent. In some embodiments, the additional therapeutic agent is an anti-infective agent. Also provided herein is the use of a compound or a composition disclosed herein for the preparation of a medicament for treating a disease or disorder mediated by CD73.

Compounds and compositions detailed herein can inhibit the activity of the CD73. For example, a compound or a composition disclosed herein can be used to inhibit activity of CD73 in a cell or in an individual or patient in need of inhibition of the enzyme by administering an inhibiting amount of the compound or the composition to the cell, individual, or patient. In some embodiments, provided is a method of reversing or stopping the progression of CD73-mediated immunosuppression in an individual, comprising administering to the individual a therapeutically effective amount of a compound or a composition disclosed herein. In some embodiments, provided is a method of inhibiting CD73-catalyzed hydrolysis of adenosine monophosphate, comprising administering to the individual a therapeutically effective amount of a compound or a composition disclosed herein.

Compounds and compositions detailed herein are useful in the treatment of cancer. Examples of cancers include, without limitation, bladder cancer, leukemia, glioma, glioblastoma, melanoma, ovarian cancer, thyroid cancer, esophageal cancer, prostate cancer, lung cancer, colorectal cancer, pancreatic cancer, skin cancer, liver cancer, gastric cancer, head & neck cancer, and breast cancer. In some embodiments, provided herein is a method of treating cancer in an individual in need thereof, comprising administering a therapeutically effective amount of a compound or a composition disclosed herein. Also provided is use of a compound or a composition disclosed herein for the preparation of a medicament for treating cancer.

Compounds and compositions detailed herein are useful in the treatment of immune-related disease. The term “immune-related disease” means a disease in which a component of the immune system causes, mediates or otherwise contributes to a morbidity. Also included are diseases in which stimulation or intervention of the immune response has an ameliorative effect on progression of the disease. Examples of immune-related diseases include, without limitation, immune-mediated inflammatory diseases, infectious diseases, immunodeficiency diseases, and neoplasia, etc.

### Combinations

In certain aspects, compounds or compositions described herein are administered to an individual for treatment of a disease in combination with one or more additional pharmaceutical agents that can treat the disease. For example, in some embodiments, an effective amount of a compound or a composition disclosed herein is administered to an individual for the treatment of a disease such as cancer in combination with an additional therapeutic agent. In some embodiments, the additional therapeutic agent is an immune checkpoint inhibitor, a chemotherapeutic agent, an immune-modulating agent, an inflammation-modulating agent, or an anti-infective agent. In some embodiments, the additional therapeutic agent is an immune checkpoint inhibitor. In some embodiments, the checkpoint inhibitor comprises a CTLA-4 inhibitor, PD-1 inhibitor, or PD-L1 inhibitor. In some embodiments, the checkpoint inhibitor comprises a CTLA-4 inhibitor such as ipilimumab. In some embodiments, the checkpoint inhibitor comprises a PD-1 inhibitor such as nivolumab or pembrolizumab. In some embodiments, the checkpoint inhibitor comprises a PD-L1 inhibitor such as atezolizumab. In some embodiments, the additional therapeutic agent is a chemotherapeutic agent. In some embodiments, the additional therapeutic agent is a chemotherapeutic agent. In some embodiments, the additional therapeutic agent is an immune-modulating agent. In some embodiments, the additional therapeutic agent is an inflammation-modulating agent. In some embodiments, the additional therapeutic agent is an anti-infective agent.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

Certain exemplified embodiments are provided below.

Embodiments 1. A compound of formula (I):

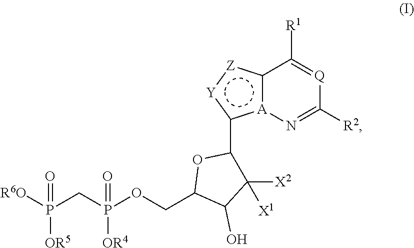

or a stereoisomer, tautomer, prodrug, or a pharmaceutically acceptable salt of any of the foregoing, wherein:

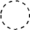

indicates a fully saturated, partially saturated, or aromatic ring;
X¹ and X² are each independently H, —CN, C₁₋₆ alkyl, —OR′, or halogen, wherein R′ is H, C₁₋₆ alkyl, C₃₋₁₂cycloalkyl, 3- to 12-membered heterocyclyl, 5- to 10-membered heteroaryl, or C₆₋₁₄ aryl;

### Q is N or CR³;

### Y is CH or N;

Z is CH, O, S, or N, provided that,

when Z is O, S, or N, then Y is CH,

when Z is CH, then Y is N, and

when Z is CH, O, or N, then Q is CR³;

### A is C or N;

R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl of R^{1a} and R^{1b} are each independently optionally substituted with R⁷, or

R^{1a} and R^{1b} are taken together with the nitrogen atom to which they attach to form a 3- to 12-membered heterocyclyl optionally substituted with C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, C₆₋₁₄ aryl, halogen, hydroxyl, C₁₋₆ alkoxy, or —CN, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently substituted with C₁₋₆ alkyl, halogen, hydroxyl, C₁₋₆ alkoxy, or —CN;

R² is H, C₁₋₆ alkyl, C₂₋₆ alkenyl, C₂₋₆ alkynyl, halogen, —CN, —NR^{2a}R^{2b}, —OR²a, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl of R² are each independently optionally substituted with R⁸, and wherein:

R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   R³ is H, C₁₋₆ alkyl, C₁₋₆ haloalkyl, halogen, or —CN;
   R⁴, R⁵, and R⁶ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
   each R⁷ is independently oxo, C₁₋₆ alkyl, C₂₋₆ alkenyl, C₂₋₆ alkynyl, halogen, —CN, —OR^{7a}, —SR^{7a}, —NR^{7a}R^{7b}, —NO₂, —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃. 6 cycloalkyl, 3- to 12-membered heterocyclyl, 5- to 10-membered heteroaryl, or C₆₋₁₄ aryl, wherein the C₁₋₆ alkyl, C₃₋₆ cycloalkyl, 3- to 12-membered heterocyclyl, 5- to 10-membered heteroaryl, and C₆₋₁₄ aryl of R⁷ are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   each R⁸ is independently oxo, C₁₋₆ alkyl, C₂₋₆ alkenyl, C₂₋₆ alkynyl, halogen, —CN, —OR^{8a}, —SR^{8a}, —NR^{8a}R^{8b}, —NO₂, —C═NH(OR^{8a}), —C(O)R^{8a}, —OC(O)R^{8a}, —C(O)OR^{8a}, —C(O)NR^{8a}R^{8b}, —OC(O)NR^{8a}R^{8b}, —NR^{8a}C(O)R^{8b}, —NR^{8a}C(O)OR^{8b}, —S(O)R^{8a}, —S(O)₂R^{8a}, —NR^{8a}S(O)R^{8b}, —C(O)NR^{8a}S(O)R^{8b}, —NR^{8a}S(O)₂R^{8b}, —C(O)NR^{8a}S(O)₂R^{8b}, —S(O)NR^{8a}R^{8b}, —S(O)₂NR^{8a}R^{8b}, —P(O)(OR^{8a}) (OR^{8b}), C₃₋₆ cycloalkyl, 3- to 12-membered heterocyclyl, 5- to 10-membered heteroaryl, or C₆₋₁₄ aryl, wherein:

R^{8a} and R^{8b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{8a} and R^{8b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.
   Embodiments 2. The compound of embodiment 1, or a stereoisomer, tautomer, prodrug, or a pharmaceutically acceptable salt of any of the foregoing, wherein Z is CH.
   Embodiments 3. The compound of embodiment 1, or a stereoisomer, tautomer, prodrug, or a pharmaceutically acceptable salt of any of the foregoing, wherein Z is O.
   Embodiments 4. The compound of embodiment 1, or a stereoisomer, tautomer, prodrug, or a pharmaceutically acceptable salt of any of the foregoing, wherein Z is N.
   Embodiments 5. The compound of embodiment 1, or a stereoisomer, tautomer, prodrug, or a pharmaceutically acceptable salt of any of the foregoing, wherein Z is S.
   Embodiments 6. The compound of any one of embodiments 1-5, or a stereoisomer, tautomer, prodrug, or a pharmaceutically acceptable salt of any of the foregoing, wherein A is N.
   Embodiments 7. The compound of any one of embodiments 1-5, or a stereoisomer, tautomer, prodrug, or a pharmaceutically acceptable salt of any of the foregoing, wherein A is C.
   Embodiments 8. The compound of any one of embodiments 1-7, or a stereoisomer, tautomer, prodrug, or a pharmaceutically acceptable salt of any of the foregoing, wherein Q is CR³.
   Embodiments 9. The compound of any one of embodiments 5-7, or a stereoisomer, tautomer, prodrug, or a pharmaceutically acceptable salt of any of the foregoing, wherein Q is N.
   Embodiments 10. The compound of embodiment 1, or a stereoisomer, tautomer, prodrug, or a pharmaceutically acceptable salt of any of the foregoing, wherein the compound is of formula (II):

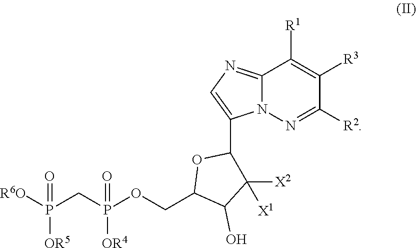

Embodiments 11. The compound of embodiment 1, or a stereoisomer, tautomer, prodrug, or a pharmaceutically acceptable salt of any of the foregoing, wherein the compound is of formula (III):

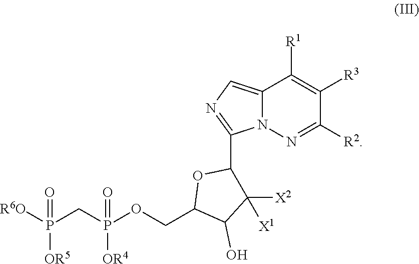

Embodiments 12. The compound of embodiment 1, or a stereoisomer, tautomer, prodrug, or a pharmaceutically acceptable salt of any of the foregoing, wherein the compound is of formula (IV):

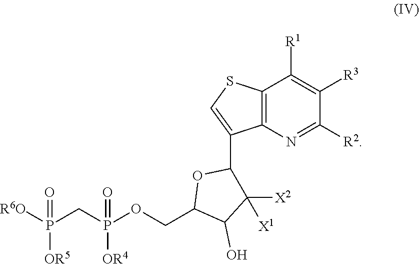

Embodiments 13. The compound of embodiment 1, or a stereoisomer, tautomer, prodrug, or a pharmaceutically acceptable salt of any of the foregoing, wherein the compound is of formula (V):

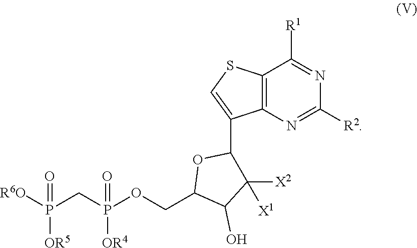

Embodiments 14. The compound of any one of embodiments 1-13, or a stereoisomer, tautomer, prodrug, or a pharmaceutically acceptable salt of any of the foregoing, wherein X is H or —OH.
Embodiments 15. The compound of any one of embodiments 1-14, or a stereoisomer, tautomer, prodrug, or a pharmaceutically acceptable salt of any of the foregoing, wherein X² is H or halogen.
Embodiments 16. The compound of any one of embodiments 1-15, or a stereoisomer, tautomer, prodrug, or a pharmaceutically acceptable salt of any of the foregoing, wherein R¹ is —NR^{1a}R^{1b}.
Embodiments 17. The compound of any one of embodiments 1-15, or a stereoisomer, tautomer, prodrug, or a pharmaceutically acceptable salt of any of the foregoing, wherein R¹ is —OR^{1a}.
Embodiments 18. The compound of any one of embodiments 1-17, or a stereoisomer, tautomer, prodrug, or a pharmaceutically acceptable salt of any of the foregoing, wherein R^{1a} is C₁₋₆ alkyl, C₃₋₁₂ cycloalkyl, or 3- to 12-membered heterocyclyl, each of which is independently optionally substituted with R⁷.
Embodiments 19. The compound of any one of embodiments 1-17, or a stereoisomer, tautomer, prodrug, or a pharmaceutically acceptable salt of any of the foregoing, wherein R^{1a} is

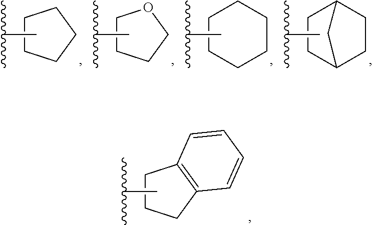

methyl, or ethyl, each of which is optionally substituted with R⁷.
Embodiments 20. The compound of embodiment 18 or 19, or a stereoisomer, tautomer, prodrug, or a pharmaceutically acceptable salt of any of the foregoing, wherein R⁷ is halogen or phenyl optionally substituted with halogen.
Embodiments 21. The compound of any one of embodiments 1-17, or a stereoisomer, tautomer, prodrug, or a pharmaceutically acceptable salt of any of the foregoing, wherein R^{1a} is

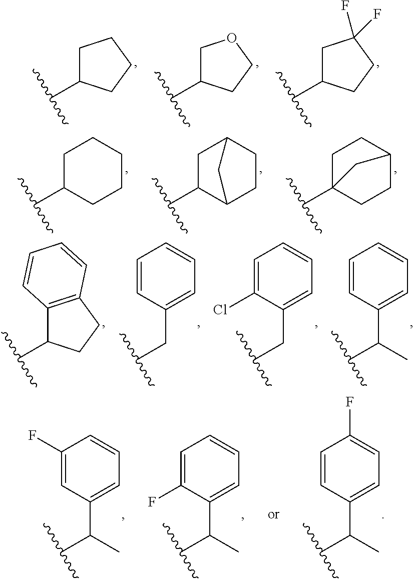

Embodiments 22. The compound of any one of embodiments 1-16 and 18-21, or a stereoisomer, tautomer, prodrug, or a pharmaceutically acceptable salt of any of the foregoing, wherein R^{1b} is H or C₁₋₆ alkyl.
Embodiments 23. The compound of any one of embodiments 1-16, or a stereoisomer, tautomer, prodrug, or a pharmaceutically acceptable salt of any of the foregoing, wherein R^{1a} and R^{1b} are taken together with the nitrogen atom to which they attach to form a 3- to 12-membered heterocyclyl optionally substituted with C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, C₆₋₁₄ aryl, halogen, hydroxyl, C₁₋₆ alkoxy, or —CN, wherein the C₁₋₆ alkyl, C₂₋₆ alkenyl, C₂₋₆ alkynyl, C₃-12 cycloalkyl, 3- to 12-membered heterocyclyl, 5- to 10-membered heteroaryl, and C₆₋₁₄ aryl are each independently substituted with C₁₋₆ alkyl, halogen, hydroxyl, C₁₋₆ alkoxy, or —CN.
Embodiments 24. The compound of embodiment 23, or a stereoisomer, tautomer, prodrug, or a pharmaceutically acceptable salt of any of the foregoing, wherein R^{1a} and R^{1b} are taken together with the nitrogen atom to which they attach to form a moiety selected from the group consisting of:

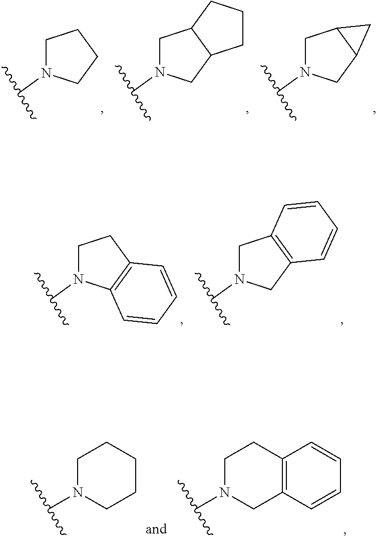

each of which is optionally substituted with halogen or phenyl optionally substituted with halogen.
Embodiments 25. The compound of embodiment 24, or a stereoisomer, tautomer, prodrug, or a pharmaceutically acceptable salt of any of the foregoing, wherein R^{1a} and R^{1b} are taken together with the nitrogen atom to which they attach to form a moiety selected from the group consisting of:

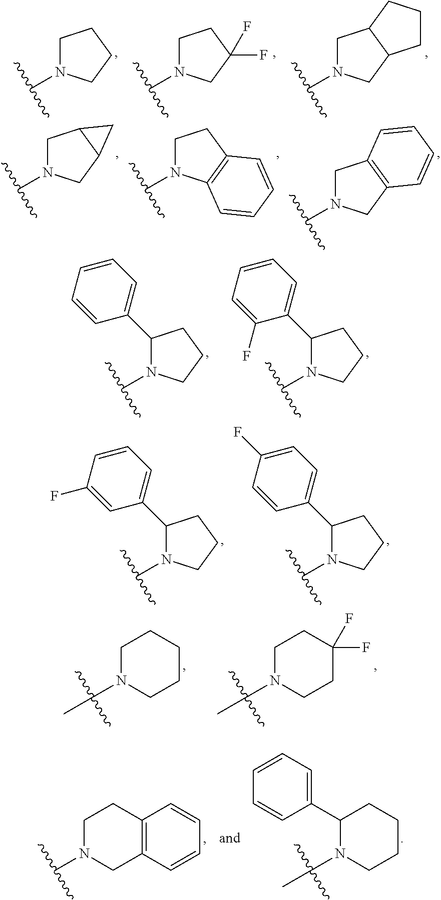

Embodiments 26. The compound of any one of embodiments 1-25, or a stereoisomer, tautomer, prodrug, or a pharmaceutically acceptable salt of any of the foregoing, wherein R¹ is selected from the group consisting of

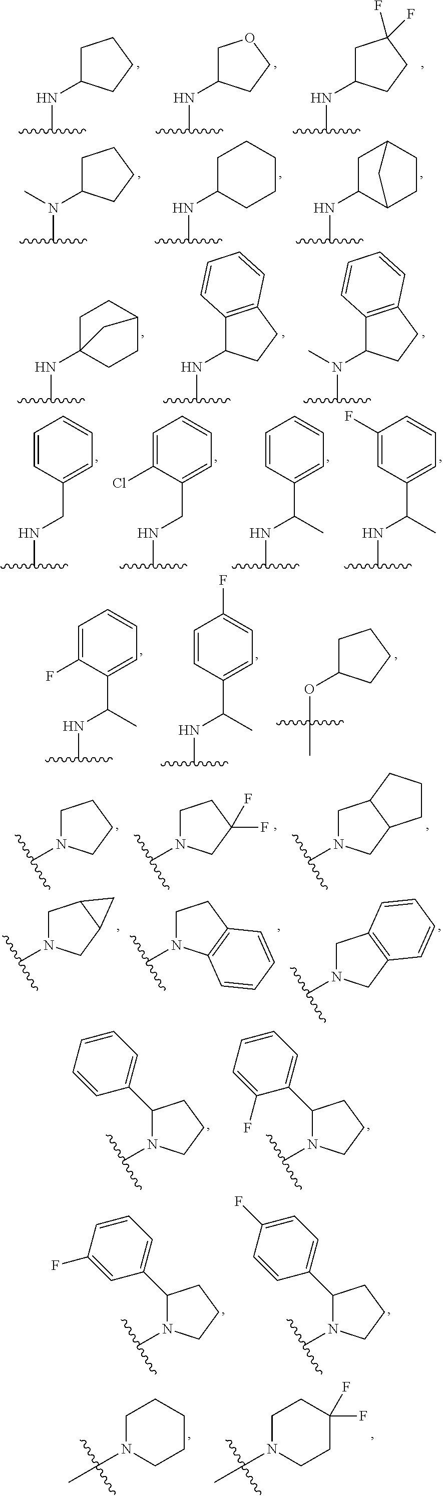

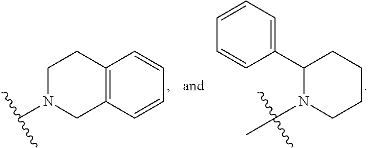 [SMILES: *N1CCc2ccccc2C1 ; *N1CCCCC1c1ccccc1]

Embodiments 27. The compound of any one of embodiments 1-26, or a stereoisomer, tautomer, prodrug, or a pharmaceutically acceptable salt of any of the foregoing, wherein R² is H, —CN, or halogen.
Embodiments 28. The compound of any one of embodiments 1-27, or a stereoisomer, tautomer, prodrug, or a pharmaceutically acceptable salt of any of the foregoing, wherein R³ is H.
Embodiments 29. The compound of any one of embodiments 1-28, or a stereoisomer, tautomer, prodrug, or a pharmaceutically acceptable salt of any of the foregoing, wherein R⁴ is H.
Embodiments 30. The compound of any one of embodiments 1-29, or a stereoisomer, tautomer, prodrug, or a pharmaceutically acceptable salt of any of the foregoing, wherein R⁵ is H.
Embodiments 31. The compound of any one of embodiments 1-30, or a stereoisomer, tautomer, prodrug, or a pharmaceutically acceptable salt of any of the foregoing, wherein R⁶ is H.
Embodiments 32. A compound selected from the group consisting of the compounds in Table 1, or a stereoisomer, tautomer, prodrug, or a pharmaceutically acceptable salt of any of the foregoing.
Embodiments 33. A pharmaceutical composition comprising the compound of any one of embodiments 1-32, or a stereoisomer, tautomer, prodrug, or a pharmaceutically acceptable salt of any of the foregoing, and a pharmaceutically acceptable excipient.
Embodiments 34. A kit comprising the compound of any one of embodiments 1-32, or a stereoisomer, tautomer, prodrug, or a pharmaceutically acceptable salt of any of the foregoing.
Embodiments 35. A method of treating a disease mediated by CD73 in an individual in need thereof, comprising administering to the individual a therapeutically effective amount of the compound of any one of embodiments 1-32, or a stereoisomer, tautomer, prodrug, or a pharmaceutically acceptable salt of any of the foregoing.
Embodiments 36. The method of embodiment 35, wherein the disease is cancer.
Embodiments 37. The method of embodiment 35 or 36, further comprising administering to the individual an additional therapeutic agent, wherein the additional therapeutic agent is an immune checkpoint inhibitor, a chemotherapeutic agent, an immune-modulating agent, an inflammation-modulating agent, or an anti-infective agent.
Embodiments 38. The method of embodiment 37, wherein the additional therapeutic agent is an immune checkpoint inhibitor.
Embodiments 39. The method of embodiment 38, wherein the additional therapeutic agent is a cytotoxic T lymphocyte associated protein 4 (CTLA-4) inhibitor, a programmed cell death protein 1 (PD-1) inhibitor, or a programmed death ligand 1 (PD-L1) inhibitor.
Embodiments 40. A method of reversing or stopping the progression of CD73-mediated immunosuppression in an individual, comprising administering to the individual a therapeutically effective amount of the compound of any one of embodiments 1-32, or a stereoisomer, tautomer, prodrug, or a pharmaceutically acceptable salt of any of the foregoing.
Embodiments 41. A method of inhibiting CD73-catalyzed hydrolysis of adenosine monophosphate, comprising contacting CD73 with the compound of any one of embodiments 1-32, or a stereoisomer, tautomer, prodrug, or a pharmaceutically acceptable salt of any of the foregoing.
Embodiments 42. Use of the compound of any one of embodiments 1-32, or a stereoisomer, tautomer, prodrug, or a pharmaceutically acceptable salt of any of the foregoing, in the manufacture of a medicament for use in therapy.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates of a compound provided herein or a salt thereof are also contemplated. Solvates contain either stoichiometric or non-stoichiometric amounts of a solvent and are often formed during the process of crystallization. Hydrates are formed when the solvent is water, or alcoholates are formed when the solvent is alcohol.

Chromatography, recrystallization and other conventional separation procedures may also be used with intermediates or final products where it is desired to obtain a particular isomer of a compound or to otherwise purify a product of a reaction.

General methods of preparing compounds according to the present disclosure are depicted in the schemes below, wherein PG is a protecting group; and X¹, X², A, Y, Z, Q, R¹, R², R^{1a}, R^{1b}, R⁴, R⁵, and R⁶ are as detailed herein.

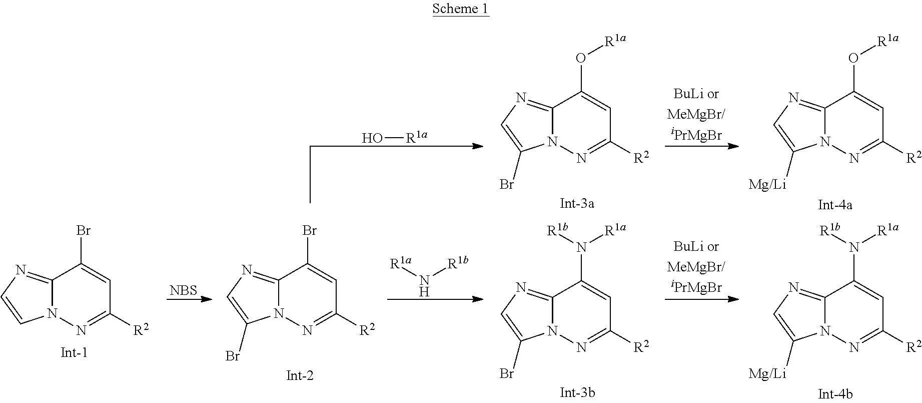

As shown in Scheme 1, some compounds of this invention can be prepared from Int-1. Int-1 is commercially available or can be prepared by procedures described in the literature. For example, the intermediate where R²=Cl, can be synthesized according to a procedure given in J. Med. Chem., 55, 10414-10423, (2012). Compound Int-1 can be converted into Int-2 by reacting with NBS in an appropriate solvent such as, for example, CHCl₃ or CCl₄. Int-2 can be reacted with an alcohol in the presence of a base, such as sodium hydride in a solvent, such as THF, to give Int-3a. Alternatively, Int-2 can be reacted with a primary or secondary amine in the presence of a base, such as, for example, Et₃N or DIEA in a solvent, such as THF or EtOH to give Int-3b. Int-3a and Int-3b can be treated with an organometallic compound to give the metalated species Int-4a or Int-4b, respectively. This halogen-metal exchange of Int-3a to give Int-4a and Int-3b to give Int-4b, respectively, can be accomplished, for example, with n-BuLi, sec-BuLi or tert-Buli or with MeMgBr and iPrMgBr in a solvent such as diethylether, dimethoxyethane, or THF.

The organometallic species Int-4a or Int-4b can be added to the appropriately protected lactone, Int-5a, to give Int-6a, as shown in Scheme 2. The organometallic species Int-4a or Int-4b can also be added to appropriately protected lactone, Int-5b, to give Int-6b, as shown in Scheme 2. Appropriate protecting groups (PG) are known to those skilled in the art and are described, for example, in “Greene's Protective Groups in Organic Synthesis”, John Wiley & Sons, Inc., 2014. Int-6a or Int-6b can be reduced to Int-7a or Int-7b, respectively, with a silane in presence of a Lewis acid. For example, Et₃SiH in presence of BF₃—OEt₂ will accomplish this reaction. Int-7a and Int-7b can be deprotected to give Int-8a or Int-8b, respectively. Deprotection will be accomplished by methods known to the skilled practitioner and are also described in “Greene's Protective Groups in Organic Synthesis”, John Wiley & Sons, Inc., 2014. For example, if the protection group is a benzyl ether (PG=Bn), hydrogen in presence of a catalyst, such as Pd on carbon, or BCl₃ in DCM will achieve the deprotection. If the protection group is a silyl ether, the deprotection can be accomplished, for example, by using Bu₄NF in THF. Many other protecting groups and methods for removing them are known to those skilled in the art.

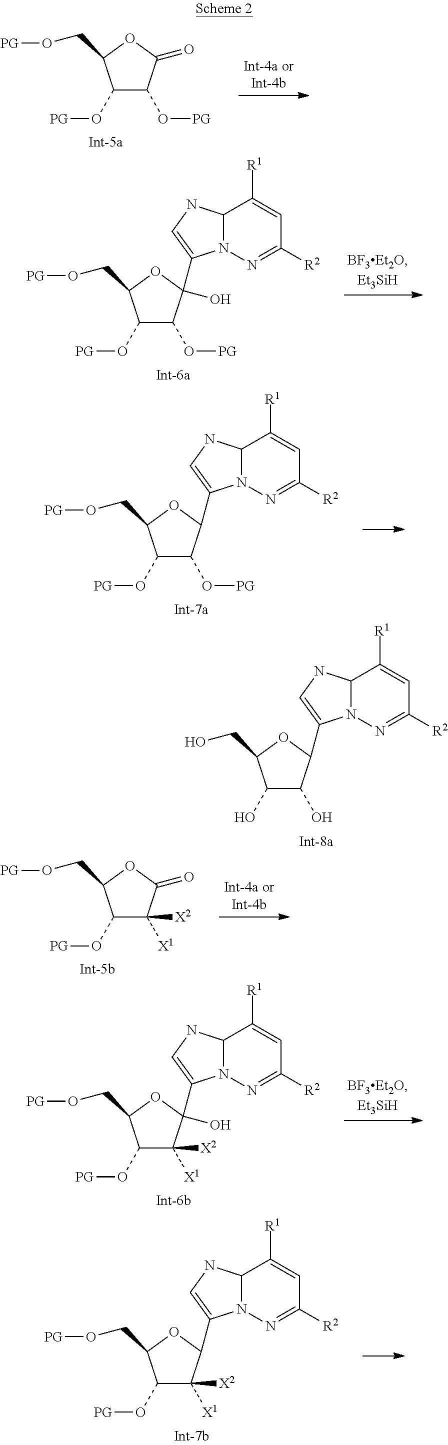

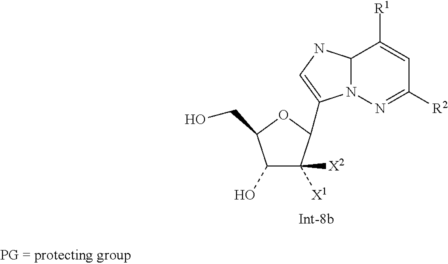

As shown in Scheme 3, Int-8a or Int-8b can be converted into the ((hydroxy-phosphoryl)methyl)phosphonic acid A by reacting with, for example, methylenebis(phosphonic dichloride) in trimethylphosphate followed by hydrolysis in presence of a base, such as, TEAC. Alternatively, Int-8a can be converted into Int-9. The primary alcohol Int-9 can be then be condensed with di-tert-butyl ((tert-butoxy(diisopropylamino)phosphaneyl)methyl)phosphonate, followed by in situ oxidation to provide the methylene bisphosphonate tetraester according to a procedure similar to the one described in Angew. Chem., Int. Ed., 56, 2955-2959, (2017). Deprotection of the tetraester gives the methylene bisphosphonate A.

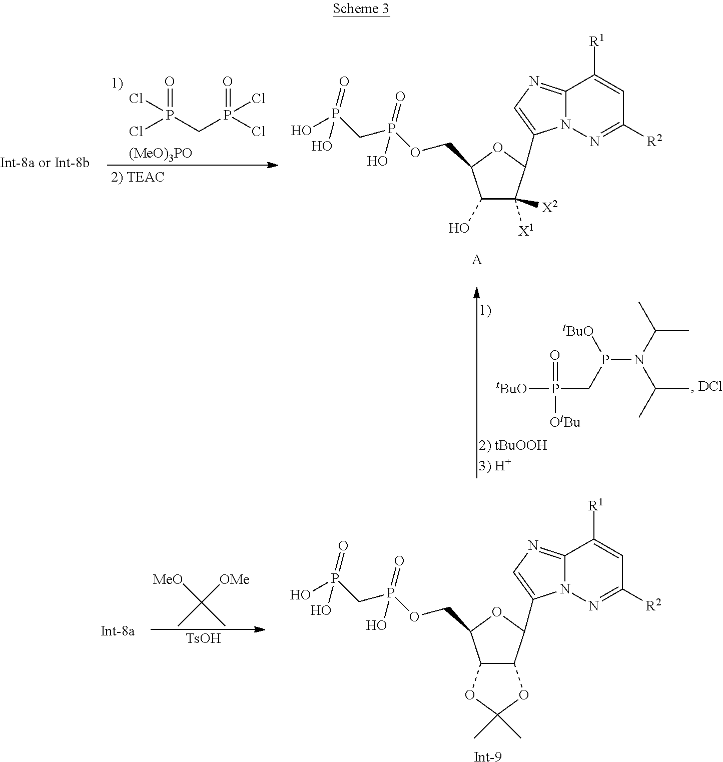

Other compounds of this invention can be prepared as shown in Schemes 4 and 5. Scheme 4 shows an exemplary synthesis of Int-12. Treating the lactone Int-5 with a reducing agent, such as DIBAL-H in a solvent, such as toluene, will give the lactol Int-10a. Other reducing agents, such as lithium triethylborohydride or sodium borohydride will also accomplish this transformation [see e.g. Carbohydr. Res. 432, 17, (2016)].

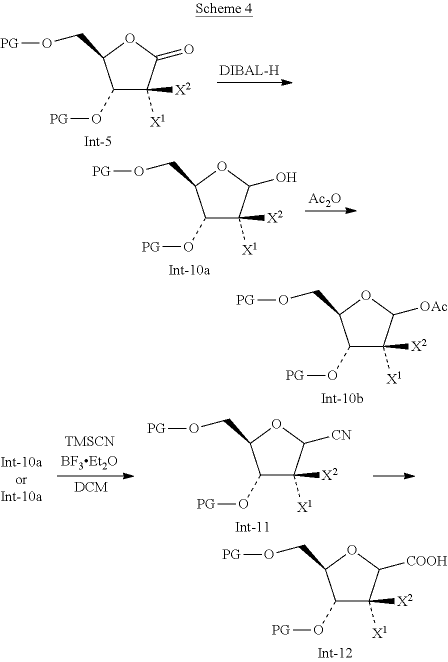

The conversion of the lactol Int-10 to the acetate Int-10b is achieved by acetylation with acetic anhydride. Treatment of either Int-10a or Int-10b with trimethylsilyl cyanide in an inert solvent, such as DCM, in the presence of a Lewis acid, such as boron trifluoride etherate or InBr₃ will give Int-11. Int-11 can be converted to the acid Int-12 by hydrolysis with, for example, AcOH and HCl.

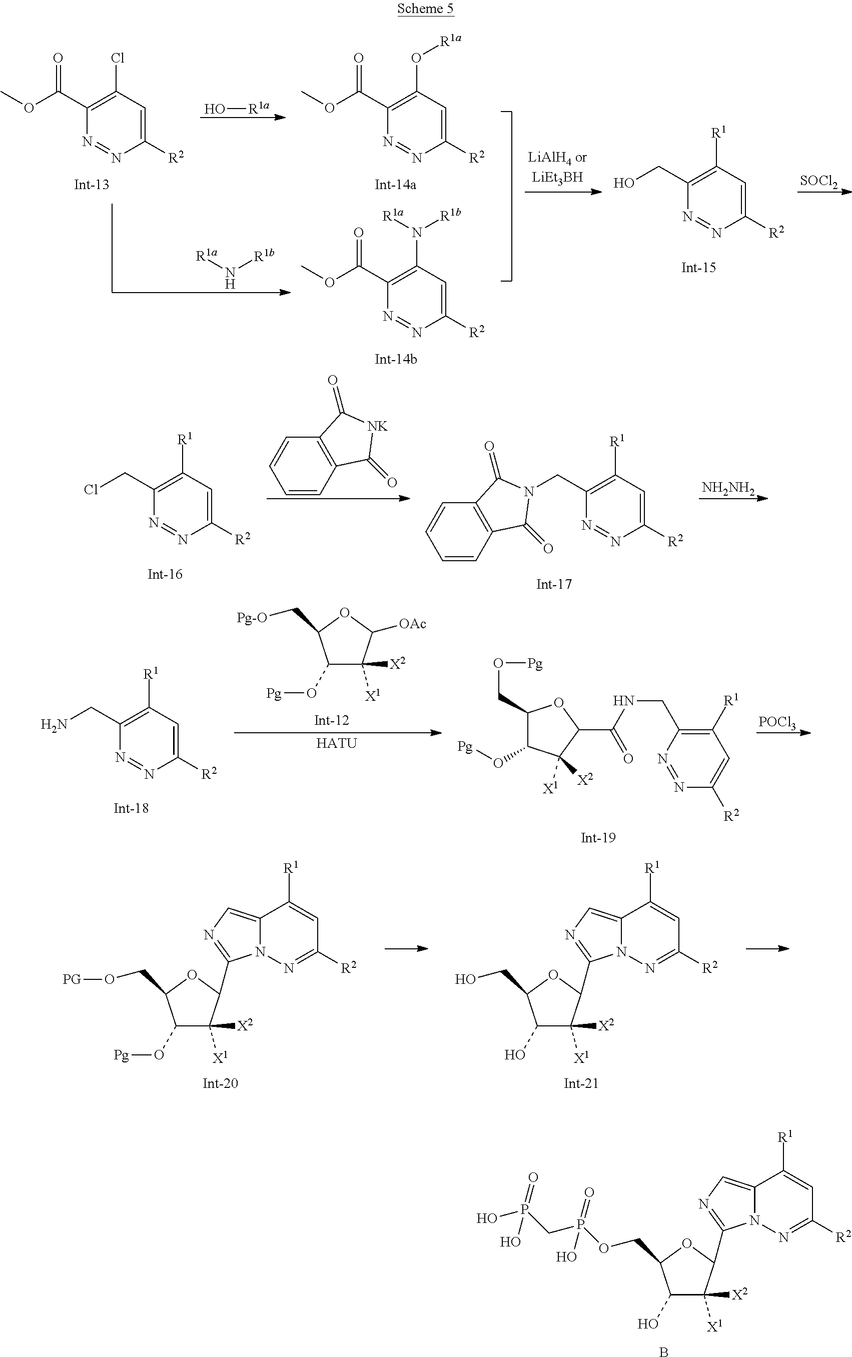

As shown in Scheme 5, Int-13 can be reacted with an alcohol in the presence of a base, such as sodium hydride in a solvent, such as THF, to give Int-14a. Alternatively, Int-13 can be reacted with a primary or secondary amine in the presence of a base, such as, for example, Et₃N or DIEA in a solvent, such as THF or EtOH to give Int-14b. Int-14a and Int-14b can be converted to the alcohol Int-15 with a reducing agent, such as lithium aluminum hydride (LiAlH₄), or lithium triethylborohydride (LiEt₃BH). Int-15 is converted to the chloride Int-16 by reacting with SOCl₂, PCl₅ or POCl₃. Reaction of Int-16 with potassium phthalimide in a suitable solvent, such as DMF, followed by reaction with hydrazine in a solvent such as water, ethanol or isopropanol gives the amine Int-18. Int-18 can be reacted with the acid Int-12 to yield the amide Int-19. Reaction conditions that are suitable for such amide formation are well known to those skilled in the art. For example HATU and DIEA or N,N′-dicyclohexylcarbodiimide (DCC) are suitable reagents for the formation of amides. Int-19 is cyclized to give Int-20 in the presence of POCl₃ in a solvent such as DCE. Int-20 can be deprotected to give Int-21. Deprotection will be accomplished by methods known to the skilled practitioner and are also described in “Greene's Protective Groups in Organic Synthesis”, John Wiley & Sons, Inc., 2014. For example, if the protection group is a benzoyl group (PG=Bz), hydrolysis with a base, such as K₂CO₃ will achieve the deprotection. If the protection group is a silyl ether, the deprotection can be accomplished, for example, by using Bu₄NF in THF. Many other protecting groups and methods for removing them are known to those skilled in the art. The ((hydroxy-phosphoryl)methyl) phosphonic acid B can be obtained from Int-21 by methods similar to the ones shown in Scheme 3 and described above.

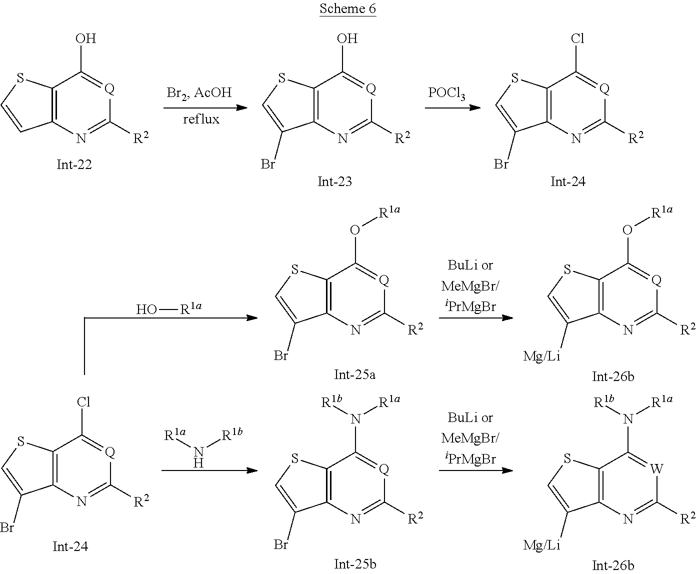

Yet, other compounds of this invention can be prepared as shown in Schemes 6 and 7. As shown in Scheme 6, Int-22 can be brominated by using bromine in acetic acid. The resulting Int-23 is converted to the chloride Int-24 by reacting with SOCl₂, PCl₅ or POCl₃. As shown in Scheme 6, Int-24 can be reacted with an alcohol in the presence of a base, such as sodium hydride in a solvent, such as THF, to give Int-25a. Alternatively, Int-24 can be reacted with a primary or secondary amine in the presence of a base, such as, for example, Et₃N or DIEA in a solvent, such as THF or EtOH to give Int-25b. Int-25a and Int-25b can be treated with an organometallic compound to give the metalated species Int-26a or Int-26b, respectively. This halogen-metal exchange can be accomplished, for example, with n-BuLi, sec-BuLi or tert-Buli or with MeMgBr and iPrMgBr in a solvent such as diethylether, dimethoxyethane, or THF.

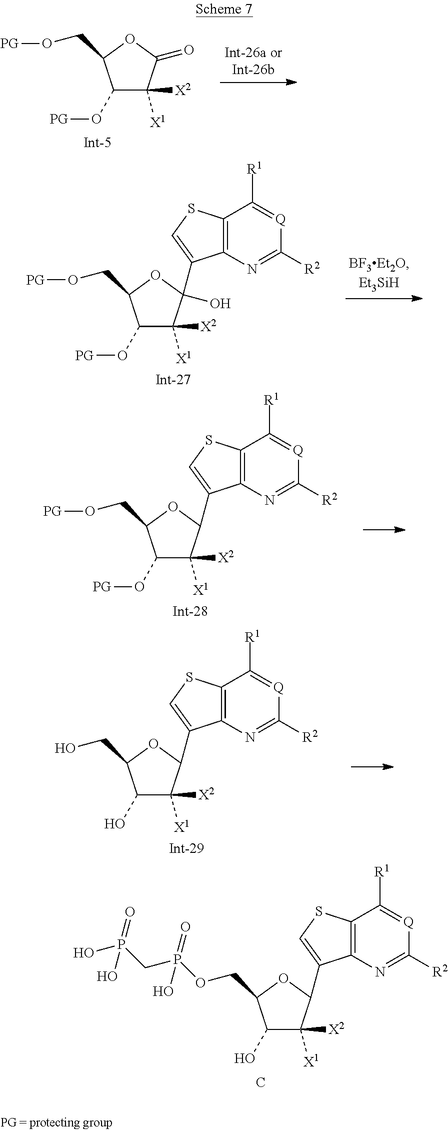

As shown in Scheme 7 Int-26a or Int-26b can be reacted with Int-5 to give compound Int-27. Int-27 can be converted to Int-28 with a silane in presence of a Lewis acid. For example, Et₃SiH in presence of BF₃.OEt₂ will accomplish this reaction. Int-28 can be deprotected to give Int-29. Deprotection will be accomplished by methods known to the skilled practitioner and are also described in “Greene's Protective Groups in Organic Synthesis”, John Wiley & Sons, Inc., 2014. For example, if the protection group is a benzyl ether (PG=Bn), hydrogen in presence of a catalyst, such as Pd on carbon, or BCl₃ in DCM will achieve the deprotection. If the protection group is a silyl ether, the deprotection can be accomplished, for example, by using Bu₄NF in THF. Many other protecting groups and methods for removing them are known to those skilled in the art. The ((hydroxy-phosphoryl)methyl) phosphonic acid C can be obtained from Int-29 by methods similar to the ones shown in Scheme 3 and described above.

### EXAMPLES

It is understood that the present disclosure has been made only by way of example, and that numerous changes in the combination and arrangement of parts can be resorted to by those skilled in the art without departing from the spirit and scope of the present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

The following abbreviations may be used herein:
˜ about
+ve or pos. ion positive ion
Δ heat
Ac Acetyl
ACN Acetonitrile
Ac₂O Acetic anhydride
AcOH Acetic acid
AMP Adenosine monophosphate
anh. anhydrous
aq aqueous
Bn benzyl
Boc tert-butyloxycarbonyl
Bz benzoyl
Calcd or Calc'd calculated
Conc. concentrated
D day(s) or doublet (NMR)
DCC N,N′-dicyclohexylcarbodiimide
DCE Dichloroethane
DCI 4,5-Dicyanoimidazole
DCM Dichloromethane
dd Doublet of doublets (NMR)
DEA Diethylamine
DIEA or DIPEA Diisopropylethylamine
DME 1,2-Dimethoxyethane
DMF N,N-Dimethylformamide
DMSO Dimethyl sulfoxide
EA or EtOAc Ethyl acetate
eq equivalent(s)
ESI Electrospray ionization
Et ethyl
Et₂O Diethyl ether
Et₃N Triethylamine
EtOH Ethyl alcohol
FA Formic acid
g gram(s)
h hour(s)
HATU O-(7-Azabenzotriazol-1-yl)-N,N,N′,N′-tetramethyluronium hexafluorophosphate, CAS #148893-10-1
Hex Hexanes
HMPA Hexamethylphosphoramide
HPLC High performance liquid chromatography
Hz Hertz
IPA or iPrOH Isopropyl alcohol
J Coupling constant (NMR) in Hz
KOAc Potassium acetate
LCMS, LC-MS or LC/MS Liquid chromatography-Mass spectrometry
LDA Lithium diisopropylamide
LHMDS or LiHMDS Lithium hexamethyldisilazide
m Multiplet (NMR)
M molar (mol L⁻¹)
Me methyl
MeCN Acetonitrile
MeI Iodomethane
MeOH Methyl alcohol
mg milligram(s)
min minute(s)
mL milliliter(s)
M mole(s)
MS Mass spectrometry
MsCl Methanesulfonyl chloride
MTBE or MtBE Methyl tert-butyl ether
m/z mass-to-charge ratio
NaHMDS Sodium hexamethyldisilazide
NaOtBu Sodium tert-butoxide
nBuLi n-Butyl lithium
NBS N-Bromo succinimide
nm Nanometer (wavelength)
NMR Nuclear magnetic resonance
P1 Product one; faster eluting isomer
P2 Product two; slower eluting isomer
PCC Pyridinium chlorochromate, CAS Number: 26299-14-9
PE Petroleum ether, CAS Number: 101316-46-5
PBS Phosphate buffered saline
PMB para-methoxybenzyl, 4-methoxybenzyl
Pr propyl
ppm parts per million
p-tol para-toluoyl
rac racemic
RT or rt or r.t. Room temperature
s Singlet (NMR)
sat. or sat'd or satd saturated
SFC Supercritical fluid chromatography
t Triplet (NMR)
TBSCl tert-Butyldimethylsilyl chloride
tBuOH tert-Butyl alcohol
TEA Triethylamine
TEAC Triethylammonium chloride
tert or t tertiary
TFA Triflouroacetic acid
THF Tetrahydrofuran
TLC Thin layer chromatography
TMS trimethylsilyl or trimethylsilane
Tris Tris(hydroxymethyl)aminomethane
v/v volume per volume

### Example S1

### Synthesis of (((((2R,3S,4R,5S)-5-(6-chloro-8-(cyclopentylamino)imidazo[1,2-b]pyridazin-3-yl)-3,4-dihydroxytetrahydrofuran-2-yl)methoxy)(hydroxy)phosphoryl)methyl)phosphonic acid (Compound No. 1)

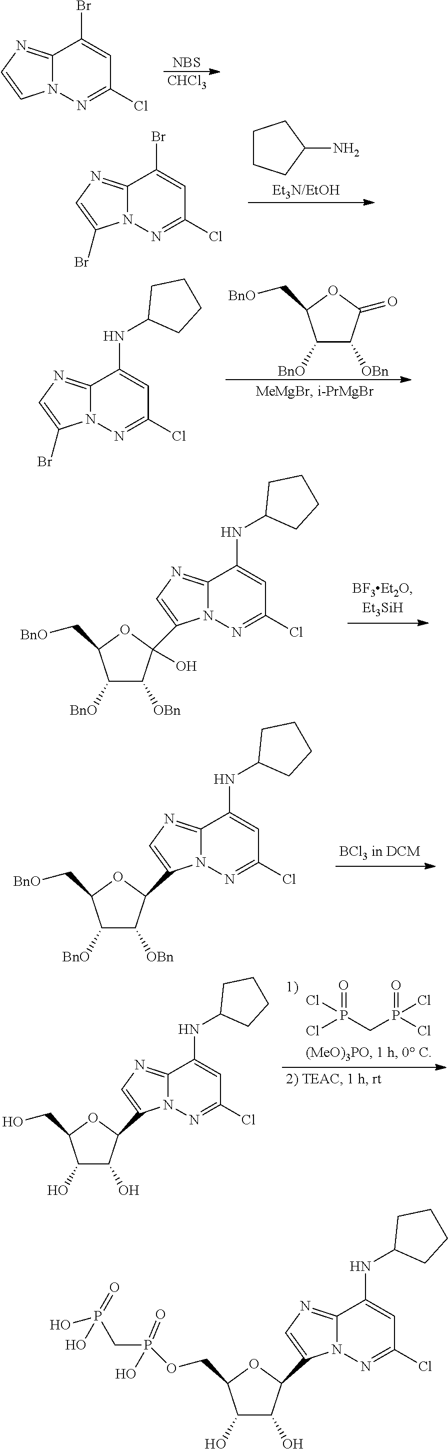

Step A: To a solution of 8-bromo-6-chloroimidazo[1,2-b]pyridazine (3.2 g, 13.77 mmol) in chloroform (35 mL) was added NBS (3.67 g, 20.66 mmol). Then the mixture was stirred at 80° C. for 1 h. The resulting solution was concentrated and purified by flash chromatography on silica gel (40 g, PE/EA=0-8%) to give 3,8-dibromo-6-chloroimidazo[1,2-b]pyridazine (2.3 g, 51%) as an off-white solid. Mass Spectrum (ESI) m/z=311.8 (M+1).

Step B: To a solution of 3,8-dibromo-6-chloroimidazo[1,2-b]pyridazine (2.3 g, 7.42 mmol) in EtOH (25 mL) was added triethylamine (1.58 g, 15.58 mmol) and cyclopentanamine (695 mg, 8.16 mmol). Then the mixture was stirred at 80° C. for 12 h. The resulting reaction was concentrated and purified by flash chromatography on silica gel (24 g, PE/EA=0-10%) to give 3-bromo-6-chloro-N-cyclopentylimidazo[1,2-b]pyridazin-8-amine (2.1 g, 90%) as an off-white solid. Mass Spectrum (ESI) m/z=316.5 (M+1).

Step C: To a solution of 3-bromo-6-chloro-N-cyclopentylimidazo[1,2-b]pyridazin-8-amine (1.5 g, 4.75 mmol) in THF (35 mL) was added bromo(methyl)magnesium (4.75 mL, 4.75 mmol), followed by chloro(isopropyl)magnesium (5.48 mL, 7.12 mmol). Then a solution of (3R,4R,5R)-3,4-bis(benzyloxy)-5-[(benzyloxy)methyl]oxolan-2-one (2.39 g, 5.7 mmol) in THF (15 mL) was added slowly. The mixture was stirred at 0° C. for 2 h, then it was quenched with sat. aq. NH₄Cl solution (50 mL) and extracted with EtOAc (50 mL×2). The combined organic layer was washed with brine, dried, concentrated and purified by flash chromatography on silica gel to give (3R,4R,5R)-3,4-bis(benzyloxy)-5-((benzyloxy)methyl)-2-(6-chloro-8-(cyclopentylamino) imidazo[1,2-b]pyridazin-3-yl)tetrahydrofuran-2-ol (1 g, 51% yield) as a yellow oil. Mass Spectrum (ESI) m/z=655.1 (M+1).

Step D: To a solution of (3R,4R,5R)-3,4-bis(benzyloxy)-5-[(benzyloxy)methyl]-2-[6-chloro-8-(cyclopentylamino)imidazo[1,2-b]pyridazin-3-yl]oxolan-2-ol (1 g, 1.53 mmol) in DCM (10 mL) was added triethylsilane (1.77 g, 15.26 mmol) and BF₃.OEt₂ (4.61 g, 15.26 mmol) at −78° C. Then the mixture was stirred from −78° C. to rt overnight. Saturated aqueous NaHCO₃ solution was added slowly, and the mixture was extracted with DCM (20 mL×2). The combined organic layer was washed with brine, dried, concentrated and purified by column chromatography on silica gel to give 3-((2S,3S,4R,5R)-3,4-bis(benzyloxy)-5-((benzyloxy)methyl)tetrahydrofuran-2-yl)-6-chloro-N-cyclopentylimidazo[1,2-b]pyridazin-8-amine (600 mg, 55% yield) as a colorless oil. Mass Spectrum (ESI) m/z=638.8 (M+1).

Step E: To a solution of 3-[(2S,3S,4R,5R)-3,4-bis(benzyloxy)-5-[(benzyloxy)methyl]oxolan-2-yl]-6-chloro-N-cyclopentylimidazo[1,2-b]pyridazin-8-amine (600 mg, 0.939 mmol) in DCM (10 mL) was added a solution of trichloroborane in DCM (1M, 9.4 mL) at −70° C., and the mixture was stirred at −70° C. for 3 h. Then a mixture of DCM and MeOH (1:1, 5 mL) was added slowly and the pH was adjusted to 7-8 with 7N NH₃ in MeOH. The mixture was concentrated and purified by flash chromatography on silica gel (5% NH₃ in MeOH/DCM=0-18%) to give (2S,3R,4S,5R)-2-(6-chloro-8-(cyclopentylamino)imidazo[1,2-b]pyridazin-3-yl)-5-(hydroxymethyl)tetrahydrofuran-3,4-diol (200 mg, 58% yield) as an off-white solid. Mass Spectrum (ESI) m/z=369.1 (M+1).

Step F: To a solution of (3R,4S,5R)-2-[6-chloro-8-(cyclopentylamino)imidazo[1,2-b]pyridazin-3-yl]-5-(hydroxymethyl)oxolane-3,4-diol (100 mg, 0.27 mmol) in trimethylphosphate (0.8 mL) at 0° C. was added a cold solution of methylenebis(phosphonic dichloride) (337 mg, 1.35 mmol) in trimethylphosphate (0.6 mL) dropwise. Then the reaction solution was stirred at 0° C. for 5 h. TEAC (0.5 M, 1.8 mL) was added to the reaction carefully and the reaction was stirred at this temperature for 15 mins, then warmed to room temperature and continued to stir for 1 h. Trimethylphosphate was extracted using tert-butyl methyl ether (5 mL×4) and the aqueous layer was basified with ammonium hydroxide to pH˜7-8. The aq. solution was purified by reverse phase preparative HPLC (Daisogel™—C18, 10 um, 250×50 mm, 0.2% TEAC in water/MeCN=90%-60%) to give (((((2R,3S,4R,5S)-5-(6-chloro-8-(cyclopentylamino)imidazo[1,2-b]pyridazin-3-yl)-3,4-dihydroxytetrahydrofuran-2-yl)methoxy)(hydroxy)phosphoryl)methyl)phosphonic acid (28 mg, 18.5% yield) as a white solid. ¹H NMR (400 MHz, D₂O) δ 7.61 (s, 1H), 6.22 (s, 1H), 5.16 (d, J=6.8 Hz, 11H), 4.62-4.51 (m, 1H), 4.32 (t, J=4.5 Hz, 11H), 4.17-4.14 (m, 1H), 3.98-3.91 (m, 2H), 3.87-3.80 (m, 1H), 2.08-1.97 (m, 4H), 1.70-1.50 (m, 6H). Mass Spectrum (ESI) m/z=525.0 (M−1).

### Example S2

### Synthesis of [({[(2R,3S,4R,5S)-5-(6-chloro-8-{[(1S)-1-(2-fluorophenyl)ethyl]amino}imidazo[1,2-b]pyridazin-3-yl)-3,4-dihydroxyoxolan-2-yl]methoxy}(hydroxy)phosphoryl)methyl]phosphonic acid (Compound No. 18)

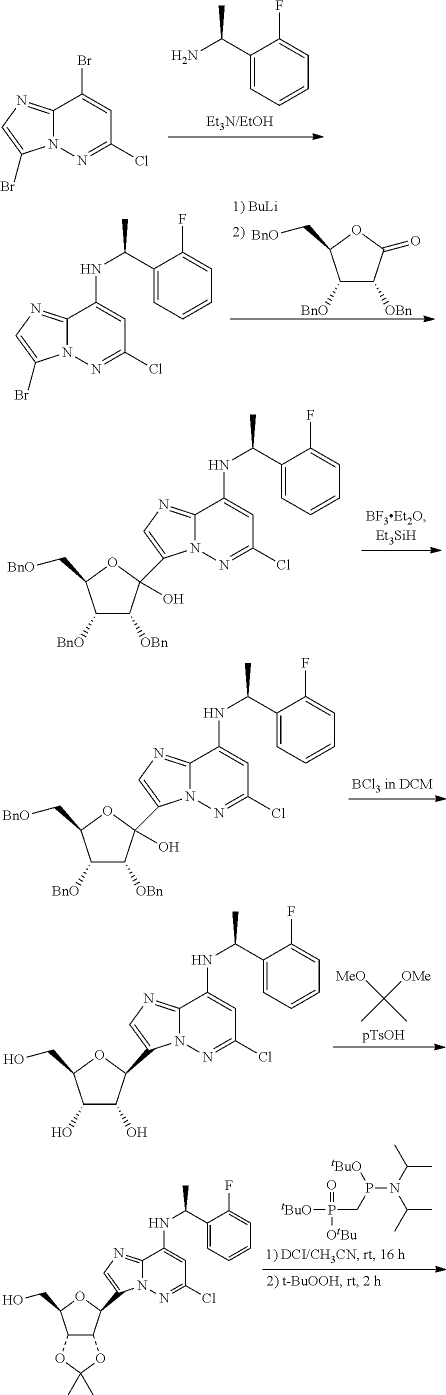

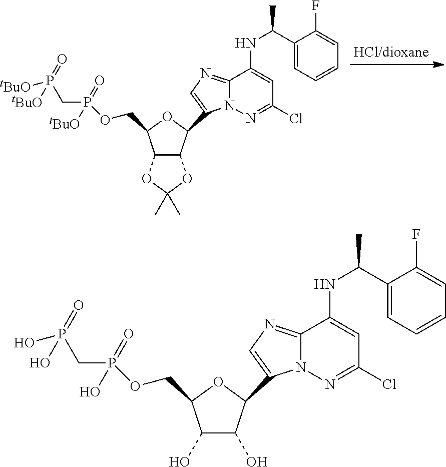

Step A: To a solution of 3,8-dibromo-6-chloroimidazo[1,2-b]pyridazine (16.7 g, 53.64 mmol) in EtOH (160 mL) was added trimethylamine (11.4 g, 107.28 mmol) and (1S)-1-(2-fluorophenyl)ethanamine (8.96 g, 64.36 mmol), then the mixture was stirred at 80° C. overnight. The resulting reaction was concentrated and purified by flash chromatography on silica gel (PE/EA=0-20%) to afford 3-bromo-6-chloro-N-[(1S)-1-(2-fluorophenyl)ethyl]imidazo[1,2-b]pyridazin-8-amine (19.5 g, 98% yield) as a yellow oil. Mass Spectrum (ESI) m/z=368.6 (M+1).

Step B: To a solution of 3-bromo-6-chloro-N-[(1S)-1-(2-fluorophenyl)ethyl]imidazo[1,2-b]pyridazin-8-amine (13 g, 35.17 mmol) in THF (100 mL) was added nBuLi (2.4M, 33.7 mL, 80.89 mmol) dropwise at −78° C. under nitrogen. The solution was stirred at this temperature for 0.5 h. Then a solution of (3R,4R,5R)-3,4-bis(benzyloxy)-5-[(benzyloxy)methyl]oxolan-2-one (16.2 g, 38.68 mmol) in THF (30 mL) was added. The reaction mixture was stirred at −78° C. for 1 h. Sat. aqueous NH₄Cl solution (130 mL) was added to quench the reaction carefully, and the mixture was extracted with EtOAc. The combined organic layers were dried over Na₂SO₄, filtered and concentrated to give the crude product. The residue was purified by flash chromatography on silica gel (PE/EA=4:1 to 1:1) to afford (3R,4R,5R)-3,4-bis(benzyloxy)-5-[(benzyloxy)methyl]-2-(6-chloro-8-{[(1S)-1-(2-fluorophenyl)ethyl]amino}imidazo[1,2-b]pyridazin-3-yl)oxolan-2-ol (16.5 g, 66% yield) as a yellow oil. Mass Spectrum (ESI) m/z=708.6 (M+1).

Step C: To a solution of (3R,4R,5R)-3,4-bis(benzyloxy)-5-[(benzyloxy)methyl]-2-(6-chloro-8-{[(1S)-1-(2-fluorophenyl)ethyl]amino}imidazo[1,2-b]pyridazin-3-yl)oxolan-2-ol (16.5 g, 23.3 mmol) in DCM (170 mL) under an atmosphere of N₂ was added BF₃.Et₂O (13.2 g, 93.06 mmol) and Et₃SiH (10.8 g, 93.06 mmol) at −78° C. successively. The resulting solution was stirred at 25° C. for 2 h. The reaction was quenched with sat. aq. NaHCO₃ solution and extracted with DCM. The organic layer was concentrated and purified by chromatography on silica gel (PE/EA=5:1 to 1:1) to afford 3-[(2S,3S,4R,5R)-3,4-bis(benzyloxy)-5-[(benzyloxy)methyl]oxolan-2-yl]-6-chloro-N-[(1S)-1-(2-fluorophenyl)ethyl]imidazo[1,2-b]pyridazin-8-amine (15.4 g, 95.7% yield) as a light yellow oil. Mass Spectrum (ESI) m/z=692.5 (M+1).

Step D: To a solution of 3-[(2S,3S,4R,5R)-3,4-bis(benzyloxy)-5-[(benzyloxy)methyl]oxolan-2-yl]-6-chloro-N-[(1S)-1-(2-fluorophenyl)ethyl]imidazo[1,2-b]pyridazin-8-amine (5.8 g, 8.37 mmol) in DCM (60 mL) was added a solution of BCl₃ in DCM (83.7 ml, 83.7 mmol) dropwise at −78° C. under a nitrogen atmosphere. The mixture was stirred at the same temperature for 1 h. Then the reaction was quenched with methanol/chloroform (1:1, 50 mL). After the reaction mixture had warmed to rt, it was neutralized with NH₃ in methanol (10%, 100 mL) and concentrated to give the crude product, which was purified by column chromatography on silica gel (DCM/MeOH=50:1 to 5:1) to give (2S,3R,4S,5R)-2-(6-chloro-8-{[(1S)-1-(2-fluorophenyl)ethyl]amino}imidazo[1,2-b]pyridazin-3-yl)-5-(hydroxymethyl)oxolane-3,4-diol (3.3 g, 93.3% yield) as a white solid. Mass Spectrum (ESI) m/z=422.6 (M+1).

Step E: (2S,3R,4S,5R)-2-(6-chloro-8-{[(1S)-1-(2-fluorophenyl)ethyl]amino}imidazo[1,2-b]pyridazin-3-yl)-5-(hydroxymethyl)oxolane-3,4-diol (3 g, 7.09 mmol) was dissolved in acetone (60 mL). 2,2-dimethoxypropane (15 mL) and p-TsOH.H₂O (1.53 g, 8.86 mmol) was added. The reaction mixture was stirred at rt overnight. Then it was diluted with EtOAc and carefully quenched with saturated aq. NaHCO₃ solution (20 mL) and extracted with EtOAc (3×20 mL). The combined organic layers were dried over MgSO₄, filtered, concentrated, and purified by chromatography on silica gel (PE/EA=4:1 to 1:1) to afford [(3aR,4R,6S,6aS)-6-(6-chloro-8-{[(1S)-1-(2-fluorophenyl)ethyl]amino}imidazo[1,2-b]pyridazin-3-yl)-2,2-dimethyl-tetrahydrofuro[3,4-d][1,3]dioxol-4-yl]methanol (2.2 g, 67% yield) as a light yellow solid. Mass Spectrum (ESI) m/z=462.7 (M+1).

Step F: To a solution of [(3aR,4R,6S,6aS)-6-(6-chloro-8-{[(1S)-1-(2-fluorophenyl)ethyl]amino} imidazo[1,2-b]pyridazin-3-yl)-2,2-dimethyl-tetrahydrofuro[3,4-d][1,3]dioxol-4-yl]methanol (1.66 g, 3.59 mmol) in MeCN (17 mL) was added di-tert-butyl {[(tert-butoxy)(diisopropylamino) phosphanyl]methyl}phosphonate (2.95 g, 7.18 mmol) and DCI (848 mg, 7.18 mmol). After the mixture was stirred at rt overnight, t-BuOOH (4.62 g, 35.9 mmol) was added and the mixture was stirred for another 1 h. Saturated aq. Na₂CO₃ solution was added and the mixture was extracted with DCM. The combined organic layers were dried over Na₂SO₄, filtered, concentrated and purified by chromatography on silica gel (DCM/MeOH=50:1 to 5:1) to give [(3aR,4R,6S,6aS)-6-(6-chloro-8-{[(1S)-1-(2-fluorophenyl)ethyl]amino}imidazo[1,2-b]pyridazin-3-yl)-2,2-dimethyl-tetrahydrofuro[3,4-d][1,3]dioxol-4-yl]methyl tert-butyl {[bis(tert-butoxy)phosphoryl]methyl}phosphonate (2 g, 70.7% yield). Mass Spectrum (ESI) m/z=789.5 (M+1).

Step G: To a mixture of [(3aR,4R,6S,6aS)-6-(6-chloro-8-{[(1S)-1-(2-fluorophenyl)ethyl]amino} imidazo[1,2-b]pyridazin-3-yl)-2,2-dimethyl-tetrahydrofuro[3,4-d][1,3]dioxol-4-yl]methyl tert-butyl {[bis(tert-butoxy)phosphoryl]methyl}phosphonate (1.6 g, 2.03 mmol) in dioxane (25 mL) was added a solution of HCl in dioxane (5 mL) and ethylene glycol (630 mg, 10.15 mmol), carefully. The reaction was stirred at 25° C. for 2 h. Then the reaction was concentrated and purified by reverse phase preparative HPLC (Daisogel™—C18, 10 um, 250×50 mm, gradient of 0.5% aq. HCOOH/ACN from 75:25 to 55:45). Product containing fractions were pooled and lyophilized to give the final product [({[(2R,3S,4R,5S)-5-(6-chloro-8-{[(1S)-1-(2-fluorophenyl)ethyl]amino}imidazo[1,2-b]pyridazin-3-yl)-3,4-dihydroxyoxolan-2-yl]methoxy}(hydroxy)phosphoryl)methyl]phosphonic acid (497.5 mg, 42.2% yield) as a white solid. ¹H NMR (400 MHz, D₂O) δ 8.09 (s, 1H), 7.40-7.25 (m, 2H), 7.19-7.07 (m, 2H), 6.41 (s, 1H), 5.29 (d, J=5.1 Hz, 1H), 5.10-5.02 (m, 1H), 4.56-4.50 (m, 1H), 4.38-4.30 (m, 1H), 4.23-4.17 (m, 1H), 4.12-4.00 (m, 2H), 2.18 (t, J=19.8 Hz, 2H), 1.66-1.55 (m, 3H). Mass Spectrum (ESI) m/z=580.8 (M+1).

### Example S3

### Synthesis of (((((2R,3S,4R,5S)-5-(6-chloro-8-((S)-2-phenylpyrrolidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 25)

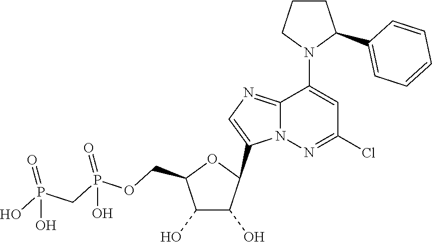 [SMILES: O=P(O)(O)CP(=O)(O)OC[C@H]1O[C@@H](c2cnc3c(N4CCC[C@H]4c4ccccc4)cc(Cl)nn23)[C@H](O)[C@@H]1O]

(((((2R,3S,4R,5S)-5-(6-chloro-8-((S)-2-phenylpyrrolidin-1-yl)imidazo[1,2-b]pyridazin-3-yl)-3,4-dihydroxytetrahydrofuran-2-yl)methoxy)(hydroxy)phosphoryl)methyl)phosphonic acid was synthesized by procedures similar to the ones described in Example S2, replacing (S)-1-(2-fluorophenyl)ethan-1-amine in Step A with (S)-2-phenylpyrrolidine hydrochloride.

¹H NMR (400 MHz, D₂O) δ 7.59 (s, 1H), 7.29-7.21 (m, 6H), 5.77 (s, 1H), 5.13-5.12 (d, J=6.5 Hz, 1H), 4.54-4.52 (m, 1H), 4.34-4.31 (m, 1H), 4.12 (d, J=4.4 Hz, 1H), 4.06-3.87 (m, 3H), 2.93-2.90 (m, 1H), 2.40-2.38 (m, 1H), 1.95-1.92 (m, 5H). Mass Spectrum (ESI) m/z=587.0 (M−1).

### Example S4

### Synthesis of (((((2R,3S,4R,5S)-5-(6-chloro-8-(hexahydrocyclopenta[c]pyrrol-2 (1H)-yl)imidazo[1,2-b]pyridazin-3-yl)-3,4-dihydroxytetrahydrofuran-2-yl)methoxy)(hydroxy)phosphoryl)methyl) phosphonic acid (Compound No. 30)

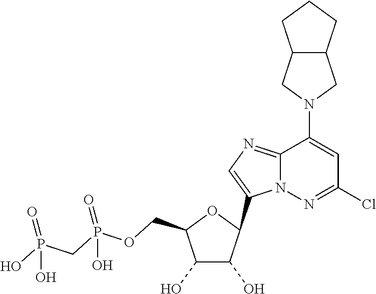 [SMILES: O=P(O)(O)CP(=O)(O)OC[C@H]1O[C@@H](c2cnc3c(N4CC5CCCC5C4)cc(Cl)nn23)[C@H](O)[C@@H]1O]

(((((2R,3S,4R,5S)-5-(6-chloro-8-(hexahydrocyclopenta[c]pyrrol-2 (1H)-yl)imidazo[1,2-b]pyridazin-3-yl)-3,4-dihydroxytetrahydrofuran-2-yl)methoxy)(hydroxy)phosphoryl)methyl) phosphonic acid was synthesized by procedures similar to the ones described in Example S2, replacing (S)-1-(2-fluorophenyl)ethan-1-amine in Step A with octahydrocyclopenta[c]pyrrole.

¹H NMR (400 MHz, D₂O) δ 8.04 (s, 1H), 6.43 (s, 1H), 5.28 (d, J=4.6 Hz, 1H), 4.57-4.46 (m, 1H), 4.38-4.29 (m, 1H), 4.20-4.14 (m, 1H), 4.13-4.00 (m, 2H), 3.95-3.84 (m, 2H), 3.60-3.48 (m, 2H), 2.86-2.78 (m, 2H), 2.18 (t, J=18.6 Hz, 2H), 1.88-1.79 (m, 2H), 1.75-1.68 (m, 1H), 1.64-1.56 (m, 1H), 1.50-1.42 (m, 2H). Mass Spectrum (ESI) m/z=550.7 (M−1).

### Example S5

### Synthesis of (((((2R,3S,4R,5S)-5-(6-chloro-8-(cyclohexylamino)imidazo[1,2-b]pyridazin-3-yl)-3,4-dihydroxytetrahydrofuran-2-yl)methoxy)(hydroxy)phosphoryl)methyl)phosphonic acid (Compound No. 11)

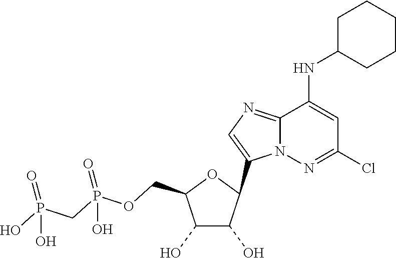 [SMILES: O=P(O)(O)CP(=O)(O)OC[C@H]1O[C@@H](c2cnc3c(NC4CCCCC4)cc(Cl)nn23)[C@H](O)[C@@H]1O]

(((((2R,3S,4R,5S)-5-(6-chloro-8-(cyclohexylamino)imidazo[1,2-b]pyridazin-3-yl)-3,4-dihydroxytetrahydrofuran-2-yl)methoxy)(hydroxy)phosphoryl)methyl)phosphonic acid was synthesized by procedures similar to the ones described in Example S2, replacing (S)-1-(2-fluorophenyl)ethan-1-amine in Step A with 1-aminocyclohexane.

¹H NMR (400 MHz, D₂O) δ 8.01 (s, 1H), 6.69 (s, 1H), 5.25 (d, J=5.0 Hz, 1H), 4.51 (t, J=5.0 Hz, 1H), 4.31 (t, J=5.2 Hz, 1H), 4.20-4.14 (m, 1H), 4.11-3.97 (m, 2H), 3.54-3.46 (m, 1H), 2.19 (t, J=20.0 Hz, 2H), 2.00-1.90 (m, 2H), 1.74-1.64 (m, 2H), 1.58-1.50 (m, 1H), 1.39-1.25 (m, 4H), 1.24-1.11 (m, 1H). Mass Spectrum (ESI) m/z=540.6 (M+1).

### Example S6

### Synthesis of (((((2R,3S,4R,5S)-5-(8-(bicyclo[2.2.1]heptan-2-ylamino)-6-chloroimidazo[1,2-b]pyridazin-3-yl)-3,4-dihydroxytetrahydrofuran-2-yl)methoxy)(hydroxy)phosphoryl)methyl)phosphonic acid (Compound No. 12)

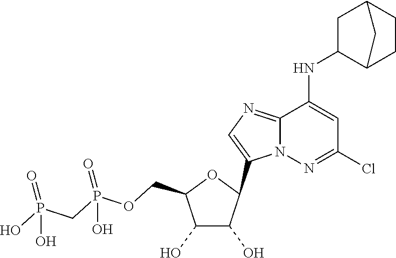 [SMILES: O=P(O)(O)CP(=O)(O)OC[C@H]1O[C@@H](c2cnc3c(NC4CC5CCC4C5)cc(Cl)nn23)[C@H](O)[C@@H]1O]

(((((2R,3S,4R,5S)-5-(8-(bicyclo[2.2.1]heptan-2-ylamino)-6-chloroimidazo[1,2-b]pyridazin-3-yl)-3,4-dihydroxytetrahydrofuran-2-yl)methoxy)(hydroxy)phosphoryl)methyl)phosphonic acid was synthesized by procedures similar to the ones described in Example S2, replacing (S)-1-(2-fluorophenyl)ethan-1-amine in Step A with bicyclo[2.2.1]heptan-2-amine.

¹H NMR (400 MHz, D₂O) δ 8.01 (s, 1H), 6.65 (d, J=2.3 Hz, 1H), 5.24 (t, J=4.5 Hz, 1H), 4.54-4.47 (m, 1H), 4.34-4.27 (m, 1H), 4.21-4.14 (m, 1H), 4.12-3.96 (m, 2H), 3.87-3.80 (m, 1H), 2.60-2.50 (m, 1H), 2.30-2.06 (m, 4H), 1.55-1.39 (m, 3H), 1.37-1.27 (m, 2H), 1.25-1.15 (m, 1H), 1.15-0.95 (m, 1H). Mass Spectrum (ESI) m/z=552.5 (M+1).

### Example S7

### Synthesis of (((((2R,3S,4R,5S)-5-(2-chloro-4-(cyclopentylamino)imidazo[1,5-b]pyridazin-7-yl)-3,4-dihydroxytetrahydrofuran-2-yl)methoxy)(hydroxy)phosphoryl)methyl)phosphonic acid (Compound No. 39)

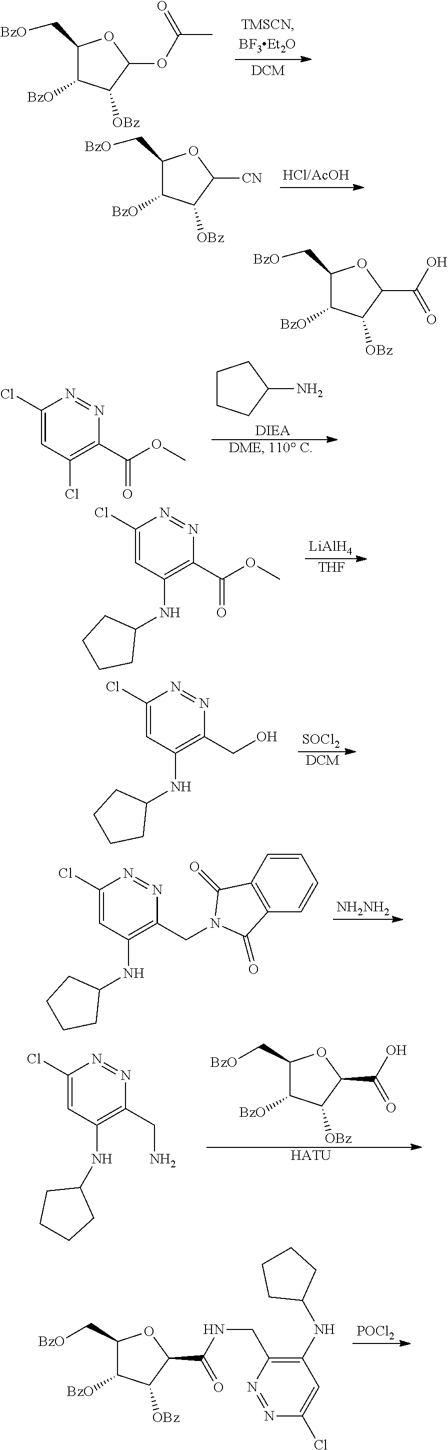

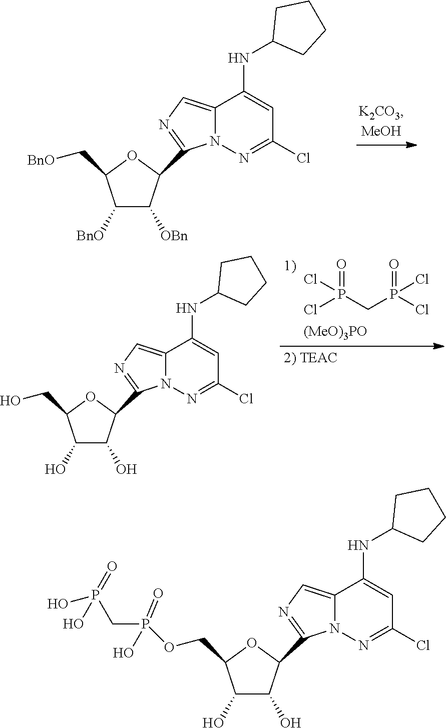 [SMILES: "(MeO)3PO" ; Clc1cc(NC2CCCC2)c2cnc([C@@H]3O[C@H](COCc4ccccc4)[C@@H](OCc4ccccc4)[C@H]3OCc3ccccc3)n2n1 ; OC[C@H]1O[C@@H](c2ncc3c(NC4CCCC4)cc(Cl)nn23)[C@H](O)[C@@H]1O ; O=P(Cl)(Cl)CP(=O)(Cl)Cl ; O=P(O)(O)CP(=O)(O)OC[C@H]1O[C@@H](c2ncc3c(NC4CCCC4)cc(Cl)nn23)[C@H](O)[C@@H]1O]

Step A: To a solution of methyl 4,6-dichloropyridazine-3-carboxylate (9 g, 43.48 mmol) in DME (90 mL) was added diethyl(isopropyl)amine (11.2 g, 86.95 mmol), followed by cyclopentanamine (3.7 g, 43.48 mmol). The mixture was stirred at 100° C. for 1 h. Water (100 mL) was added and the mixture was extracted with EA (100 mL×2) The combined organic layers were washed with brine, dried over MgSO₄, filtered, and the filtrate was concentrated. The residue purified by flash chromatography on silica gel (120 g, EA/PE=0-30%) to give methyl 6-chloro-4-(cyclopentylamino)pyridazine-3-carboxylate (6.7 g, 54% yield). Mass Spectrum (ESI) m/z=255.9 (M+1).

Step B: LiAlH₄ (1.2 g, 32.75 mmol) in THF (30 ML) was cooled to 0° C., then methyl 6-chloro-4-(cyclopentylamino)pyridazine-3-carboxylate (6.7 g, 26.2 mmol) in THF (30 mL) was added slowly. The mixture was stirred at 0° C. for 2 h. Water (1.2 mL) was added carefully, followed by 10% aq. NaOH (1.2 mL) and water (3.6 mL). The resulting mixture was filtered and the filtrate was concentrated and purified by flash chromatography on silica gel (40 g, EA/PE=0-60%) to give (6-chloro-4-(cyclopentylamino)pyridazin-3-yl)methanol (4 g, 64% yield) as a solid. Mass Spectrum (ESI) m/z=227.9 (M+1).

Step C: To a solution of [6-chloro-4-(cyclopentylamino)pyridazin-3-yl]methanol (4 g, 17.57 mmol) in DCM (40 mL) was added SOCl₂ (10 mL). The mixture was stirred at rt for 4 h. Then the reaction was concentrated, and the residue was re-dissolved in DMF (40 mL). Potassium phthalimide (CAS #1074-82-4, 3.3 g, 17.57 mmol) was added. The resulting mixture was stirred at rt overnight. Water (50 mL) was added, and the mixture was extracted with EA (50 mL×2). The combined organic layers were washed with brine, dried over MgSO₄, filtered, and the filtrate was concentrated. The residue was purified by flash chromatography on silica gel (40 g, EA/PE=0-58%) to give 2-((6-chloro-4-(cyclopentylamino)pyridazin-3-yl)methyl)isoindoline-1,3-dione (3.5 g, 53% yield) as a solid. Mass Spectrum (ESI) m/z=356.8 (M+1).

Step D: To a solution of 2-{[6-chloro-4-(cyclopentylamino)pyridazin-3-yl]methyl}isoindole-1,3-dione (3.5 g, 9.8 mmol) in EtOH (35 mL) was added N₂H₄ (5 mL). The mixture was stirred at 80° C. for 3 h, then filtered and concentrated to give 3-(aminomethyl)-6-chloro-N-cyclopentylpyridazin-4-amine (2.1 g, 89% yield) as an off-white solid. Mass Spectrum (ESI) m/z=226.9 (M+1).

Step E: To a mixture of (2S,3R,4R,5R)-2-acetoxy-5-((benzoyloxy)methyl)tetrahydrofuran-3,4-diyl dibenzoate (10 g, 19.82 mmol) and trimethylsilanecarbonitrile (2.9 g, 29.73 mmol) in DCM (60 mL) was added boron trifluoride etherate (3.4 g, 99.11 mmol) at 0° C. Then the mixture was warmed to rt and stirred for 2 h. Aqueous NaHCO₃ solution was added and the organic layer was dried over MgSO₄, filtered, and the filtrate was concentrated. The residue was purified by flash chromatography on silica gel (EA/PE=0-20%) to give (2R,3R,4S,5S)-2-((benzoyloxy)methyl)-5-cyanotetrahydrofuran-3,4-diyl dibenzoate (7 g, 67% yield) as a colorless oil. Mass Spectrum (ESI) m/z=493.7 (M+1).

Step F: To a solution of (2R,3R,4S,5S)-2-((benzoyloxy)methyl)-5-cyanotetrahydrofuran-3,4-diyl dibenzoate (7 g, 14.85 mmol) in AcOH (20 mL) was added conc. HCl (20 mL), then the mixture was stirred at 50° C. for 4 h. The reaction mixture was concentrated and the residue was purified by flash chromatography on silica gel (40 g, EA/0.1% AcOH in PE=0-70%) to give (2R,3R,4R,5R)-3,4-bis(benzoyloxy)-5-((benzoyloxy)methyl)tetrahydrofuran-2-carboxylic acid (5.6 g, 69% yield). Mass Spectrum (ESI) m/z=490.9 (M+1).

Step G: To a solution of 3-(aminomethyl)-6-chloro-N-cyclopentylpyridazin-4-amine (1 g, 4.41 mmol) and (2R,3R,4R,5R)-3,4-bis(benzoyloxy)-5-((benzoyloxy)methyl)tetrahydrofuran-2-carboxylic acid (2.16 g, 4.41 mmol) in DMF (15 mL) was added DIEA (1.14 g, 8.82 mmol) and HATU (1.76 g, 4.63 mmol). Then the mixture was stirred at rt for 2 h. Water (20 mL) was added and the mixture was extracted with EA (20 mL×2). The combined organic layers were washed with brine, dried over MgSO₄, filtered, and the filtrate was concentrated. The residue was purified by flash chromatography on silica gel (20 g, EA/PE=0-40%) to give (2R,3R,4R,5R)-2-((benzoyloxy)methyl)-5-(((6-chloro-4-(cyclopentylamino)pyridazin-3-yl)methyl)carbamoyl)tetrahydrofuran-3,4-diyl dibenzoate (1.4 g, 43% yield). Mass Spectrum (ESI) m/z=698.4 (M+1).

Step H: To a mixture of (2R,3R,4R,5R)-4-(benzoyloxy)-5-[(benzoyloxy)methyl]-2-({[6-chloro-4-(cyclopentylamino)pyridazin-3-yl]methyl}carbamoyl)oxolan-3-yl benzoate (1.4 g, 2 mmol) in 1,2-dichloroethane (10 mL) was added POCl₃ (3 g, 20 mmol). Then the mixture was stirred at 85° C. for 24 h. The reaction was concentrated and purified by flash chromatography on silica gel (12 g, EA/PE=0-20%) to give (2R,3R,4S,5S)-2-((benzoyloxy)methyl)-5-(2-chloro-4-(cyclopentylamino)imidazo[1,5-b]pyridazin-7-yl)tetrahydrofuran-3,4-diyl dibenzoate (740 mg, 51% yield) as a yellow oil. Mass Spectrum (ESI) m/z=680.5 (M+1).

Step I: To a solution of (2R,3R,4S,5S)-4-(benzoyloxy)-2-[(benzoyloxy)methyl]-5-[2-chloro-4-(cyclopentylamino)imidazo[1,5-b]pyridazin-7-yl]oxolan-3-yl benzoate (200 mg, 0.29 mmol) in MeOH (5 mL) was added K₂CO₃ (123 mg, 0.89 mmol). Then the mixture was stirred at rt for 3 h. The resulting mixture was concentrated and purified by flash chromatography on silica gel (4 g, MeOH/DCM=0-10%) to give (2S,3R,4S,5R)-2-(2-chloro-4-(cyclopentylamino)imidazo[1,5-b]pyridazin-7-yl)-5-(hydroxymethyl)tetrahydrofuran-3,4-diol (80 mg, 74% yield) as an off-white solid. Mass Spectrum (ESI) m/z=368.8 (M+1).

Step J: To a solution of (2S,3R,4S,5R)-2-[2-chloro-4-(cyclopentylamino)imidazo[1,5-b]pyridazin-7-yl]-5-(hydroxymethyl)oxolane-3,4-diol (80 mg, 0.22 mmol) in trimethylphosphate (1.2 mL) at 0° C. was added a cold solution of methylenebis(phosphonic dichloride) (270 mg, 1.08 mmol) in trimethylphosphate (0.8 mL) dropwise. Then the reaction solution was stirred at 0° C. for 3 h. TEAC (0.5 M, 1.5 mL) was added to the reaction carefully and the reaction was stirred at this temperature for 15 mins, then warmed to room temperature and continued to stir for 1 h. Trimethylphosphate was extracted using tert-butyl methyl ether (5 mL×4) and the aqueous layer was basified with ammonium hydroxide to pH˜7-8. The aq. solution was purified by reverse phase preparative HPLC (Daisogel™—C18, 10 um, 250×50 mm, 0.2% TEAC in water/MeCN=90%-60%) and ion exchange resin (Dowex 50WX8—100 cation exchange resin, eluting with pure water) to give (((((2R,3S,4R,5S)-5-(2-chloro-4-(cyclopentylamino)imidazo[1,5-b]pyridazin-7-yl)-3,4-dihydroxytetrahydrofuran-2-yl)methoxy)(hydroxy)phosphoryl)methyl)phosphonic acid (5 mg, 4.3% yield) as a white solid.

¹H NMR (400 MHz, D₂O) δ 7.52 (s, 1H), 5.88 (s, 1H), 5.32 (d, J=4 Hz, 1H), 4.79-4.74 (m, 1H), 4.38-4.33 (m, 1H), 4.21-4.15 (m, 1H), 3.98-3.86 (m, 3H), 2.09-1.95 (m, 4H), 1.68-1.50 (m, 6H). Mass Spectrum (ESI) m/z=524.7 (M−1).

### Example S8

### Synthesis of (((((2R,3S,4R,5S)-5-(2-chloro-4-((S)-2-phenylpyrrolidin-1-yl)imidazo[1,5-b]pyridazin-7-yl)-3,4-dihydroxytetrahydrofuran-2-yl)methoxy)(hydroxy)phosphoryl)methyl)phosphonic acid (Compound No. 58)

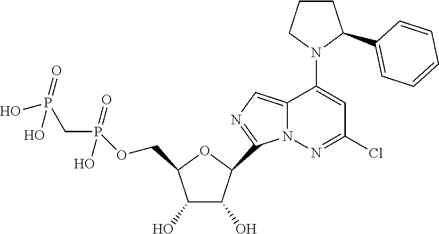 [SMILES: O=P(O)(O)CP(=O)(O)OC[C@H]1O[C@@H](c2ncc3c(N4CCC[C@H]4c4ccccc4)cc(Cl)nn23)[C@H](O)[C@@H]1O]

(((((2R,3S,4R,5S)-5-(2-chloro-4-((S)-2-phenylpyrrolidin-1-yl)imidazo[1,5-b]pyridazin-7-yl)-3,4-dihydroxytetrahydrofuran-2-yl)methoxy)(hydroxy)phosphoryl)methyl)phosphonic acid was synthesized by procedures similar to the ones described in Example S7, replacing cyclopentanamine in step A with (S)-2-phenylpyrrolidine.

¹H NMR (400 MHz, D₂O) δ 8.24 (s, 1H), 7.41-7.12 (m, 5H), 5.61-5.49 (m, 2H), 5.10-1.95 (m, 1H), 4.60-4.50 (m, 1H), 4.35-4.15 (m, 3H), 4.12-3.95 (m, 2H), 2.51-2.37 (m, 1H), 2.25-1.87 (m, 5H). Mass Spectrum (ESI) m/z=586.5 (M−1).

### Example S9

### Synthesis of [({[(2R,3S,4R,5S)-5-(2-chloro-4-{hexahydro-1H-cyclopenta[c]pyrrol-2-yl}imidazo[1,5-b]pyridazin-7-yl)-3,4-dihydroxyoxolan-2-yl]methoxy}(hydroxy)phosphoryl)methyl]phosphonic acid (Compound No. 62)

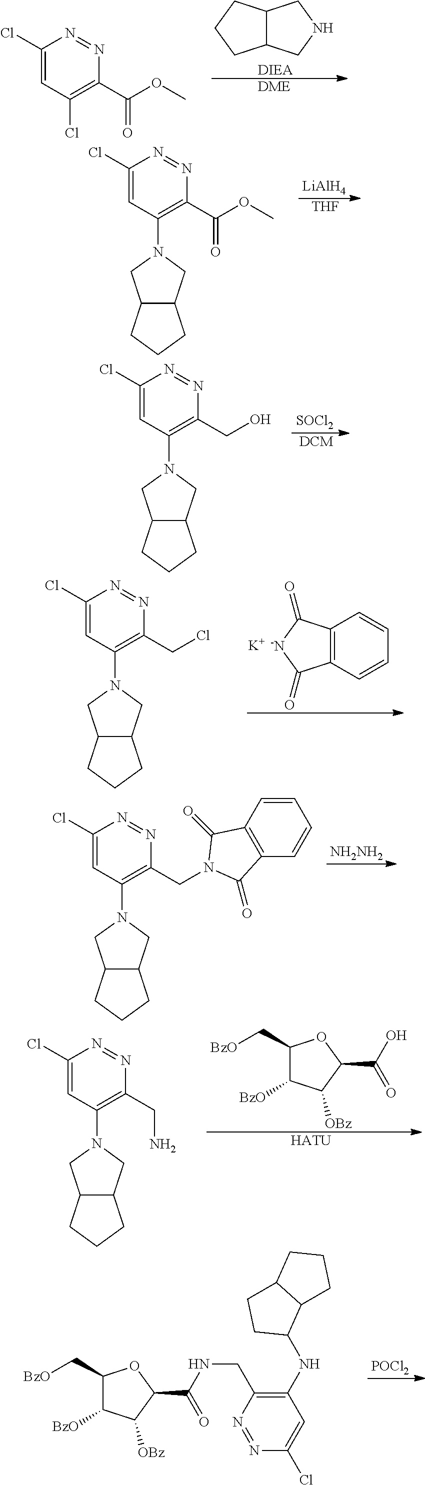

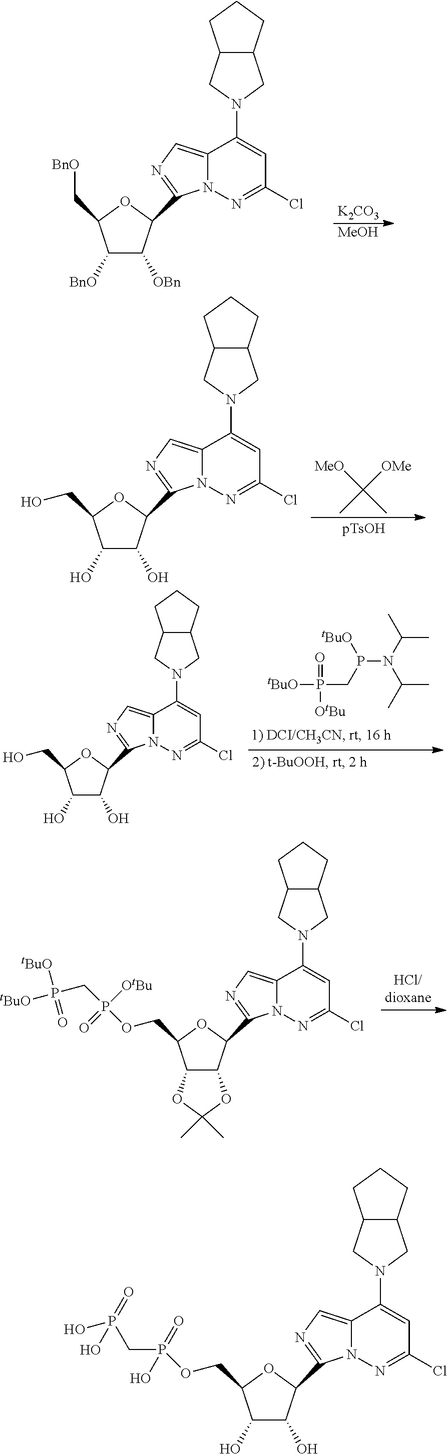

Step A: To a solution of methyl 4,6-dichloropyridazine-3-carboxylate (30 g, 0.144 mol) in DMA (150 mL) was added N,N-diisopropylethylamine (37 g, 0.289 mol) and octahydrocyclopenta[c]pyrrole (17.7 g, 0.159 mol). The reaction mixture was stirred at 70° C. for 30 min, then volatiles were removed by rotary evaporation. The crude product was purified by silica column chromatography (60 g, PE/EA=70:30) to give methyl 6-chloro-4-{hexahydro-1H-cyclopenta[c]pyrrol-2-yl}pyridazine-3-carboxylate (34 g, 74% yield) as a white solid. Mass Spectrum (ESI) m/z=281.9 (M+1).

Step B: To a solution of methyl 6-chloro-4-{hexahydro-1H-cyclopenta[c]pyrrol-2-yl}pyridazine-3-carboxylate (23 g, 0.081 mol) in THF (300 ML) was added DIBAL-H (163 mL) dropwise at 0° C. and the mixture was stirred at this temperature for 6 h. After the reaction was completed, water (80 mL) and 1N HCl (163 mL) was added to the solution at 0° C. Then saturated aqueous sodium bicarbonate solution was added to the mixture at room temperature. The mixture was extracted with DCM (400 mL). The organic layer was dried over anhydrous sodium sulfate, filtered, concentrated and the residue was purified by silica gel column chromatography to give (6-chloro-4-{hexahydro-1H-cyclopenta[c]pyrrol-2-yl}pyridazin-3-yl)methanol (20 g, 87% yield) as a solid. Mass Spectrum (ESI) m/z=253.9 (M+1).

Step C: To a solution of (6-chloro-4-{hexahydro-1H-cyclopenta[c]pyrrol-2-yl}pyridazin-3-yl)methanol (2 g, 7 mmol) in DCM (15 mL) was added SOCl₂ (4 mL). The reaction mixture was stirred at rt for 4 h. Then the solution was concentrated and purified by flash chromatography on silica gel to give 6-chloro-3-(chloromethyl)-4-{hexahydro-1H-cyclopenta[c]pyrrol-2-yl}pyridazine (1.6 g, 67% yield) as a solid. Mass Spectrum (ESI) m/z=271.9 (M+1).

Step D: To a mixture of 6-chloro-3-(chloromethyl)-4-{hexahydro-1H-cyclopenta[c]pyrrol-2-yl}pyridazine (1.6 g, 5 mmol) in DMF (10 mL) was added potassium phthalimide (1.1 g, 5 mmol). The mixture was stirred at rt for 16 h. Water (50 mL) was added, and the mixture was extracted with EA (50 mL×2). The combined organic layers were washed with brine, dried over MgSO₄, filtered, concentrated and purified by flash chromatography on silica gel (3 g, EA/PE=0-58%) to give 2-[(6-chloro-4-{hexahydro-1H-cyclopenta[c]pyrrol-2-yl}pyridazin-3-yl)methyl]isoindole-1,3-dione (2.45 g, 98% yield) as a solid. Mass Spectrum (ESI) m/z=382.8 (M+1).

Step E: To a solution of 2-[(6-chloro-4-{hexahydro-1H-cyclopenta[c]pyrrol-2-yl}pyridazin-3-yl)methyl]isoindole-1,3-dione (2.45 g, 6 mmol) in EtOH (15 mL) was added N₂H₄.H₂O (4 mL). The mixture was stirred at 80° C. for 3 h, filtered and the filtrate was concentrated to give (6-chloro-4-{hexahydro-1H-cyclopenta[c]pyrrol-2-yl}pyridazin-3-yl)methanamine (1.75 g, 94% yield) as a brown solid. Mass Spectrum (ESI) m/z=252.9 (M+1).

Step F: To a mixture of (6-chloro-4-{hexahydro-1H-cyclopenta[c]pyrrol-2-yl}pyridazin-3-yl)methanamine (900 mg, 3.56 mmol) and (2R,3R,4R,5R)-3,4-bis(benzoyloxy)-5-[(benzoyloxy)methyl]oxolane-2-carboxylic acid (1.7 g, 3.56 mmol) in DMF (20 mL) was added DIEA (920 mg, 7.12 mmol) and HATU (1.5 g, 3.92 mmol). Then the mixture was stirred at rt for 2 h. Water (50 mL) was added, the mixture was extracted with EA (50 mL×2), and the combined organic layers were washed with brine, dried over MgSO₄, concentrated and purified by flash chromatography on silica gel (EA/PE=0-40%) to give (2R,3R,4R,5R)-4-(benzoyloxy)-5-[(benzoyloxy)methyl]-2-{[(6-chloro-4-{hexahydro-1H-cyclopenta[c]pyrrol-2-yl}pyridazin-3-yl)methyl]carbamoyl}oxolan-3-yl benzoate (1.1 g, 38% yield) as a yellow oil. Mass Spectrum (ESI) m/z=724.5 (M+1).

Step G: To a mixture of (2R,3R,4R,5R)-4-(benzoyloxy)-5-[(benzoyloxy)methyl]-2-{[(6-chloro-4-{hexahydro-1H-cyclopenta[c]pyrrol-2-yl}pyridazin-3-yl)methyl]carbamoyl}oxolan-3-yl benzoate (670 mg, 0.92 mmol) in DCE (8 mL) was added phosphorus oxychloride (1.4 g, 9.2 mmol), then the mixture was stirred at 85° C. for 2 h. The reaction was concentrated and purified by flash chromatography on silica gel (EA/PE=0-20%) to give (2R,3R,4S,5S)-4-(benzoyloxy)-2-[(benzoyloxy)methyl]-5-(2-chloro-4-{hexahydro-1H-cyclopenta[c]pyrrol-2-yl}imidazo[1,5-b]pyridazin-7-yl)oxolan-3-yl benzoate (400 mg, 55% yield) as a yellow oil. Mass Spectrum (ESI) m/z=707.7 (M+1).

Step H: To a mixture of (2R,3R,4S,5S)-4-(benzoyloxy)-2-[(benzoyloxy)methyl]-5-(2-chloro-4-{hexahydro-1H-cyclopenta[c]pyrrol-2-yl}imidazo[1,5-b]pyridazin-7-yl)oxolan-3-yl benzoate (400 mg, 0.57 mmol) in MeOH (8 mL) was added K₂CO₃ (160 mg, 1.14 mmol). The mixture was stirred at rt for 3 h. Then the reaction was concentrated and purified by flash chromatography on silica gel (MeOH/DCM=0-10%) to give (2S,3R,4S,5R)-2-(2-chloro-4-{hexahydro-1H-cyclopenta[c]pyrrol-2-yl}imidazo[1,5-b]pyridazin-7-yl)-5-(hydroxymethyl)oxolane-3,4-diol (200 mg, 80% yield) as a yellow solid. Mass Spectrum (ESI) m/z=394.8 (M+1).

Step I: To a solution of (2S,3R,4S,5R)-2-(2-chloro-4-{hexahydro-1H-cyclopenta[c]pyrrol-2-yl}imidazo[1,5-b]pyridazin-7-yl)-5-(hydroxymethyl)oxolane-3,4-diol (200 mg, 0.51 mmol) in acetone (5 mL) was added 2,2-dimethoxypropane (265 mg, 2.55 mmol) and p-toluenesulfonic acid (109 mg, 0.64 mmol). The reaction mixture was stirred for 16 h at rt. After quenching with sat. aq. NaHCO₃ solution, it was extracted with EA, dried over anhydrous Na₂SO₄, filtered, and the filtrate was evaporated in vacuo and purified by silica column chromatography (PE/EA=1:1) to give [(3aR,4R,6S,6aS)-6-(2-chloro-4-{hexahydro-1H-cyclopenta[c]pyrrol-2-yl}imidazo[1,5-b]pyridazin-7-yl)-2,2-dimethyl-tetrahydrofuro[3,4-d][1,3]dioxol-4-yl]methanol (110 mg, 46% yield) as a yellow solid. Mass Spectrum (ESI) m/z=434.8 (M+1).

Step J: To a mixture of [(3aR,4R,6S,6aS)-6-(2-chloro-4-{hexahydro-1H-cyclopenta[c]pyrrol-2-yl}imidazo[1,5-b]pyridazin-7-yl)-2,2-dimethyl-tetrahydrofuro[3,4-d][1,3]dioxol-4-yl]methanol (100 mg, 0.23 mmol) and di-tert-butyl {[(tert-butoxy) (diisopropylamino) phosphanyl]methyl} phosphonate (190 mg, 0.46 mmol) in acetonitrile (2 mL) was added 1H-imidazole-4,5-dicarbonitrile (55 mg, 0.46 mmol) carefully. The reaction was stirred for 12 h at 20° C. Then tert-butyl hydroperoxide (295 mg, 2.3 mmol) was added into the mixture. The reaction was stirred for 2 h at 20° C. Then the reaction was diluted with EA (20 mL). The organic layer was washed with sat. aq. Na₂C₂O₃ solution and brine, dried over Na₂SO₄, filtered and the filtrate was concentrated and purified by flash chromatography on silica gel (DCM/MeOH=10:1) to give di-tert-butyl [({[(3aR,4R,6S,6aS)-6-(2-chloro-4-{hexahydro-1H-cyclopenta[c]pyrrol-2-yl}imidazo[1,5-b]pyridazin-7-yl)-2,2-dimethyl-tetrahydrofuro[3,4-d][1,3]dioxol-4-yl]methoxy}(tert-butoxy)phosphoryl)methyl]phosphonate (100 mg, 60% yield). Mass Spectrum (ESI) m/z=760.6 (M+1).

Step K: To a mixture of di-tert-butyl [({[(3aR,4R,6S,6aS)-6-(2-chloro-4-{hexahydro-1H-cyclopenta[c]pyrrol-2-yl}imidazo[1,5-b]pyridazin-7-yl)-2,2-dimethyl-tetrahydrofuro[3,4-d][1,3]dioxol-4-yl]methoxy}(tert-butoxy)phosphoryl)methyl]phosphonate (100 mg, 0.13 mmol) and Ethylene glycol (40 mg, 0.65 mmol) in dioxane (1 mL) was added hydrochloric acid (0.42 mL, 1.69 mmol) carefully. The reaction was stirred for 1 h at 20° C. The reaction was concentrated and purified by reverse phase preparative HPLC (Daisogel™—C18, 10 um, 250×50 mm) using a gradient of 0.2% formic acid/ACN from 80:20 to 60:40. Suitable fractions were pooled and lyophilized to give (((((2R,3S,4R,5S)-5-(2-chloro-4-(((R)-2,3-dihydro-1H-inden-1-yl)(methyl)amino)imidazo[2,1-f][1,2,4]triazin-7-yl)-3,4-dihydroxytetrahydrofuran-2-yl)methoxy)(hydroxy)phosphoryl)methyl) phosphonic acid (15 mg, 25% yield) as a white solid. ¹H NMR (400 MHz, D₂O) δ 8.08 (s, 1H), 5.47 (s, 1H), 4.58-4.54 (m, 1H), 4.27-4.23 (m, 3H), 4.05-4.00 (m, 2H), 3.65-3.62 (m, 3H), 3.25-3.23 (m, 1H), 2.81-2.79 (m, 2H), 2.15-2.26 (m, 2H), 1.82-1.80 (m, 2H), 1.72-1.70 (m, 1H), 1.60-1.58 (m, 1H), 1.46-1.42 (m, 2H). Mass Spectrum (ESI) m/z=552.6 (M+1).

### Example S10

### Synthesis of (((((2R,3S,4R,5S)-5-(2-chloro-4-(cyclopentylamino)thieno[3,2-d]pyrimidin-7-yl)-3,4-dihydroxytetrahydrofuran-2-yl)methoxy)(hydroxy)phosphoryl)methyl)phosphonic acid (Compound No. 69)

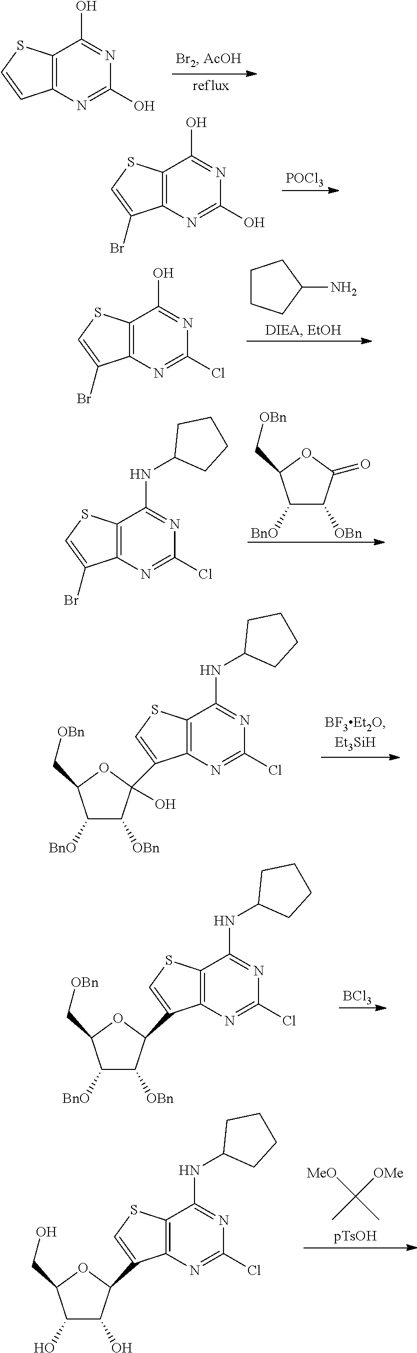

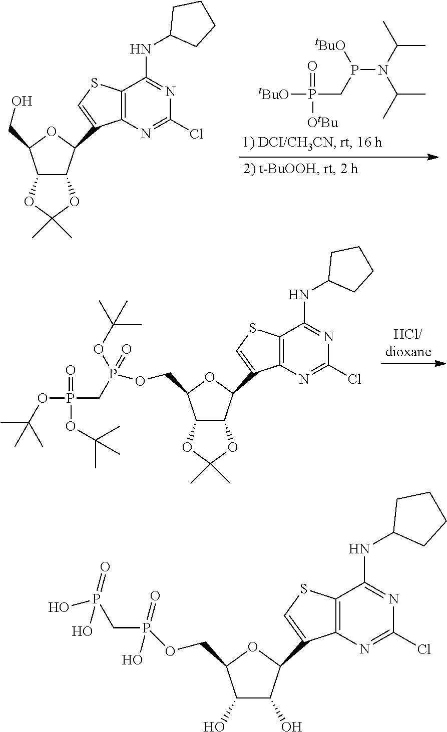 [SMILES: CC1(C)O[C@@H]2[C@H](O1)[C@@H](CO)O[C@H]2c1csc2c(NC3CCCC3)nc(Cl)nc12 ; CC(C)N(C(C)C)P(CP(=O)(OC(C)(C)C)OC(C)(C)C)OC(C)(C)C ; CC(C)(C)OP(=O)(CP(=O)(OC(C)(C)C)OC(C)(C)C)OC[C@H]1O[C@@H](c2csc3c(NC4CCCC4)nc(Cl)nc23)[C@@H]2OC(C)(C)O[C@@H]21 ; O=P(O)(O)CP(=O)(O)OC[C@H]1O[C@@H](c2csc3c(NC4CCCC4)nc(Cl)nc23)[C@H](O)[C@@H]1O]

Step A: To a solution of thieno[3,2-d]pyrimidine-2,4-diol (3 g, 17.8 mmol) in AcOH (30 mL) was added bromine (8.55 g, 53.5 mmol). The reaction was heated to 110° C. and stirred for 48 h. After the reaction was completed, the mixture was poured into ice-water and filtered to give the crude product (4 g, 82% yield) as a white solid. Mass Spectrum (ESI) m/z=247.0 (M+1).

Step B: To a suspension of 7-bromoimidazo[2,1-f][1,2,4]triazine-2,4-diol (4 g, 16.3 mmol) in POCl₃ (20 mL) was added triethylamine hydrochloride (4.5 g, 32.5 mmol) at 0° C. Then the mixture was stirred at 110° C. for 8 h in a sealed tube. The solvent was removed under reduced pressure and the residue was dissolved in DCM (10 mL), poured into ice-water (20 mL). The organic layer was washed with brine, dried, filtered and the filtrate was concentrated and purified by flash chromatography on silica gel to give the product (2.5 g, 55% yield) as a white solid. Mass Spectrum (ESI) m/z=284.8 (M+1).

Step C: To a solution of 7-bromo-2,4-dichloroimidazo[2,1-f][1,2,4]triazine (2 g, 7.1 mmol) in THF (20 mL) was added diethyl(isopropyl)amine (1.8 g, 14.2 mmol) followed by cyclopentanamine (664 mg, 7.81 mmol). The mixture was stirred at rt for 2 h. The reaction was monitored by LCMS, and upon completion, was concentrated and purified by flash chromatography on silica gel to give the product (1.7 g, 71% yield) as a white solid. Mass Spectrum (ESI) m/z=331.9 (M+1).

Step D: To a solution of 7-bromo-2-chloro-N-cyclopentylthieno[3,2-d]pyrimidin-4-amine (1.2 g, 3.63 mmol) in THF (30 mL) was added n-BuLi (2.4 M, 3.6 mL, 8.6 mmol) at −78° C. After the mixture was stirred at −78° C. for 30 min, a solution of (3R,4R,5R)-3,4-bis(benzyloxy)-5-[(benzyloxy)methyl]oxolan-2-one (1.8 g, 4.36 mmol) in THF (10 mL) was added slowly. Then the mixture was stirred at −78° C. for 2 h. Sat. aq. NH₄Cl solution (50 mL) was added, and the mixture was extracted with EA (50 mL×2). The combined organic layers were washed with brine, dried over MgSO₄, filtered, concentrated and purified by flash chromatography on silica gel (24 g, EA/PE=0-21%) to give (3R,4R,5R)-3,4-bis(benzyloxy)-5-((benzyloxy)methyl)-2-(2-chloro-4-(cyclopentylamino)thieno[3,2-d]pyrimidin-7-yl)tetrahydrofuran-2-ol (1.1 g, 45% yield) as a yellow oil. Mass Spectrum (ESI) m/z=673.6 (M+1).

Step E: To a solution of (3R,4R,5R)-3,4-bis(benzyloxy)-5-[(benzyloxy)methyl]-2-[2-chloro-4-(cyclopentylamino)thieno[3,2-d]pyrimidin-7-yl]oxolan-2-ol (1 g, 1.49 mmol) in DCM (10 mL) was added BF₃.OEt₂ (1.06 g, 7.45 mmol) and triethylsilane (866 mg, 7.45 mmol) at −70° C. The mixture was stirred at rt for 1 h. Sat. aq. NaHCO₃ solution was added, and the mixture was extracted with DCM (20 mL×2). The combined organic layers were washed with brine, dried over MgSO₄, filtered, concentrated and purified by flash chromatography on silica gel (12 g, EA/PE=0-21%) to give 7-((2S,3S,4R,5R)-3,4-bis(benzyloxy)-5-((benzyloxy)methyl)tetrahydrofuran-2-yl)-2-chloro-N-cyclopentylthieno[3,2-d]pyrimidin-4-amine (400 mg, 41% yield) as a colorless oil and 7-((2R,3S,4R,5R)-3,4-bis(benzyloxy)-5-((benzyloxy)methyl)tetrahydrofuran-2-yl)-2-chloro-N-cyclopentylthieno[3,2-d]pyrimidin-4-amine (500 mg, 51% yield) as a colorless oil. Mass Spectrum (ESI) m/z=655.6 (M+1).

Step F: To a solution of 7-[(2S,3S,4R,5R)-3,4-bis(benzyloxy)-5-[(benzyloxy)methyl]oxolan-2-yl]-2-chloro-N-cyclopentylthieno[3,2-d]pyrimidin-4-amine (400 mg, 0.61 mmol) in DCM (1.5 mL) was added BCl₃ (1 M in DCM, 6.1 mL, 6.1 mmol) at −70° C. The mixture was stirred at −70° C. for 1 h. Then the reaction was quenched with methanol/chloroform (1:1, 10 mL). After the reaction mixture had warmed to rt, it was neutralized with NH₃ in methanol (10%, 10 mL) and concentrated to give the crude product, which was purified by column chromatography on silica gel (DCM/MeOH=50:1 to 10:1) to give (2S,3R,4S,5R)-2-(2-chloro-4-(cyclopentylamino)thieno[3,2-d]pyrimidin-7-yl)-5-(hydroxymethyl)tetrahydrofuran-3,4-diol (200 mg, 81% yield) as a yellow solid. Mass Spectrum (ESI) m/z=385.7 (M+1).

Step G: To a solution of (2S,3R,4S,5R)-2-[2-chloro-4-(cyclopentylamino)thieno[3,2-d]pyrimidin-7-yl]-5-(hydroxymethyl)oxolane-3,4-diol (200 mg, 0.52 mmol) in acetone (5 mL) was added 4-methylbenzenesulfonic acid (112 mg, 0.65 mmol) and 2,2-dimethoxypropane (1.25 mL), then the mixture was stirred at rt overnight. The reaction was concentrated and purified by flash chromatography on silica gel (MeOH/DCM=0-10%) to give ((3aR,4R,6S,6aS)-6-(2-chloro-4-(cyclopentylamino)thieno[3,2-d]pyrimidin-7-yl)-2,2-dimethyltetrahydrofuro[3,4-d][1,3]dioxol-4-yl)methanol (200 mg, 86% yield) as a colorless oil. Mass Spectrum (ESI) m/z=425.7 (M+1).

Step H: To a solution of ((3aR,4R,6S,6aS)-6-(2-chloro-4-(cyclopentylamino)thieno[3,2-d]pyrimidin-7-yl)-2,2-dimethyltetrahydrofuro[3,4-d][1,3]dioxol-4-yl)methanol (180 mg, 0.42 mmol) in MeCN (3 mL) was added di-tert-butyl{[(diisopropylamino)(methoxy) phosphanyl] methyl} phosphonate (346 mg, 0.84 mmol) and DCI (99 mg, 0.84 mmol). After the mixture was stirred at rt overnight, t-BuOOH (0.54 g, 4.2 mmol) was added and the mixture was stirred for another 1 h. The reaction was diluted with EA (20 mL) and the organic layers were washed with aqueous Na₂CO₃ (10 mL×4), dried over MgSO₄, filtered, concentrated and purified by flash chromatography on silica gel (MeOH/DCM=0-10%) to give di-tert-butyl ((tert-butoxy(((3aR,4R,6S,6aS)-6-(2-chloro-4-(cyclopentylamino)thieno[3,2-d]pyrimidin-7-yl)-2,2-dimethyltetrahydrofuro[3,4-d][1,3]dioxol-4-yl)methoxy)phosphoryl)methyl)phosphonate (170 mg, 51%) as a colorless oil. Mass Spectrum (ESI) m/z=695.4 (M+1).

Step I: To a solution of [(3aR,4R,6S,6aS)-6-[2-chloro-4-(cyclopentylamino)thieno[3,2-d]pyrimidin-7-yl]-2,2-dimethyl-tetrahydrofuro[3,4-d][1,3]dioxol-4-yl]methyl tert-butyl {[bis(tert-butoxy)phosphoryl]methyl}phosphonate (150 mg, 0.2 mmol) in 1,4-dioxane (5 mL) was added 4N HCl (1 mL) and the mixture was stirred at rt for 2 h. Then the reaction was concentrated and purified by reverse phase preparative HPLC (Daisogel™—C18, 10 um, 250×50 mm, 0.2% FA in H₂O/MeCN from 70% to 50%) to give (((((2R,3S,4R,5S)-5-(2-chloro-4-(cyclopentylamino)thieno[3,2-d]pyrimidin-7-yl)-3,4-dihydroxytetrahydrofuran-2-yl)methoxy)(hydroxy)phosphoryl)methyl)phosphonic acid (25 mg, 22% yield) as a white solid. ¹H NMR (400 MHz, D₂O) δ 8.09 (s, 1H), 5.01-5.08 (m, 1H), 4.38-4.30 (m, 1H), 4.30-4.22 (m, 2H), 4.18-4.15 (m, 1H), 4.13-4.00 (m, 2H), 2.35-2.09 (m, 2H), 2.02-1.90 (m, 2H), 1.75-1.45 (m, 6H). Mass Spectrum (ESI) m/z=543.5 (M+1).

### Example S11

### Synthesis of (((((2R,3S,4R,5S)-5-(6-chloro-8-(cyclopentylamino)imidazo[1,2-b]pyridazin-3-yl)-3,4-dihydroxytetrahydrofuran-2-yl)methoxy) (Compound No. 77)

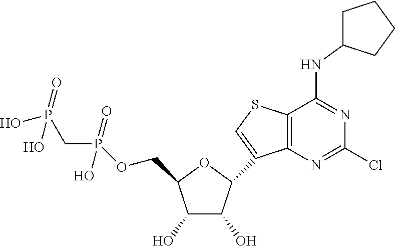 [SMILES: O=P(O)(O)CP(=O)(O)OC[C@H]1O[C@H](c2csc3c(NC4CCCC4)nc(Cl)nc23)[C@H](O)[C@@H]1O]

(((((2R,3S,4R,5S)-5-(6-chloro-8-(cyclopentylamino)imidazo[1,2-b]pyridazin-3-yl)-3,4-dihydroxytetrahydrofuran-2-yl)methoxy) was synthesized by procedures similar to the ones described in Example S10, replacing 7-((2S,3S,4R,5R)-3,4-bis(benzyloxy)-5-((benzyloxy)methyl)tetrahydrofuran-2-yl)-2-chloro-N-cyclopentylthieno[3,2-d]pyrimidin-4-amine in step F with 7-((2R,3S,4R,5R)-3,4-bis(benzyloxy)-5-((benzyloxy)methyl)tetrahydrofuran-2-yl)-2-chloro-N-cyclopentylthieno[3,2-d]pyrimidin-4-amine.

¹H NMR (400 MHz, D₂O) δ 7.23 (s, 1H), 5.07 (d, J=7.0 Hz, 1H), 4.47-4.38 (m, 1H), 4.29-4.24 (m, 1H), 4.24-4.21 (m, 1H), 4.20-4.15 (m, 1H), 4.11-4.00 (m, 2H), 2.24 (t, J=20.1 Hz, 2H), 2.05-1.90 (m, 2H), 1.72-1.47 (m, 6H). Mass Spectrum (ESI) m/z=543.6 (M+1).

### Example S12

### Synthesis of (((((2R,3S,4R)-5-(6-chloro-8-((tetrahydrofuran-3-yl)amino)imidazo[1,2-b]pyridazin-3-yl)-3,4-dihydroxytetrahydrofuran-2-yl)methoxy)(hydroxy)phosphoryl)methyl)phosphonic acid (Compound No. 4)

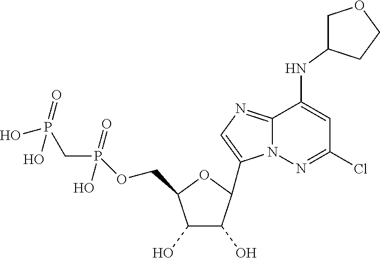 [SMILES: O=P(O)(O)CP(=O)(O)OC[C@H]1OC(c2cnc3c(NC4CCOC4)cc(Cl)nn23)[C@H](O)[C@@H]1O]

The title compound was synthesized by procedures similar to the ones described in Example S2, replacing (S)-1-(2-fluorophenyl)ethan-1-amine in Step A with tetrahydrofuran-3-amine.

¹H NMR (400 MHz, D₂O) δ 8.05 (s, 1H), 6.75 (s, 1H), 5.31-5.26 (m, 1H), 4.53 (t, J=4.9 Hz, 1H), 4.32-4.30 (m, 2H), 4.19-4.17 (m, 1H), 4.05-4.02 (m, 2H), 3.97-3.90 (m, 2H), 3.89-3.84 (m, 2H), 2.36-2.34 (m, 1H), 2.21-2.19 (m, 2H), 2.06-2.00 (m, 1H). Mass Spectrum (ESI) m/z=529.2 (M+1).

### Example S13

### Synthesis of (((((2R,3S,4R)-5-(6-chloro-8-((3,3-difluorocyclopentyl)amino)imidazo[1,2-b]pyridazin-3-yl)-3,4-dihydroxytetrahydrofuran-2-yl)methoxy)(hydroxy)phosphoryl)methyl)phosphonic acid (Compound No. 7)

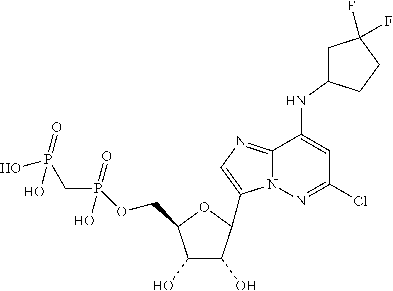 [SMILES: O=P(O)(O)CP(=O)(O)OC[C@H]1OC(c2cnc3c(NC4CCC(F)(F)C4)cc(Cl)nn23)[C@H](O)[C@@H]1O]

The title compound was synthesized by procedures similar to the ones described in Example S2, replacing (S)-1-(2-fluorophenyl)ethan-1-amine in Step A with 3,3-difluorocyclopentan-1-amine.

¹H NMR (400 MHz, D₂O) δ 8.05 (s, 1H), 6.71 (s, 1H), 5.32-5.24 (m, 1H), 4.56-4.49 (m, 1H), 4.36-4.29 (m, 1H), 4.24-4.16 (m, 2H), 4.10-3.98 (m, 2H), 2.71-2.60 (m, 1H), 2.39-2.25 (m, 2H), 2.23-2.09 (m, 4H), 1.93-1.83 (m, 1H). Mass Spectrum (ESI) m/z=562.4 (M+1).

### Example S14

### Synthesis of (((((2R,3S,4R)-5-(8-(benzylamino)-6-chloroimidazo[1,2-b]pyridazin-3-yl)-3,4-dihydroxytetrahydrofuran-2-yl)methoxy)(hydroxy)phosphoryl)methyl)phosphonic acid (Compound No. 16)

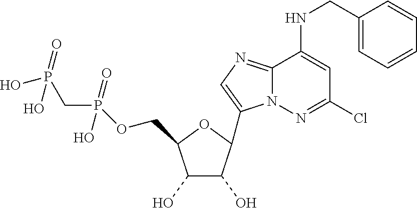 [SMILES: O=P(O)(O)CP(=O)(O)OC[C@H]1OC(c2cnc3c(NCc4ccccc4)cc(Cl)nn23)[C@H](O)[C@@H]1O]

The title compound was synthesized by procedures similar to the ones described in Example S1, replacing cyclopentanamine in Step B with benzylamine.

¹H NMR (400 MHz, D₂O) δ 8.23 (s, 1H), 7.57-7.39 (m, 5H), 6.78 (s, 1H), 5.44 (d, J=5.1 Hz, 1H), 4.78-4.66 (m, 3H), 4.50 (t, J=5.1 Hz, 1H), 4.37-4.30 (m, 1H), 4.25-4.13 (m, 2H), 2.49-2.30 (m, 2H). Mass Spectrum (ESI) m/z=548.5 (M+1).

### Example S15

### Synthesis of (((((2R,3S,4R)-5-(6-chloro-8-((2-chlorobenzyl)amino)imidazo[1,2-b]pyridazin-3-yl)-3,4-dihydroxytetrahydrofuran-2-yl)methoxy)(hydroxy)phosphoryl)methyl)phosphonic acid (Compound No. 17)

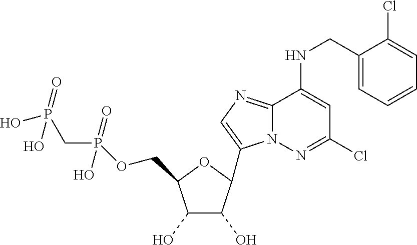 [SMILES: O=P(O)(O)CP(=O)(O)OC[C@H]1OC(c2cnc3c(NCc4ccccc4Cl)cc(Cl)nn23)[C@H](O)[C@@H]1O]

The title compound was synthesized by procedures similar to the ones described in Example S1, replacing cyclopentanamine in Step B with 2-chlorobenzylamine.

¹H NMR (400 MHz, D₂O) δ 8.04 (s, 1H), 7.41 (d, J=7.9 Hz, 1H), 7.34 (d, J=6.4 Hz, 1H), 7.29-7.16 (m, 2H), 6.62 (s, 1H), 5.28 (d, J=4.9 Hz, 1H), 4.68-4.62 (m, 2H), 4.51 (t, J=4.9 Hz, 1H), 4.31 (t, J=5.1 Hz, 1H), 4.19-4.13 (m, 1H), 4.08-3.96 (m, 2H), 2.18 (t, J=20.3 Hz, 2H). Mass Spectrum (ESI) m/z=582.4 (M+1).

### Example S16

### Synthesis of (((((2R,3S,4R)-5-(6-chloro-8-(((S)-1-(3-fluorophenyl)ethyl)amino)imidazo[1,2-b]pyridazin-3-yl)-3,4-dihydroxytetrahydrofuran-2-yl)methoxy)(hydroxy)phosphoryl)methyl)phosphonic acid (Compound No. 19)

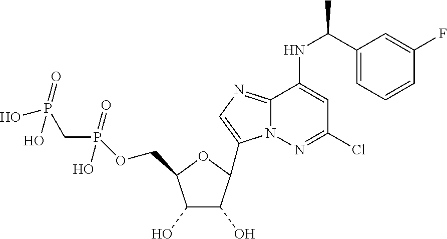 [SMILES: C[C@H](Nc1cc(Cl)nn2c(C3O[C@H](COP(=O)(O)CP(=O)(O)O)[C@@H](O)[C@H]3O)cnc12)c1cccc(F)c1]

The title compound was synthesized by procedures similar to the ones described in Example S1, replacing cyclopentanamine in Step B with (S)-1-(3-fluorophenyl)ethan-1-amine.

¹H NMR (400 MHz, D₂O) δ 8.08 (s, 1H), 7.36-7.28 (m, 1H), 7.21-7.14 (m, 1H), 7.12-7.06 (m, 1H), 7.02-6.94 (m, 1H), 6.35 (s, 1H), 5.28 (d, J=5.1 Hz, 1H), 4.83-4.78 (m, 1H), 4.52 (t, J=5.1 Hz, 1H), 4.32 (t, J=5.1 Hz, 1H), 4.20-4.14 (m, 1H), 4.10-3.96 (m, 2H), 2.25-2.05 (m, 2H), 1.57 (d, J=6.8 Hz, 3H). Mass Spectrum (ESI) m/z=580.5 (M+1).

### Example S17

### Synthesis of (((((2R,3S,4R)-5-(6-chloro-8-(((S)-1-(4-fluorophenyl)ethyl)amino)imidazo[1,2-b]pyridazin-3-yl)-3,4-dihydroxytetrahydrofuran-2-yl)methoxy)(hydroxy)phosphoryl)methyl)phosphonic acid (Compound No. 20)

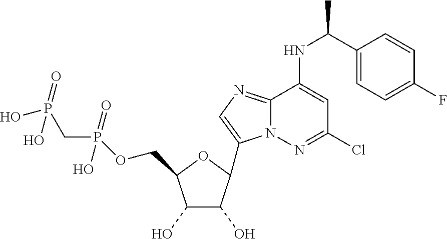 [SMILES: C[C@H](Nc1cc(Cl)nn2c(C3O[C@H](COP(=O)(O)CP(=O)(O)O)[C@@H](O)[C@H]3O)cnc12)c1ccc(F)cc1]

The title compound was synthesized by procedures similar to the ones described in Example S1, replacing cyclopentanamine in Step B with (S)-1-(4-fluorophenyl)ethan-1-amine.

¹H NMR (400 MHz, D₂O) δ 8.07 (s, 1H), 7.40-7.28 (m, 2H), 7.10-6.97 (m, 2H), 6.37 (s, 1H), 5.27 (d, J=5.1 Hz, 1H), 4.82-4.74 (m, 1H), 4.51 (t, J=5.0 Hz, 1H), 4.32 (t, J=5.1 Hz, 1H), 4.21-4.14 (m, 1H), 4.10-3.97 (m, 2H), 2.19 (t, J=20.1 Hz, 2H), 1.55 (d, J=6.7 Hz, 3H). Mass Spectrum (ESI) m/z=580.7 (M+1).

### Example S18

### Synthesis of (((((2R,3S,4R)-5-(6-chloro-8-(((S)-1-phenylethyl)amino)imidazo[1,2-b]pyridazin-3-yl)-3,4-dihydroxytetrahydrofuran-2-yl)methoxy)(hydroxy)phosphoryl)methyl)phosphonic acid (Compound No. 21)

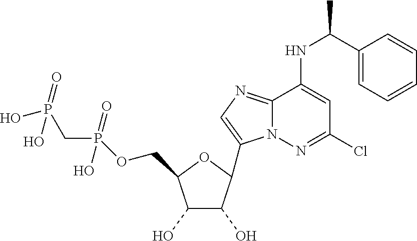 [SMILES: C[C@H](Nc1cc(Cl)nn2c(C3O[C@H](COP(=O)(O)CP(=O)(O)O)[C@@H](O)[C@H]3O)cnc12)c1ccccc1]

The title compound was synthesized by procedures similar to the ones described in Example S1, replacing cyclopentanamine in Step B with (S)-1-phenylethan-1-amine.

¹H NMR (400 MHz, D₂O) δ 8.06 (s, 1H), 7.42-7.29 (m, 4H), 7.29-7.23 (m, 1H), 6.36 (s, 1H), 5.25 (d, J=5.1 Hz, 1H), 4.82-4.75 (m, 1H), 4.50 (t, J=5.0 Hz, 1H), 4.31 (t, J=5.1 Hz, 1H), 4.20-4.14 (m, 1H), 4.11-3.97 (m, 2H), 2.20 (t, J=19.7 Hz, 2H), 1.57 (d, J=6.7 Hz, 3H). Mass Spectrum (ESI) m/z=562.5 (M+1).

### Example S19

### Synthesis of (((((2R,3R,4S)-5-(6-chloro-8-(((S)-1-(2-fluorophenyl)ethyl)amino)imidazo[1,2-b]pyridazin-3-yl)-4-fluoro-3-hydroxytetrahydrofuran-2-yl)methoxy)(hydroxy)phosphoryl)methyl)phosphonic acid (Compound No. 22)

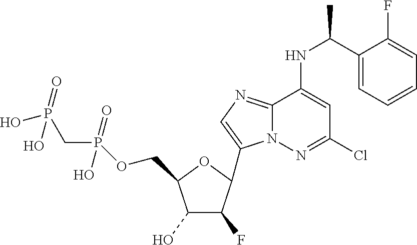 [SMILES: C[C@H](Nc1cc(Cl)nn2c(C3O[C@H](COP(=O)(O)CP(=O)(O)O)[C@@H](O)[C@@H]3F)cnc12)c1ccccc1F]

The title compound was synthesized by procedures similar to the ones described in Example S2, replacing (3R,4R,5R)-3,4-bis(benzyloxy)-5-((benzyloxy)methyl)dihydrofuran-2 (3H)-one in Step B with (3S,4R,5R)-4-(benzyloxy)-5-((benzyloxy)methyl)-3-fluorodihydrofuran-2 (3H)-one.

¹H NMR (400 MHz, D₂O) δ 8 7.96 (s, 1H), 7.37-7.21 (m, 2H), 7.16-7.01 (m, 2H), 6.32 (s, 1H), 5.63-5.53 (m, 1H), 5.35-5.19 (m, 1H), 5.08-4.99 (m, 1H), 4.54-4.45 (m, 1H), 4.22-4.11 (m, 1H), 4.09-3.94 (m, 2H), 2.14 (t, J=20.1 Hz, 2H), 1.60 (d, J=6.3 Hz, 3H). Mass Spectrum (ESI) m/z=582.5 (M+1).

### Example S20

### Synthesis of (((((2R,3S,4R)-5-(6-chloro-8-(pyrrolidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 23)

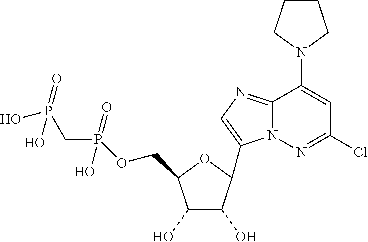 [SMILES: O=P(O)(O)CP(=O)(O)OC[C@H]1OC(c2cnc3c(N4CCCC4)cc(Cl)nn23)[C@H](O)[C@@H]1O]

The title compound was synthesized by procedures similar to the ones described in Example S1, replacing cyclopentanamine in Step B with pyrrolidine.

¹H NMR (400 MHz, D₂O) δ 8.10-7.96 (m, 1H), 6.50 (s, 1H), 5.31-5.22 (m, 1H), 4.54-4.44 (m, 1H), 4.35-4.26 (m, 1H), 4.22-4.13 (m, 1H), 4.13-3.99 (m, 2H), 3.79-3.56 (m, 4H), 2.34-2.12 (m, 2H), 2.06-1.92 (m, 4H). Mass Spectrum (ESI) m/z=512.8 (M+1).

### Example S21

### Synthesis of (((((2R,3R,4S)-5-(6-chloro-8-(pyrrolidin-1-yl)imidazo[1,2-b]pyridazin-3-yl)-4-fluoro-3-hydroxytetrahydrofuran-2-yl)methoxy)(hydroxy)phosphoryl)methyl)phosphonic acid (Compound No. 24)

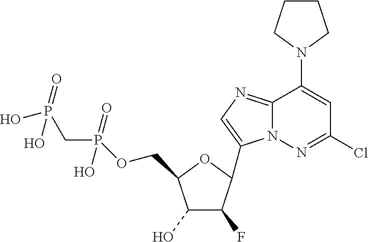 [SMILES: O=P(O)(O)CP(=O)(O)OC[C@H]1OC(c2cnc3c(N4CCCC4)cc(Cl)nn23)[C@@H](F)[C@@H]1O]

The title compound was synthesized by procedures similar to the ones described in Example S1, replacing cyclopentanamine in Step B with pyrrolidine and replacing (3R,4R,5R)-3,4-bis(benzyloxy)-5-((benzyloxy)methyl)dihydrofuran-2 (3H)-one in Step C with (3S,4R,5R)-4-(benzyloxy)-5-((benzyloxy)methyl)-3-fluorodihydrofuran-2 (3H)-one.

¹H NMR (400 MHz, D₂O) δ 8.01 (s, 1H), 6.45 (s, 1H), 5.76-5.66 (m, 1H), 5.50-5.33 (m, 1H), 4.66-4.57 (m, 1H), 4.30-4.24 (m, 1H), 4.16-4.08 (m, 2H), 3.92-3.68 (m, 4H), 2.23 (t, J=20.1 Hz, 2H), 2.14-2.06 (m, 4H). Mass Spectrum (ESI) m/z=512.7 (M−1).

### Example S22

### Synthesis of (((((2R,3R,4S)-5-(6-chloro-8-(3,3-difluoropyrrolidin-1-yl)imidazo[1,2-b]pyridazin-3-yl)-4-fluoro-3-hydroxytetrahydrofuran-2-yl)methoxy)(hydroxy)phosphoryl)methyl)phosphonic acid (Compound No. 28)

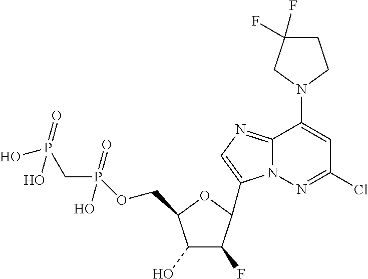 [SMILES: O=P(O)(O)CP(=O)(O)OC[C@H]1OC(c2cnc3c(N4CCC(F)(F)C4)cc(Cl)nn23)[C@@H](F)[C@@H]1O]

The title compound was synthesized by procedures similar to the ones described in Example S1, replacing cyclopentanamine in Step B with 3,3-difluoropyrrolidine and replacing (3R,4R,5R)-3,4-bis(benzyloxy)-5-((benzyloxy)methyl)dihydrofuran-2 (3H)-one in Step C with (3S,4R,5R)-4-(benzyloxy)-5-((benzyloxy)methyl)-3-fluorodihydrofuran-2 (3H)-one.

¹H NMR (400 MHz, D₂O) δ 7 7.86-7.82 (m, 1H), 6.28 (s, 1H), 5.63-5.51 (m, 1H), 5.40-5.21 (m, 1H), 4.56-4.47 (m, 1H), 4.24-4.17 (m, 2H), 4.17-4.14 (m, 1H), 4.08-3.92 (m, 4H), 2.62-2.46 (m, 2H), 2.21 (t, J=20.2 Hz, 2H). Mass Spectrum (ESI) m/z=550.4 (M+1).

### Example S23

### Synthesis of (((((2R,3S,4R)-5-(8-(3-azabicyclo[3.1.0]hexan-3-yl)-6-chloroimidazo[1,2-b]pyridazin-3-yl)-3,4-dihydroxytetrahydrofuran-2-yl)methoxy)(hydroxy)phosphoryl)methyl)phosphonic acid (Compound No. 29)

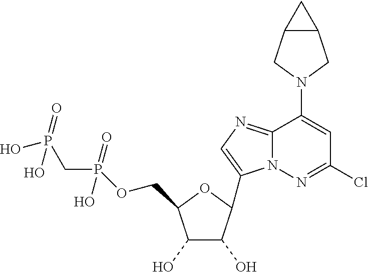 [SMILES: O=P(O)(O)CP(=O)(O)OC[C@H]1OC(c2cnc3c(N4CC5CC5C4)cc(Cl)nn23)[C@H](O)[C@@H]1O]

The title compound was synthesized by procedures similar to the ones described in Example S1, replacing cyclopentanamine in Step B with 3-azabicyclo[3.1.0]hexane.

¹H NMR (400 MHz, D₂O) δ 8.03 (s, 1H), 6.48 (s, 1H), 5.30-5.24 (m, 1H), 4.53-4.47 (m, 1H), 4.33-4.27 (m, 1H), 4.20-4.14 (m, 1H), 4.13-4.07 (m, 1H), 4.06-3.98 (m, 1H), 3.91-3.76 (m, 4H), 2.33-2.17 (m, 2H), 1.83-1.77 (m, 2H), 0.86-0.80 (m, 1H), 0.18-0.12 (m, 1H). Mass Spectrum (ESI) m/z=524.8 (M+1).

### Example S24

### Synthesis of (((((2R,3R,4S)-5-(6-chloro-8-(hexahydrocyclopenta[c]pyrrol-2 (1H)-yl)imidazo[1,2-b]pyridazin-3-yl)-4-fluoro-3-hydroxytetrahydrofuran-2-yl)methoxy)(hydroxy)phosphoryl)methyl)phosphonic acid (Compound No. 31)

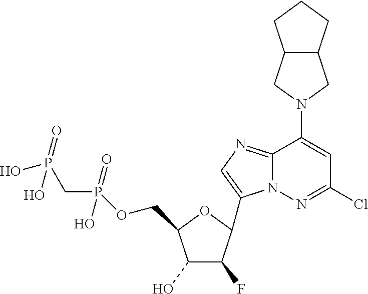 [SMILES: O=P(O)(O)CP(=O)(O)OC[C@H]1OC(c2cnc3c(N4CC5CCCC5C4)cc(Cl)nn23)[C@@H](F)[C@@H]1O]

The title compound was synthesized by procedures similar to the ones described in Example S1, replacing cyclopentanamine in Step B with octahydrocyclopenta[c]pyrrole and replacing (3R,4R,5R)-3,4-bis(benzyloxy)-5-((benzyloxy)methyl)dihydrofuran-2 (3H)-one in Step C with (3S,4R,5R)-4-(benzyloxy)-5-((benzyloxy)methyl)-3-fluorodihydrofuran-2 (3H)-one.

¹H NMR (400 MHz, D₂O) δ 7.95 (s, 1H), 6.37 (s, 1H), 5.67-5.57 (m, 1H), 5.41-5.24 (m, 1H), 4.56-4.43 (m, 1H), 4.21-4.15 (m, 1H), 4.09-3.99 (m, 2H), 3.95-3.85 (m, 2H), 3.61-3.48 (m, 2H), 2.87-2.77 (m, 2H), 2.15 (t, J=20.1 Hz, 2H), 1.90-1.40 (m, 6H). Mass Spectrum (ESI) m/z=553.1 (M−1).

### Example S25

### Synthesis of (((((2R,3S,4R)-5-(6-chloro-8-(indolin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 32)

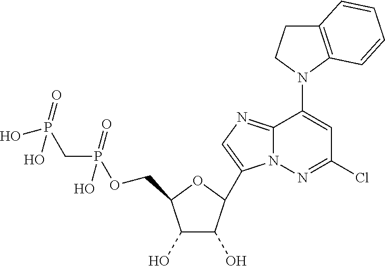 [SMILES: O=P(O)(O)CP(=O)(O)OC[C@H]1OC(c2cnc3c(N4CCc5ccccc54)cc(Cl)nn23)[C@H](O)[C@@H]1O]

The title compound was synthesized by procedures similar to the ones described in Example S1, replacing cyclopentanamine in Step B with indoline.

¹H NMR (400 MHz, D₂O) δ 8.10 (s, 1H), 7.38-7.31 (m, 2H), 7.19-7.14 (m, 1H), 7.11-7.02 (m, 2H), 5.36 (d, J=4.8 Hz, 1H), 4.56 (t, J=4.9 Hz, 1H), 4.36-4.32 (m, 1H), 4.31-4.25 (m, 2H), 4.22-4.18 (m, 1H), 4.14-4.09 (m, 1H), 4.07-4.02 (m, 1H), 3.20 (t, J=7.6 Hz, 2H), 2.25 (t, J=19.6 Hz, 2H). Mass Spectrum (ESI) m/z=560.8 (M+1).

### Example S26

### Synthesis of (((((2R,3S,4R)-5-(6-chloro-8-(piperidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 36)

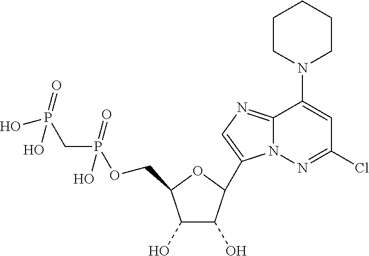 [SMILES: O=P(O)(O)CP(=O)(O)OC[C@H]1OC(c2cnc3c(N4CCCCC4)cc(Cl)nn23)[C@H](O)[C@@H]1O]

The title compound was synthesized by procedures similar to the ones described in Example S1, replacing cyclopentanamine in Step B with piperidine.

¹H NMR (400 MHz, D₂O) δ 8.03 (s, 1H), 6.88 (s, 1H), 5.30-5.28 (m, 1H), 4.51 (t, J=4.9 Hz, 1H), 4.32-4.30 (m, 1H), 4.19-4.17 (m, 1H), 4.05-4.02 (m, 2H), 3.53-3.51 (m, 4H), 2.16-2.14 (m, 2H), 1.64-1.62 (m, 6H). Mass Spectrum (ESI) m/z=527.1 (M+1).

### Example S27

### Synthesis of (((((2R,3S,4R)-5-(6-chloro-8-((S)-2-phenylpiperidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 33)

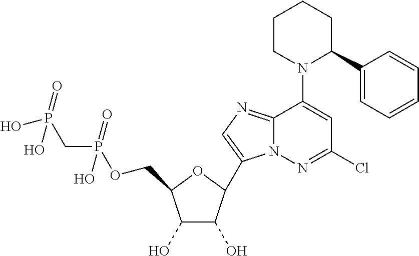 [SMILES: O=P(O)(O)CP(=O)(O)OC[C@H]1OC(c2cnc3c(N4CCCC[C@H]4c4ccccc4)cc(Cl)nn23)[C@H](O)[C@@H]1O]

The title compound was synthesized by procedures similar to the ones described in Example S1, replacing cyclopentanamine in Step B with (S)-2-phenylpiperidine.

¹H NMR (400 MHz, D₂O) δ 7.63 (s, 1H), 7.40-7.34 (m, 2H), 7.28-7.15 (m, 3H), 6.70-6.65 (m, 1H), 6.41 (s, 1H), 5.09 (d, J=6.0 Hz, 1H), 4.86-4.80 (m, 1H), 4.34-4.28 (m, 1H), 4.03-3.80 (m, 4H), 3.24-3.14 (m, 1H), 2.46-2.37 (m, 1H), 2.22 (t, J=20.4 Hz, 2H), 2.06-1.95 (m, 1H), 1.74-1.44 (m, 4H). Mass Spectrum (ESI) m/z=602.6 (M+1).

### Example S28

### Synthesis of (((((2R,3S,4R)-5-(6-chloro-8-(3,4-dihydroisoquinolin-2 (1H)-yl)imidazo[1,2-b]pyridazin-3-yl)-3,4-dihydroxytetrahydrofuran-2-yl)methoxy)(hydroxy)phosphoryl)methyl)phosphonic acid (Compound No. 35)

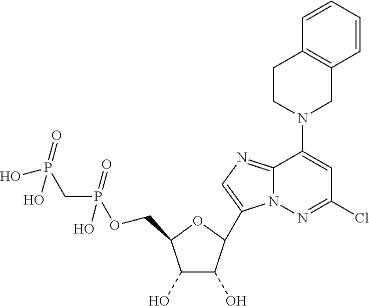 [SMILES: O=P(O)(O)CP(=O)(O)OC[C@H]1OC(c2cnc3c(N4CCc5ccccc5C4)cc(Cl)nn23)[C@H](O)[C@@H]1O]

The title compound was synthesized by procedures similar to the ones described in Example S1, replacing cyclopentanamine in Step B with 1,2,3,4-tetrahydroisoquinoline.

¹H NMR (400 MHz, D₂O) δ 7.71 (s, 1H), 7.24-7.15 (m, 4H), 6.46 (s, 1H), 5.17 (s, 2H), 5.11 (d, J=5.9 Hz, 1H), 4.42-4.35 (m, 2H), 4.34-4.29 (m, 1H), 4.03-3.90 (m, 4H), 3.00-2.95 (m, 2H), 2.21-2.15 (m, 2H). Mass Spectrum (ESI) m/z=574.6 (M+1).

### Example S29

### Synthesis of (((((2R,3S,4R)-5-(6-chloro-8-(4,4-difluoropiperidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 34)

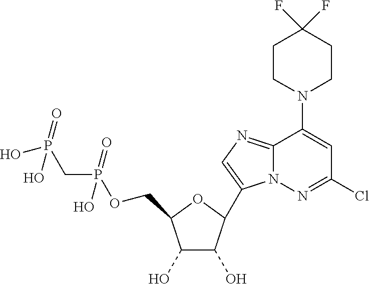 [SMILES: O=P(O)(O)CP(=O)(O)OC[C@H]1OC(c2cnc3c(N4CCC(F)(F)CC4)cc(Cl)nn23)[C@H](O)[C@@H]1O]

The title compound was synthesized by procedures similar to the ones described in Example S1, replacing cyclopentanamine in Step B with 4,4-difluoropiperidine.

¹H NMR (400 MHz, D₂O) δ 7.97 (s, 1H), 6.90 (s, 1H), 5.29-5.26 (m, 1H), 4.50 (t, J=4.7 Hz, 1H), 4.32-4.30 (m, 2H), 4.19-4.17 (m, 1H), 4.05-4.02 (m, 1H), 3.79-3.73 (m, 4H), 2.18-2.16 (m, 6H). Mass Spectrum (ESI) m/z=562.5 (M+1).

### Example S30

### Synthesis of (((((2R,3S,4R)-5-(6-chloro-8-(cyclopentyloxy)imidazo[1,2-b]pyridazin-3-yl)-3,4-dihydroxytetrahydrofuran-2-yl)methoxy)(hydroxy)phosphoryl)methyl)phosphonic acid (Compound No. 3)

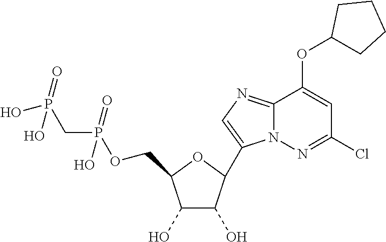 [SMILES: O=P(O)(O)CP(=O)(O)OC[C@H]1OC(c2cnc3c(OC4CCCC4)cc(Cl)nn23)[C@H](O)[C@@H]1O]

The title compound was synthesized by procedures similar to the ones described in Example S1, replacing in Step B the addition of cyclopentanamine in presence of triethylamine in ethanol with cyclopentanol in presence of NaH in THF.

¹H NMR (400 MHz, D₂O) δ 8.20 (s, 1H), 7.24 (s, 1H), 5.35-5.33 (m, 1H), 5.17-5.15 (m, 1H), 4.55-4.53 (m, 1H), 4.34-4.32 (m, 1H), 4.19-4.17 (m, 1H), 4.05-4.04 (m, 2H), 1.92-1.90 (m, 6H), 1.67-1.65 (m, 4H). Mass Spectrum (ESI) m/z=527.8 (M+1).

### Example S31

### Synthesis of (((((2R,3R,4S,5S)-5-(6-chloro-8-(cyclopentylamino)imidazo[1,2-b]pyridazin-3-yl)-4-fluoro-3-hydroxytetrahydrofuran-2-yl)methoxy)(hydroxy)phosphoryl)methyl)phosphonic acid (Compound No. 2)

The title compound was synthesized by procedures similar to the ones described in Example S2, replacing (S)-1-(2-fluorophenyl)ethan-1-amine in Step A with cyclopentanamine.

¹H NMR (400 MHz, D₂O) δ 7.97 (s, 1H), 6.68 (s, 1H), 5.68-5.55 (m, 1H), 5.41-5.22 (m, 1H), 4.55-4.44 (m, 1H), 4.24-4.15 (m, 1H), 4.14-3.94 (m, 3H), 2.48-1.92 (m, 5H), 1.75-1.5 (m, 5H). Mass Spectrum (ESI) m/z=527.1 (M−1).

### Example S32

### Synthesis of (((2-((2R,3S,4R,5S)-5-(6-chloro-8-(cyclopentyl(methyl)amino)imidazo[1,2-b]pyridazin-3-yl)-3,4-dihydroxytetrahydrofuran-2-yl)ethyl)(hydroxy)phosphoryl)methyl)phosphonic acid (Compound No. 10)

The title compound was synthesized by procedures similar to the ones described in Example S2, replacing (S)-1-(2-fluorophenyl)ethan-1-amine in Step A with N-methylcyclopentanamine.

¹H NMR (400 MHz, D₂O) δ 8.03 (s, 1H), 6.72 (s, 1H), 5.29-5.26 (m, 1H), 4.50 (t, J=4.7 Hz, 1H), 4.32-4.30 (m, 2H), 4.19-4.17 (m, 1H), 4.05-4.02 (m, 2H), 3.18 (s, 3H), 2.17-2.15 (m, 2H), 1.97-1.95 (m, 2H), 1.67-1.65 (m, 4H), 1.54-1.52 (m, 2H). Mass Spectrum (ESI) m/z=541.2 (M+1).

### Example S33

### Synthesis of [({[(2R,3S,4R,5S)-5-(8-{bicyclo[2.2.1]heptan-1-ylamino}-6-chloroimidazo[1,2-b]pyridazin-3-yl)-3,4-dihydroxyoxolan-2-yl]methoxy}(hydroxy)phosphoryl)methyl]phosphonic acid (Compound No. 15)

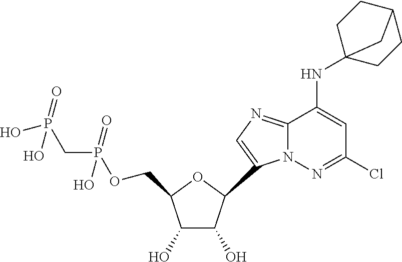 [SMILES: O=P(O)(O)CP(=O)(O)OC[C@H]1O[C@@H](c2cnc3c(NC45CCC(CC4)C5)cc(Cl)nn23)[C@H](O)[C@@H]1O]

The title compound was synthesized by procedures similar to the ones described in Example S2, replacing (S)-1-(2-fluorophenyl)ethan-1-amine in Step A with bicyclo[2.2.1]heptan-1-amine.

¹H NMR (400 MHz, DMSO) δ 7.80-7.75 (m, 2H), 6.39 (s, 1H), 5.08-5.04 (m, 1H), 4.30-4.29 (m, 1H), 4.13-3.96 (m, 4H), 2.19-2.15 (m, 3H), 1.97 (t, J=8.7 Hz, 2H), 1.85-1.70 (m, 6H), 1.45 (t, J=9.2 Hz, 2H). Mass Spectrum (ESI) m/z=553.0 (M+1).

### Example S34

### Synthesis of (((((2R,3S,4R,5S)-5-(6-chloro-8-((S)-2-(2-fluorophenyl)pyrrolidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 26)

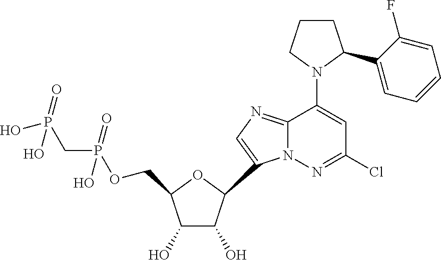 [SMILES: O=P(O)(O)CP(=O)(O)OC[C@H]1O[C@@H](c2cnc3c(N4CCC[C@H]4c4ccccc4F)cc(Cl)nn23)[C@H](O)[C@@H]1O]

The title compound was synthesized by procedures similar to the ones described in Example S2, replacing (S)-1-(2-fluorophenyl)ethan-1-amine in Step A with (S)-2-(2-fluorophenyl)pyrrolidine.

¹H NMR (400 MHz, D₂O) δ 7.96 (s, 1H), 7.28-7.20 (m, 1H), 7.13-7.06 (m, 1H), 7.01-6.98 (m, 2H), 6.07 (s, 1H), 5.35-5.32 (m, 1H), 5.24-5.21 (m, 1H), 4.50-4.48 (m, 1H), 4.31-4.29 (m, 2H), 4.15-4.13 (m, 2H), 3.99-3.97 (m, 2H), 2.42-2.40 (m, 1H), 2.06-2.04 (m, 5H). Mass Spectrum (ESI) m/z=607.2 (M+1).

### Example S35

### Synthesis of (((((2R,3S,4R,5S)-5-(6-chloro-8-(3,3-difluoropyrrolidin-1-yl)imidazo[1,2-b]pyridazin-3-yl)-3,4-dihydroxytetrahydrofuran-2-yl)methoxy)(hydroxy)phosphoryl)methyl)phosphonic acid (Compound No. 27)

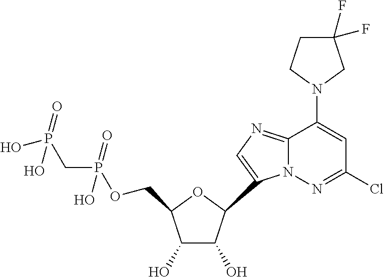 [SMILES: O=P(O)(O)CP(=O)(O)OC[C@H]1O[C@@H](c2cnc3c(N4CCC(F)(F)C4)cc(Cl)nn23)[C@H](O)[C@@H]1O]

The title compound was synthesized by procedures similar to the ones described in Example S2, replacing (S)-1-(2-fluorophenyl)ethan-1-amine in Step A with 3,3-difluoropyrrolidine.

¹H NMR (400 MHz, D₂O) δ 8.04 (s, 1H), 6.55 (s, 1H), 5.29 (d, J=4.7 Hz, 1H), 4.52 (t, J=4.5 Hz, 1H), 4.31 (t, J=4.8 Hz, 1H), 4.22-4.13 (m, 3H), 4.1-4.06 (m, 1H), 4.04-3.97 (m, 3H), 2.65-2.55 (m, 2H), 2.24 (t, J=19.4 Hz, 2H). Mass Spectrum (ESI) m/z=548.5 (M+1).

### Example S36

### Synthesis of (((((2R,3S,4R,5S)-5-(6-cyano-8-(hexahydrocyclopenta[c]pyrrol-2 (1H)-yl)imidazo[1,2-b]pyridazin-3-yl)-3,4-dihydroxytetrahydrofuran-2-yl)methoxy)(hydroxy)phosphoryl)methyl)phosphonic acid (Compound No. 38)

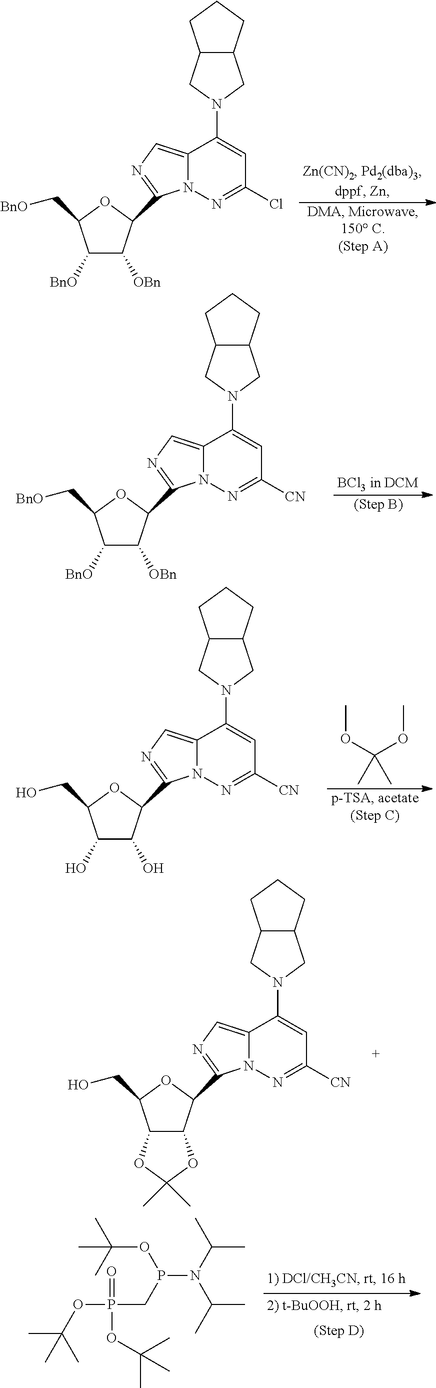

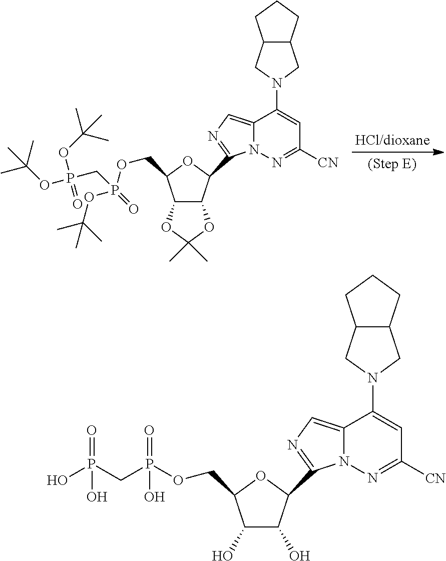 [SMILES: CC(C)(C)OP(=O)(CP(=O)(OC(C)(C)C)OC(C)(C)C)OC[C@H]1O[C@@H](c2ncc3c(N4CC5CCCC5C4)cc(C#N)nn23)[C@@H]2OC(C)(C)O[C@@H]21 ; N#Cc1cc(N2CC3CCCC3C2)c2cnc([C@@H]3O[C@H](COP(=O)(O)CP(=O)(O)O)[C@@H](O)[C@H]3O)n2n1]

Step A: 3-[(2S,3S,4R,5R)-3,4-bis(benzyloxy)-5-[(benzyloxy)methyl]oxolan-2-yl]-6-chloro-8-{hexahydro-1H-cyclopenta[c]pyrrol-2-yl}imidazo[1,2-b]pyridazine was synthesized by procedures similar to the ones described in Example S2, replacing (S)-1-(2-fluorophenyl)ethan-1-amine in Step A with octahydrocyclopenta[c]pyrrole.

Step B: To a 15 mL microwave vial was added 3-[(2S,3S,4R,5R)-3,4-bis(benzyloxy)-5-[(benzyloxy)methyl]oxolan-2-yl]-6-chloro-8-{hexahydro-1H-cyclopenta[c]pyrrol-2-yl}imidazo[1,2-b]pyridazine (500 mg, 0.75 mmol), zinc cyanide (50 mg, 0.45 mmol), Pd₂(dba)₃ (13.8 mg, 0.015 mmol), 1,1′-bis(diphenylphosphino)-ferrocene[dppf] (16.7 mg, 0.03 mmol), zinc dust (0.49 mg, 0.075 mmol) and DMA (5 mL). The reaction vessel was sealed and purged 3 times with nitrogen then heated in a microwave for 20 min at 150° C. After cooling, the suspension was diluted with EA (30 mL) and filtered through a plug of Celite®. To the filtrate was added brine and the layers were separated. The organic layer was washed two additional times with brine, then dried over Na₂SO₄, filtered and the filtrate was concentrated and purified by silica gel column chromotography (PE/EA=3/1) to give 3-[(2S,3S,4R,5R)-3,4-bis(benzyloxy)-5-[(benzyloxy)methyl]oxolan-2-yl]-8-{hexahydro-1H-cyclopenta[c]pyrrol-2-yl}imidazo[1,2-b]pyridazine-6-carbonitrile (390 mg, 78% yield) as a white solid. Mass Spectrum (ESI) m/z=656.4 (M+1).

Step C: To a solution of 3-[(2S,3S,4R,5R)-3,4-bis(benzyloxy)-5-[(benzyloxy)methyl]oxolan-2-yl]-8-{hexahydro-1H-cyclopenta[c]pyrrol-2-yl}imidazo[1,2-b]pyridazine-6-carbonitrile (290 mg, 0.44 mmol) in DCM (10 mL) stirred at −70° C. was added BCl₃ in DCM (4.4 mL of 1M sol., 4.4 mmol) slowly. The reaction mixture was stirred at −70° C. for 1 h. Desired product was found based on LCMS. The reaction was quenched with methanol:chloroform (1:1, 10 mL). After the reaction mixture reached to rt, it was neutralized with NH₃ in methanol (10%, 20 mL) and concentrated to give crude product, which was purified by column chromatography on silica gel (dichloromethane:methanol 50:1 to 5:1) to give 3-((2S,3R,4S,5R)-3,4-dihydroxy-5-(hydroxymethyl)tetrahydrofuran-2-yl)-8-(hexahydrocyclopenta[c]pyrrol-2 (1H)-yl)imidazo[1,2-b]pyridazine-6-carbonitrile (180 mg, 95% yield) as a white solid. Mass Spectrum (ESI) m/z=385.8 (M+1).

Step D: To a solution of 3-[(2S,3R,4S,5R)-3,4-dihydroxy-5-(hydroxymethyl)oxolan-2-yl]-8-{hexahydro-1H-cyclopenta[c]pyrrol-2-yl}imidazo[1,2-b]pyridazine-6-carbonitrile (130 mg, 0.34 mmol) in acetone (2.6 mL) was added 2,2-dimethoxypropane (0.65 mL) and p-toluenesulfonic acid (72.8 mg, 0.42 mmol). The reaction was stirred at rt for 5 h. After quenching with sat. aq. NaHCO₃ solution, the mixture was extracted with EA. The organic layer was dried over anhydrous Na₂SO₄, filtered and the filtrate was concentrated and purified by column chromatography on silica gel (PE:EA=1:3) to give 8-(hexahydrocyclopenta[c]pyrrol-2 (1H)-yl)-3-((3aS,4S,6R,6aR)-6-(hydroxymethyl)-2,2-dimethyltetrahydrofuro[3,4-d][1,3]dioxol-4-yl)imidazo[1,2-b]pyridazine-6-carbonitrile (100 mg, 68% yield) as a white solid. Mass Spectrum (ESI) m/z=426.3 (M+1).

Step E: To a mixture of 3-[(3aS,4S,6R,6aR)-6-(hydroxymethyl)-2,2-dimethyl-tetrahydrofuro[3,4-d][1,3]dioxol-4-yl]-8-{hexahydro-1H-cyclopenta[c]pyrrol-2-yl}imidazo[1,2-b]pyridazine-6-carbonitrile (80 mg, 0.19 mmol) and di-tert-butyl {[(tert-butoxy)(diisopropylamino)phosphanyl]methyl}phosphonate (164.4 mg, 0.38 mmol) in ACN (1.6 mL) was added 1H-imidazole-4,5-dicarbonitrile (44.4 mg, 0.38 mmol) carefully. The reaction was stirred for 6 h at 20° C. Then the reaction was diluted with EA (20 mL). The organic layer was washed with sat. Na₂CO₃ solution and brine. The organic layer was dried over Na₂SO₄, filtered and the filtrate was concentrated. The residue was dissolved in ACN (3 mL), then tert-butyl hydroperoxide (360 mg, 3.8 mmol) was added carefully. The reaction was stirred for 1 h at 20° C. Then the reaction was diluted with EA (25 mL). The organic layer was washed with sat. Na₂CO₃ solution and brine, concentrated and purified by column chromotography obn silica gel (DCM/MeOH=10:1) to give di-tert-butyl ((tert-butoxy(((3aR,4R,6S,6aS)-6-(6-cyano-8-(hexahydrocyclopenta[c]pyrrol-2 (1H)-yl)imidazo[1,2-b]pyridazin-3-yl)-2,2-dimethyltetrahydrofuro[3,4-d][1,3]dioxol-4-yl)methoxy)phosphoryl)methyl)phosphonate (120 mg, 82.3% yield) as a white solid. Mass Spectrum (ESI) m/z=583.6 (M−168+1).

Step F: To a mixture of di-tert-butyl [({[(3aR,4R,6S,6aS)-6-(6-cyano-8-{hexahydro-1H-cyclopenta[c]pyrrol-2-yl}imidazo[1,2-b]pyridazin-3-yl)-2,2-dimethyl-tetrahydrofuro[3,4-d][1,3]dioxol-4-yl]methoxy}(tert-butoxy)phosphoryl)methyl]phosphonate (100 mg, 0.13 mmol) and ethylene glycol (41.5 mg, 0.65 mmol) in dioxane (2.0 mL) was added a solution of HCl in dioxane (0.9 mL, 0.91 mmol) carefully. The reaction was stirred for 1 h at 25° C. Then the reaction was concentrated and purified by prep-HPLC (Daisogel—C18 250×50 mm, 10 um column) using a gradient of 0.2% FA/ACN from 80:20 to 50:50. Suitable fractions were pooled and lyophilized to give (((((2R,3S,4R,5S)-5-(6-cyano-8-(hexahydrocyclopenta[c]pyrrol-2 (1H)-yl)imidazo[1,2-b]pyridazin-3-yl)-3,4-dihydroxytetrahydrofuran-2-yl)methoxy)(hydroxy) phosphoryl)methyl) phosphonic acid (6 mg, 8.1% yield) as a white solid.

¹H NMR (400 MHz, D₂O) δ 7.68 (s, 1H), 6.24 (s, 1H), 5.20-5.19 (m, 1H), 4.55 (t, J=5.9 Hz, 1H), 4.30-4.29 (m, 1H), 4.15-4.13 (m, 1H), 3.94-3.92 (m, 4H), 3.62-3.28 (m, 2H), 2.74-2.72 (m, 2H), 2.01 (t, J=19.9 Hz, 2H), 1.82-1.75 (m, 2H), 1.66-1.65 (m, 1H), 1.55-1.53 (m, 1H), 1.44-1.42 (m, 2H). Spectrum (ESI) m/z=544.2 (M+1).

### Example S37

### Synthesis of (((((2R,3S,4R,5S)-5-(2-chloro-4-(((S)-1-(2-fluorophenyl)ethyl)amino)imidazo[1,5-b]pyridazin-7-yl)-3,4-dihydroxytetrahydrofuran-2-yl)methoxy)(hydroxy)phosphoryl)methyl)phosphonic acid (Compound No. 54)

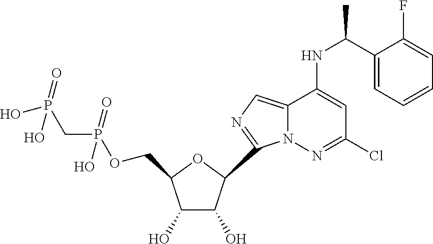 [SMILES: C[C@H](Nc1cc(Cl)nn2c([C@@H]3O[C@H](COP(=O)(O)CP(=O)(O)O)[C@@H](O)[C@H]3O)ncc12)c1ccccc1F]

The title compound was synthesized by procedures similar to the ones described in Example S9, replacing octahydrocyclopenta[c]pyrrole in Step A with (S)-1-(2-fluorophenyl)ethan-1-amine.

¹H NMR (400 MHz, D₂O) δ 8.04 (s, 1H), 7.35-7.20 (m, 2H), 7.12-6.97 (m, 2H), 5.84-5.74 (m, 1H), 5.53-5.47 (m, 1H), 5.08-4.96 (m, 1H), 4.6-4.47 (m, 1H), 4.36-4.15 (m, 3H), 4.07-3.94 (m, 1H), 2.30-1.95 (m, 2H), 1.55 (d, J=6.7 Hz, 3H). Mass Spectrum (ESI) m/z=578.7 (M−1).

### Example S38

### Synthesis of [({[(2R,3S,4R,5S)-5-[2-chloro-4-(pyrrolidin-1-yl)imidazo[1,5-b]pyridazin-7-yl]-3,4-dihydroxyoxolan-2-yl]methoxy}(hydroxy)phosphoryl)methyl]phosphonic acid (Compound No. 59)

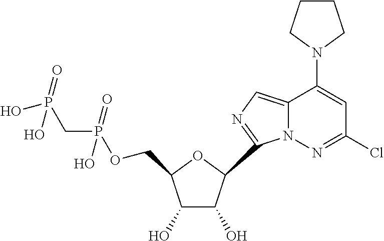 [SMILES: O=P(O)(O)CP(=O)(O)OC[C@H]1O[C@@H](c2ncc3c(N4CCCC4)cc(Cl)nn23)[C@H](O)[C@@H]1O]

The title compound was synthesized by procedures similar to the ones described in Example S9, replacing octahydrocyclopenta[c]pyrrole in Step A with pyrrolidine.

¹H NMR (400 MHz, DMSO) δ 7.90 (s, 1H), 5.69 (s, 1H), 5.27-5.25 (m, 1H), 4.48-4.44 (m, 1H), 4.12-4.10 (m, 2H), 3.98-3.90 (m, 4H), 3.44-3.40 (m, 2H), 2.21-2.18 (m, 2H), 2.01-1.95 (m, 4H). Mass Spectrum (ESI) m/z=512.9 (M+1).

### Example S39

### Synthesis of (((((2R,3S,4R,5S)-5-(4-(3-azabicyclo[3.1.0]hexan-3-yl)-2-chloroimidazo[1,5-b]pyridazin-7-yl)-3,4-dihydroxytetrahydrofuran-2-yl)methoxy)(hydroxy)phosphoryl)methyl)phosphonic acid (Compound No. 61)

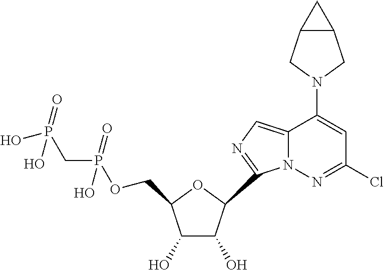 [SMILES: O=P(O)(O)CP(=O)(O)OC[C@H]1O[C@@H](c2ncc3c(N4CC5CC5C4)cc(Cl)nn23)[C@H](O)[C@@H]1O]

The title compound was synthesized by procedures similar to the ones described in Example S9, replacing octahydrocyclopenta[c]pyrrole in Step A with 3-azabicyclo[3.1.0]hexane.

¹H NMR (400 MHz, D₂O) δ 8.07 (s, 1H), 5.81 (s, 1H), 5.48 (d, J=4.9 Hz, 1H), 4.59-4.52 (m, 1H), 4.30-4.20 (m, 3H), 4.05-3.90 (m, 3H), 3.60-3.50 (m, 2H), 2.23-2.09 (m, 2H), 1.83-1.72 (m, 2H), 0.85-0.77 (m, 1H), 0.16-0.09 (m, 1H). Mass Spectrum (ESI) m/z=524.7 [M+H].

### Example S40

### Synthesis of [({[(2R,3S,4R,5S)-5-[2-chloro-4-(2,3-dihydroindol-1-yl)imidazo[1,5-b]pyridazin-7-yl]-3,4-dihydroxyoxolan-2-yl]methoxy}(hydroxy)phosphoryl)methyl]phosphonic acid (Compound No. 64)

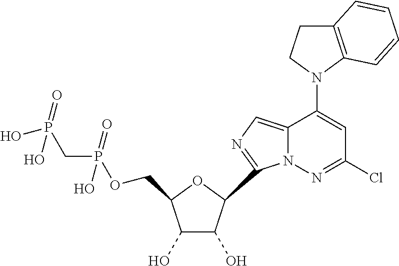 [SMILES: O=P(O)(O)CP(=O)(O)OC[C@H]1O[C@@H](c2ncc3c(N4CCc5ccccc54)cc(Cl)nn23)[C@H](O)[C@@H]1O]

The title compound was synthesized by procedures similar to the ones described in Example S9, replacing octahydrocyclopenta[c]pyrrole in Step A with indoline.

¹H NMR (400 MHz, DMSO) δ 7.79 (s, 1H), 7.44 (d, J=8.1 Hz, 1H), 7.39 (d, J=7.2 Hz, 1H), 7.27-7.20 (m, 1H), 7.07-7.03 (m, 1H), 6.55 (s, 1H), 5.27 (d, J=5.9 Hz, 1H), 4.56-4.53 (m, 1H), 4.49-4.45 (m, 2H), 4.17-4.14 (m, 1H), 4.13-4.08 (m, 1H), 4.02-4.00 (m, 1H), 3.94-3.89 (m, 1H), 3.22-3.20 (m, 2H), 2.18 (t, J=20.4 Hz, 2H). Mass Spectrum (ESI) m/z=560.5 (M+1).

### Example S41

### Synthesis of [({[(2R,3S,4R,5S)-5-(2-chloro-4-{hexahydro-1H-cyclopenta[c]pyrrol-2-yl}thieno[3,2-d]pyrimidin-7-yl)-3,4-dihydroxyoxolan-2-yl]methoxy}(hydroxy)phosphoryl)methyl]phosphonic acid (Compound No. 71)

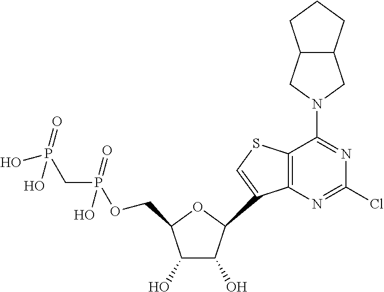 [SMILES: O=P(O)(O)CP(=O)(O)OC[C@H]1O[C@@H](c2csc3c(N4CC5CCCC5C4)nc(Cl)nc23)[C@H](O)[C@@H]1O]

The title compound was synthesized by procedures similar to the ones described in Example S10, replacing cyclopentanamine in Step C with octahydrocyclopenta[c]pyrrole.

¹H NMR (400 MHz, DMSO) δ 7.26 (s, 1H), 5.01-4.96 (m, 1H), 4.06-4.00 (m, 5H), 3.92-3.89 (m, 1H), 3.62-3.56 (m, 2H), 3.16-3.04 (m, 1H), 2.82-2.74 (m, 2H), 2.30-2.13 (m, 2H), 1.86-1.72 (m, 3H), 1.63-1.50 (m, 3H). Mass Spectrum (ESI) m/z=569.5 (M+1).

### Example S42

### Synthesis of [({[(2R,3S,4R,5S)-5-[5-chloro-7-(pyrrolidin-1-yl)thieno[3,2-b]pyridin-3-yl]-3,4-dihydroxyoxolan-2-yl]methoxy}(hydroxy)phosphoryl)methyl]phosphonic acid (Compound No. 74)

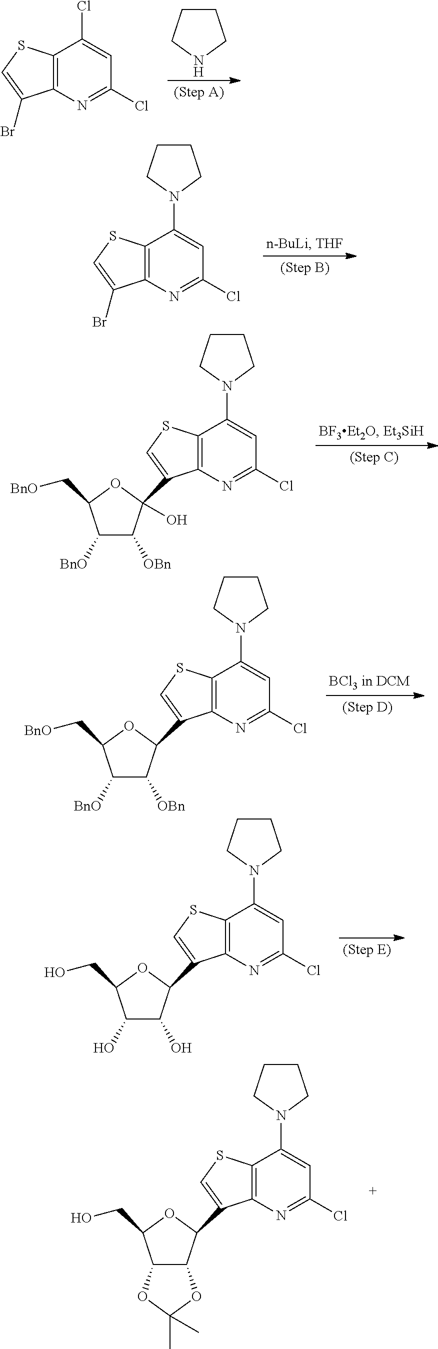

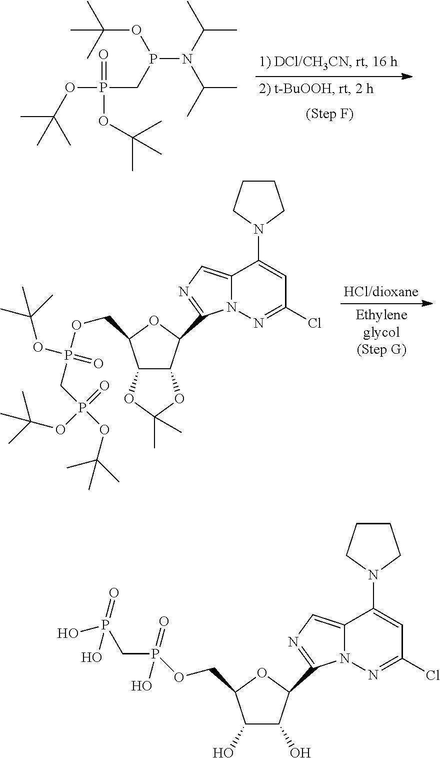 [SMILES: CC(C)N(C(C)C)P(CP(=O)(OC(C)(C)C)OC(C)(C)C)OC(C)(C)C ; CC(C)(C)OP(=O)(CP(=O)(OC(C)(C)C)OC(C)(C)C)OC[C@H]1O[C@@H](c2ncc3c(N4CCCC4)cc(Cl)nn23)[C@@H]2OC(C)(C)O[C@@H]21 ; O=P(O)(O)CP(=O)(O)OC[C@H]1O[C@@H](c2ncc3c(N4CCCC4)cc(Cl)nn23)[C@H](O)[C@@H]1O]

Step A: To a solution of 3-bromo-5,7-dichlorothieno[3,2-b]pyridine (1.2 g, 4.24 mmol) in THF (16 mL) was added N,N-diisopropylethylamine (821 mg, 6.36 mmol) and pyrrolidine (331 mg, 4.66 mmol), then the mixture was stirred at 50° C. overnight. The resulting reaction was concentrated and purified by Flash Chromatography on silica gel (PE/EA=0-20%) to afford 1-{3-bromo-5-chlorothieno[3,2-b]pyridin-7-yl}pyrrolidine (500 mg, 33% yield) as a white solid. Mass Spectrum (ESI) m/z=316.7 (M+1).

Step B: To a solution of 1-{3-bromo-5-chlorothieno[3,2-b]pyridin-7-yl}pyrrolidine (500 mg, 1.57 mmol) in THF (15 mL) was added n-BuLi (0.9 mL, 2.4 mol/L, 2.16 mmol) dropwise at −78° C. under nitrogen. The solution was stirred at this temperature for 0.5 h. Then a solution of (3R,4R,5R)-3,4-bis(benzyloxy)-5-[(benzyloxy)methyl]oxolan-2-one (722 mg, 1.73 mmol) in THF (3 mL) was added. The reaction mixture was stirred at −78° C. for 1 h. Sat. aqueous NH₄Cl solution (10 mL) was added to quench the reaction carefully, and the mixture was extracted with EtOAc. The organic layer was dried over Na₂SO₄, filtered and the filtrate was concentrated. The residue was purified by silica gel column chromatography (hexanes:EtOAc 4:1 to 1:1) to afford (2S,3R,4R,5R)-3,4-bis(benzyloxy)-5-[(benzyloxy)methyl]-2-[5-chloro-7-(pyrrolidin-1-yl)thieno[3,2-b]pyridin-3-yl]oxolan-2-ol (600 mg, 52% yield) as a yellow oil. Mass Spectrum (ESI) m/z=656.9 (M+1).

Step C: To a solution of (2S,3R,4R,5R)-3,4-bis(benzyloxy)-5-[(benzyloxy)methyl]-2-[5-chloro-7-(pyrrolidin-1-yl)thieno[3,2-b]pyridin-3-yl]oxolan-2-ol (700 mg, 1.07 mmol) in DCM (10 mL) under N₂ atmosphere was added BF₃.Et₂O (607 mg, 4.28 mmol) and Et₃SiH (497 mg, 4.28 mmol) at −78° C. successively. The resulting solution was stirred at 25° C. for 2 h. The reaction was quenched with NaHCO₃ and extracted with DCM. The organic layer was concentrated and purified by column chromatography on silica gel (hexanes:EtOAc 5:1 to 1:1) to afford 1-{3-[(2S,3S,4R,5R)-3,4-bis(benzyloxy)-5-[(benzyloxy)methyl]oxolan-2-yl]-5-chlorothieno[3,2-b]pyridin-7-yl}pyrrolidine (650 mg, 85% yield) as a light yellow oil. Mass Spectrum (ESI) m/z=641.1 (M+1).

Step D: To a solution of 1-{3-[(2S,3S,4R,5R)-3,4-bis(benzyloxy)-5-[(benzyloxy)methyl] oxolan-2-yl]-5-chlorothieno[3,2-b]pyridin-7-yl}pyrrolidine (650 mg, 1.01 mmol) in DCM (10 mL) was added BCl₃ in DCM (10 mL, 10 mmol) dropwise at −78° C. under N₂ atmosphere. The mixture was stirred at this temperature for 1 h. The reaction was quenched with methanol:chloroform (1:1, 10 mL). After the reaction mixture reached to rt, it was neutralized with NH₃ in methanol (10%, 20 mL) and concentrated to give crude product, which was purified by column chromatography on silica gel (DCM:methanol=50:1 to 5:1) to give (2S,3R,4S,5R)-2-[5-chloro-7-(pyrrolidin-1-yl)thieno[3,2-b]pyridin-3-yl]-5-(hydroxymethyl)oxolane-3,4-diol (100 mg, 24% yield) as a white solid. Mass Spectrum (ESI) m/z=371.0 (M+1).

Step E: (2S,3R,4S,5R)-2-[5-chloro-7-(pyrrolidin-1-yl)thieno[3,2-b]pyridin-3-yl]-5-(hydroxymethyl)oxolane-3,4-diol (80 mg, 0.215 mmol) was dissolved in acetone (6 mL). 2,2-dimethoxypropane (2 mL) and p-TsOH.H₂O (46 mg, 0.27 mmol) were added. The reaction mixture was stirred at rt overnight, then diluted with EtOAc and carefully quenched with saturated NaHCO₃ (20 mL). The mixture was extracted with EtOAc (3×20 mL). The combined organic layers were dried over MgSO₄, filtered, and the filtrate was concentrated. The residue was purified by column chromatography on silica gel (PE:EA=4:1 to 1:1) to afford [(3aR,4R,6S,6aS)-6-[5-chloro-7-(pyrrolidin-1-yl)thieno[3,2-b]pyridin-3-yl]-2,2-dimethyl-tetrahydrofuro[3,4-d][1,3]dioxol-4-yl]methanol (60 mg, 64% yield) as a light yellow solid. Mass Spectrum (ESI) m/z=411.0 (M+1).

Step F: To a solution of [(3aR,4R,6S,6aS)-6-[5-chloro-7-(pyrrolidin-1-yl)thieno[3,2-b]pyridin-3-yl]-2,2-dimethyl-tetrahydrofuro[3,4-d][1,3]dioxol-4-yl]methanol (78 mg, 0.19 mmol) in MeCN (1.5 mL) was added di-tert-butyl {[(tert-butoxy)(diisopropylamino) phosphanyl]methyl}phosphonate (156 mg, 0.38 mmol) and DCI (45 mg, 0.38 mmol). After the mixture was stirred at rt overnight, t-BuOOH (244 mg, 1.9 mmol) was added and the mixture was stirred for another 1 h. Saturated aqueous Na₂CO₃ solution was added, and the mixture was extracted with DCM. The organic layer was dried over Na₂SO₄, concentrated and purified by column chromatography on silica gel (dichloromethane:methanol=50:1 to 5:1) to give di-tert-butyl [({[(3aR,4R,6S,6aS)-6-[5-chloro-7-(pyrrolidin-1-yl)thieno[3,2-b]pyridin-3-yl]-2,2-dimethyl-tetrahydrofuro[3,4-d][1,3]dioxol-4-yl]methoxy}(tert-butoxy)phosphoryl)methyl] phosphonate (120 mg, 77% yield). Mass Spectrum (ESI) m/z=737.1 (M+1).

Step G: To a mixture of di-tert-butyl [({[(3aR,4R,6S,6aS)-6-[5-chloro-7-(pyrrolidin-1-yl)thieno[3,2-b]pyridin-3-yl]-2,2-dimethyl-tetrahydrofuro[3,4-d][1,3]dioxol-4-yl]methoxy}(tert-butoxy)phosphoryl)methyl]phosphonate (100 mg, 0.14 mmol) in 1,4-dioxane (2 mL) was added a solution of HCl in dioxane (0.8 mL, 4 mmol/L) and ethylene glycol (42 mg, 0.7 mmol) carefully. The reaction was stirred at 25° C. for 2 h. Then the reaction was concentrated and purified by Prep-HPLC (Daisogel—C18 250×50 mm, 10 um column) using a gradient of 0.5% aqueous HCOOH/ACN from 75:25 to 55:45. Suitable fractions were pooled and lyophilized to give the final product [({[(2R,3S,4R,5S)-5-[5-chloro-7-(pyrrolidin-1-yl)thieno[3,2-b]pyridin-3-yl]-3,4-dihydroxyoxolan-2-yl]methoxy}(hydroxy)phosphoryl)methyl]phosphonic acid (9 mg, 10% yield) as a white solid.

¹H NMR (400 MHz, D₂O) δ 8.23 (s, 1H), 6.58 (s, 1H), 5.07 (d, J=5.6 Hz, 1H), 4.35-4.30 (m, 2H), 4.24-4.20 (m, 1H), 4.08-4.00 (m, 2H), 3.81-3.75 (m, 4H), 2.09-2.08 (m, 2H), 2.06-2.00 (m, 4H). Mass Spectrum (ESI) m/z=528.9 (M+1).

### Example S43

### Synthesis of (((((2R,3R,4S,5S)-5-(5-chloro-7-(hexahydrocyclopenta[c]pyrrol-2 (1H)-yl)thieno[3,2-b]pyridin-3-yl)-4-fluoro-3-hydroxytetrahydrofuran-2-yl)methoxy)(hydroxy)phosphoryl)methyl)phosphonic acid (Compound No. 76)

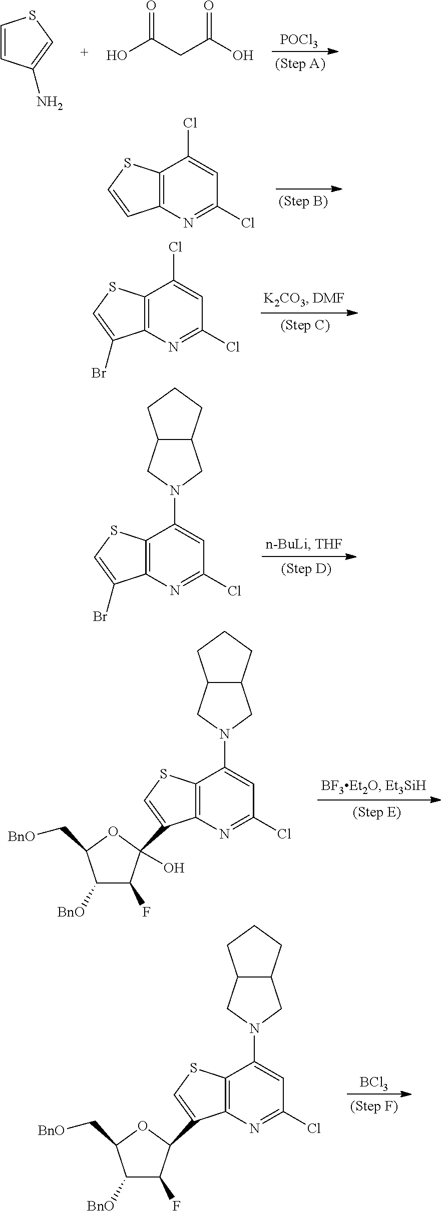

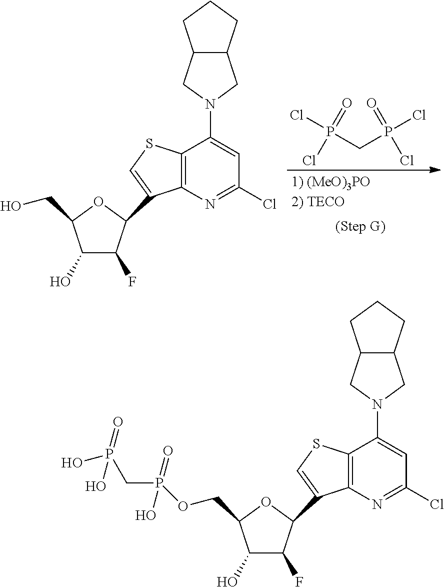 [SMILES: OC[C@H]1O[C@@H](c2csc3c(N4CC5CCCC5C4)cc(Cl)nc23)[C@@H](F)[C@@H]1O ; O=P(Cl)(Cl)CP(=O)(Cl)Cl ; O=P(O)(O)CP(=O)(O)OC[C@H]1O[C@@H](c2csc3c(N4CC5CCCC5C4)cc(Cl)nc23)[C@@H](F)[C@@H]1O]

Step A: A suspension of thiophen-3-amine (3 g, 30.26 mmol) and malonic acid (3.15 g, 30.26 mmol) in POCl₃ (50 mL) was stirred at 100° C. overnight. The reaction was concentrated and quenched with ice water and extracted with DCM. The organic layer was washed with NaHCO₃, concentrated and purified by flash chromatography on silica gel (PE/EA=0-50%) to give 5,7-dichlorothieno[3,2-b]pyridine (1.1 g, 15.7% yield) as a light yellow solid. Mass Spectrum (ESI) m/z=203.8 (M+1).

Step B: To a solution of 5,7-dichlorothieno[3,2-b]pyridine (5.5 g, 26.95 mmol) in AcOH (60 mL) was added Br₂ (2.08 mL, 40.42 mmol). Then the mixture was stirred at 60° C. overnight. The reaction was concentrated, and dissolved with DCM and water, the organic layer was washed with aqueous Na₂S₂O₃ solution, concentrated and purified by Flash chromatography on silica gel (PE/EA=0-20%) to give 3-bromo-5,7-dichlorothieno[3,2-b]pyridine (6 g, 63% yield) as a colourless oil. Mass Spectrum (ESI) m/z=283.8 (M+1).

Step C: To a solution of 3-bromo-5,7-dichlorothieno[3,2-b]pyridine (1.6 g, 5.65 mmol) in DMF (20 mL) was added potassium carbonate (1.56 g, 11.3 mmol) and octahydrocyclopenta[c]pyrrole (691 mg, 6.21 mmol). Then the mixture was stirred at 100° C. for 10 h. The reaction was concentrated and purified by column chromatography (EA/PE=0-50%) to give 3-bromo-5-chloro-7-{hexahydro-1H-cyclopenta[c]pyrrol-2-yl}thieno[3,2-b]pyridine (1 g, 45% yield) as a yellow oil. Mass Spectrum (ESI) m/z=358.9 (M+1).

Step D: To a solution of 3-bromo-5-chloro-7-{hexahydro-1H-cyclopenta[c]pyrrol-2-yl}thieno[3,2-b]pyridine (1 g, 2.8 mmol) in THF (10 mL) was added n-BuLi (2.4 M, 1.4 mL, 3.36 mmol) dropwise, and then a solution of (3S,4R,5R)-4-(benzyloxy)-5-[(benzyloxy)methyl]-3-fluorooxolan-2-one (1.02 g, 3.08 mmol) in THF (4 mL) under nitrogen at −78° C. The reaction mixture was stirred at −78° C. for 1 h. Sat. NH₄Cl solution (20 mL) was added to quench the reaction carefully, then the mixture was extracted with EtOAc. The organic layer was dried over Na₂SO₄, filtered, the filtrate was concentrated and the residue was purified by column chromatography on silica gel (EA/PE=0-50%) to give (2S,3S,4R,5R)-4-(benzyloxy)-5-[(benzyloxy)methyl]-2-(5-chloro-7-{hexahydro-1H-cyclopenta[c]pyrrol-2-yl}thieno[3,2-b]pyridin-3-yl)-3-fluorooxolan-2-ol (800 mg, 42.2% yield) as a yellow oil. Mass Spectrum (ESI) m/z=608.6 (M+1).

Step E: To a solution of (2S,3S,4R,5R)-4-(benzyloxy)-5-[(benzyloxy)methyl]-2-(5-chloro-7-{hexahydro-1H-cyclopenta[c]pyrrol-2-yl}thieno[3,2-b]pyridin-3-yl)-3-fluorooxolan-2-ol (730 mg, 1.2 mmol) in DCM (14 mL) under N₂ atmosphere was added BF₃.Et₂O (1.3 g, 9.6 mmol) and Et₃SiH (1.1 g, 9.6 mol) at −78° C., successively. The resulting solution was stirred at 25° C. for 1 h. The reaction was quenched with NaHCO₃ solution and extracted with DCM. The organic layer was concentrated and purified by column chromatography on silica gel (EA/PE=0-50%) to give 3-[(2S,3R,4R,5R)-4-(benzyloxy)-5-[(benzyloxy)methyl]-3-fluorooxolan-2-yl]-5-chloro-7-{hexahydro-1H-cyclopenta[c]pyrrol-2-yl}thieno[3,2-b]pyridine (350 mg, 44.2% yield) as a yellow oil. Mass Spectrum (ESI) m/z=593.1 (M+1).

Step F: To a solution of 3-[(2S,3R,4R,5R)-4-(benzyloxy)-5-[(benzyloxy)methyl]-3-fluorooxolan-2-yl]-5-chloro-7-{hexahydro-1H-cyclopenta[c]pyrrol-2-yl}thieno[3,2-b]pyridine (300 mg, 0.51 mmol) in DCM (5 mL) was added BCl₃ in DCM (5.1 mL, 5.1 mmol) dropwise at −78° C. under N₂ atmosphere. The mixture was stirred at this temperature for 1 h, then the reaction was quenched with methanol:chloroform (1:1, 20 mL). After the reaction mixture reached to rt, it was neutralized with NH₃ in methanol (10%, 30 mL) and concentrated. The residue was purified by column chromatography on silica gel (DCM/MeOH=0-20%) to give (2R,3R,4S,5S)-5-(5-chloro-7-{hexahydro-1H-cyclopenta[c]pyrrol-2-yl}thieno[3,2-b]pyridin-3-yl)-4-fluoro-2-(hydroxymethyl)oxolan-3-ol (180 mg, 76.9% yield) as a white solid. Mass Spectrum (ESI) m/z=412.8 (M+1).

Step G: To a solution of (2R,3R,4S,5S)-5-(5-chloro-7-{hexahydro-1H-cyclopenta[c]pyrrol-2-yl}thieno[3,2-b]pyridin-3-yl)-4-fluoro-2-(hydroxymethyl)oxolan-3-ol (120 mg, 0.29 mmol) in trimethylphosphate (1.2 mL) at 0° C. was added a cold solution of [(dichlorophosphoryl)methyl]phosphinoyl dichloride (362.2 mg, 1.45 mmol) in trimethylphosphate (0.5 mL) dropwise. Then the reaction solution was stirred at 0° C. for 2 h. TEAC (0.5 M, 2 mL) was added to the reaction carefully and the reaction was stirred at this temperature for 15 mins, then warmed to room temperature and stirring was continued for 1 h. Trimethylphosphate was extracted using MTBE (2 mL×3), and the aqueous layer was basified with ammonium hydroxide to pH=8. The mixture was purified by Prep-HPLC (Daisogel—C18 250×50 mm, 10 um column) using a gradient of 0.02 mol/L TEAC/ACN from 85:15 to 60:40. Suitable fractions were pooled and lyophilized to give the final product (2R,3R,4S,5S)-5-(5-chloro-7-{hexahydro-1H-cyclopenta[c]pyrrol-2-yl}thieno[3,2-b]pyridin-3-yl)-4-fluoro-2-(hydroxymethyl)oxolan-3-ol (10.2 mg, 5.3% yield) as a white solid.

¹H NMR (400 MHz, D₂O) δ 7.27 (s, 1H), 6.46 (s, 1H), 5.61 (d, J=21.8 Hz, 1H), 5.22-5.18 (m, 0.5H), 5.09-5.05 (m, 0.5H), 4.56-4.46 (m, 1H), 4.37-4.29 (m, 1H), 4.08-3.98 (m, 2H), 3.68-3.56 (m, 2H), 2.88-2.75 (m, 2H), 2.23-2.05 (m, 2H), 1.88-1.66 (m, 4H), 1.63-1.40 (m, 4H). Mass Spectrum (ESI) m/z=571.1 (M+1).

### Example S44

### Synthesis of [({[(2R,3R,4S,5S)-5-(2-chloro-4-{hexahydro-1H-cyclopenta[c]pyrrol-2-yl}thieno[3,2-d]pyrimidin-7-yl)-4-fluoro-3-hydroxyoxolan-2-yl]methoxy}(hydroxy)phosphoryl)methyl]phosphonic acid (Compound No. 72)

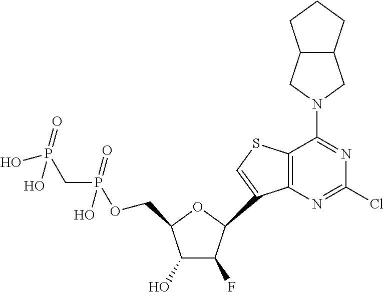 [SMILES: O=P(O)(O)CP(=O)(O)OC[C@H]1O[C@@H](c2csc3c(N4CC5CCCC5C4)nc(Cl)nc23)[C@@H](F)[C@@H]1O]

The title compound was synthesized by procedures similar to the ones described in Example S43, replacing 3-bromo-5-chloro-7-{hexahydro-1H-cyclopenta[c]pyrrol-2-yl}thieno[3,2-b]pyridine in Step D with 7-bromo-2-chloro-4-(hexahydrocyclopenta[c]pyrrol-2 (1H)-yl)thieno[3,2-d]pyrimidine.

¹H NMR (400 MHz, D₂O) δ 7.14 (s, 1H), 5.70-5.52 (m, 1H), 5.26-5.08 (m, 1H), 4.63-4.47 (m, 1H), 4.42-4.25 (m, 1H), 4.22-3.98 (m, 2H), 3.60-3.25 (m, 2H), 2.87-2.58 (m, 2H), 2.30-1.9 (m, 2H), 1.9-1.08 (m, 8H). Mass Spectrum (ESI) m/z=569.6 (M+1).

### BIOLOGICAL EXAMPLES

A variety of assays can be used to evaluate inhibition of compounds for CD73. Compounds of the present disclosure display inhibition of CD73 in the following assays.

### Example B1. CD73 Enzyme Assay

Soluble recombinant CD73 catalyzes the conversion of adenosine monophosphate (AMP) to adenosine and inorganic phosphate. The phosphate detection reagent, PiColorLock™ (Innova Bioscience, Cat #303-0125) is based on the change in absorbance of the dye malachite green in the presence of inorganic phosphate (Pi) and this property can be exploited to measure any enzyme that generates Pi. Recombinant Human 5′-Nucleotidase (CD73) (R&D #5795-EN, CHO derived CD73 (Trp27-Lys547), with a C-terminal 6-His tag) was used in the enzymatic assay. This assay, run in a 384-well plate format (Corning® NBS™ 384 well plates, Cat #3640), is a generic method for measuring inorganic phosphate. The basic assay procedure involves two steps: 1) Enzyme reaction: The CD73 enzyme (R&D #5795-EN) is incubated in the presence or absence of compounds. AMP (sigma, cat #01930) is added to start the kinase reaction. 2) Detection step: Gold mix is added to the assay system, then stabilizers are added. After incubation the absorbance of the solution is read at OD 635 nm. The recorded OD signal is proportional to the enzyme activity.

Briefly, 25 μl human CD73 (0.5 nM final concentration) in the enzymatic buffer solutions (20 mM Tris, 25 mM NaCl, 1 mM MgCl₂, pH 7.5, 0.005% Tween-20) were mixed with various concentrations of the test compound (dissolved in 100% DMSO). These solutions were incubated for 15 min at 25° C., and subsequently 25 μl AMP (30 μM final concentration) was added to start the reaction. The final reaction mixture of enzyme-substrate-compound was incubated for 20 min at 37° C. Meanwhile ‘Gold mix’ was prepared shortly before use by adding 1/100 vol. of accelerator to the PiColorLock™ Gold reagent. 12 μL/well of the ‘Gold mix’ was added to assay plate containing 50 μL enzyme reaction buffer and incubated at 25° C. for 5 min. 5 μL/well stabilizer was added to the assay plate and incubated at 25° C. for 30 min. The absorbance of the well solutions was measured at 635 nm on a Spark 10M instrument (TECAN).

The percent (%) inhibition at each concentration of a compound was calculated relative to the GD value in the Max and Min control wells contained within each assay plate. The Max control wells contained enzyme and substrate as 0% inhibition, and the Min control wells only contained substrate without enzyme as 100% inhibition. The concentrations and percent inhibition values for a test compound are plotted and the concentration of the compound required to achieve 50% inhibition (IC₅₀) was determined with a four-parameter logistic dose response equation. Results for certain compounds are provided in the table 2 below.

TABLE 2

| Example No. | Potency |
|---|---|
| S1 | b |
| S2 | b |
| S3 | b |
| S4 | a |
| S5 | b |
| S5 | b |
| S6 | b |
| S7 | b |
| S8 | b |
| S9 | a |
| S10 | a |
| S11 | c |
| S12 | b |
| S13 | b |
| S14 | b |
| S15 | b |
| S16 | b |
| S17 | a |
| S18 | b |
| S19 | c |
| S20 | b |
| S21 | c |
| S22 | c |
| S23 | a |
| S24 | b |
| S25 | b |
| S26 | b |
| S27 | b |
| S28 | b |
| S29 | b |
| S30 | b |
| S31 | c |
| S32 | b |
| S33 | b |
| S34 | b |
| S35 | b |
| S36 | a |
| S37 | b |
| S38 | b |
| S39 | b |
| S40 | b |
| S41 | c |
| S42 | b |
| S43 | c |
| S44 | c |

“a” means an IC₅₀ of <1 nM;
“b” means an IC₅₀ of 1-9.9 nM;
“c” means an IC₅₀ of 10-99 nM;
and “d” means IC₅₀ of >100 nM

All references throughout, such as publications, patents, patent applications and published patent applications, are incorporated herein by reference in their entireties.

1. A compound of formula (I): 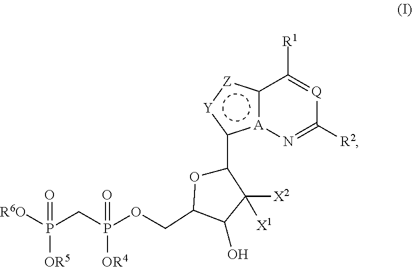
or a stereoisomer, tautomer, prodrug, or a pharmaceutically acceptable salt of any of the foregoing, wherein: 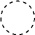
indicates a fully saturated, partially saturated, or aromatic ring;
X¹ and X² are each independently H, —CN, C₁₋₆ alkyl, —OR′, or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;
Q is N or CR³;
Y is CH or N;
Z is CH, O, S, or N, provided that,
when Z is O, S, or N, then Y is CH,
when Z is CH, then Y is N, and
when Z is CH, O, or N, then Q is CR³;
A is C or N;
R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl of R^{1a} and R^{1b} are each independently optionally substituted with R⁷, or
R^{1a} and R^{1b} are taken together with the nitrogen atom to which they attach to form a 3- to 12-membered heterocyclyl optionally substituted with C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, C₆₋₁₄ aryl, halogen, hydroxyl, C₁₋₆ alkoxy, or —CN, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently substituted with C₁₋₆ alkyl, halogen, hydroxyl, C₁₋₆ alkoxy, or —CN;
R² is H, C₁₋₆ alkyl, C₂₋₆ alkenyl, C₂₋₆ alkynyl, halogen, —CN, —NR^{2a}R^{2b}, —OR^{2a}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl of R² are each independently optionally substituted with R⁸, and wherein:
R^{2a} and R^{2b} are each independently H, C₁₋₆ alkyl, C₂₋₆ alkenyl, C₂₋₆ alkynyl, C₃-12 cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R³ is H, C₁₋₆ alkyl, C₁₋₆ haloalkyl, halogen, or —CN;
R⁴, R⁵, and R⁶ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
each R⁷ is independently oxo, C₁₋₆ alkyl, C₂₋₆ alkenyl, C₂₋₆ alkynyl, halogen, —CN, —OR^{7a}, —SR^{7a}, —NR^{7a}R^{7b}, —NO₂, —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein the C₁₋₆ alkyl, C₃₋₆ cycloalkyl, 3- to 12-membered heterocyclyl, 5- to 10-membered heteroaryl, and C₆₋₁₄ aryl of R⁷ are each independently optionally substituted with C₁₋₆ alkyl, C₂₋₆ alkenyl, C₂₋₆ alkynyl, halogen, hydroxyl, C₁₋₆ alkoxy, or —CN, and wherein:
R^{7a} and R^{7b} are each independently H, C₁₋₆ alkyl, C₂₋₆ alkenyl, C₂₋₆ alkynyl, C₃-12 cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
each R⁸ is independently oxo, C₁₋₆ alkyl, C₂₋₆ alkenyl, C₂₋₆ alkynyl, halogen, —CN, —OR^{a}, —SR^{a}, —NR^{8a}R^{8b}, —NO₂, —C═NH(OR^{8a}), —C(O)R^{8a}, —OC(O)R^{8a}, —C(O)OR^{8a}, —C(O)NR^{8a}R^{8b}, —OC(O)NR^{8a}R^{8b}, —NR^{8a}C(O)R^{8b}, —NR^{8a}C(O)OR^{8b}, —S(O)R^{8a}, —S(O)₂R^{8a}, —NR^{8a}S(O)R^{8b}, —C(O)NR^{8a}S(O)R^{8b}, —NR^{8a}S(O)₂R^{8b}, —C(O)NR^{8a}S(O)₂R^{8b}, —S(O)NR^{8a}R^{8b}, —S(O)₂NR^{8a}R^{8b}, —P(O)(OR^{8a}) (OR^{8b}), C₃₋₆ cycloalkyl, 3- to 12-membered heterocyclyl, 5- to 10-membered heteroaryl, or C₆₋₁₄ aryl, wherein:
R^{8a} and R^{8b} are each independently H, C₁₋₆ alkyl, C₂₋₆ alkenyl, C₂₋₆ alkynyl, C₃-12 cycloalkyl, 3- to 12-membered heterocyclyl, 5- to 10-membered heteroaryl, or C₆₋₁₄ aryl, or
R^{8a} and R^{8b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

2. The compound of claim 1, or a stereoisomer, tautomer, prodrug, or a pharmaceutically acceptable salt of any of the foregoing, wherein Z is CH.

3. The compound of claim 1, or a stereoisomer, tautomer, prodrug, or a pharmaceutically acceptable salt of any of the foregoing, wherein Z is 0.

4. The compound of claim 1, or a stereoisomer, tautomer, prodrug, or a pharmaceutically acceptable salt of any of the foregoing, wherein Z is N.

5. The compound of claim 1, or a stereoisomer, tautomer, prodrug, or a pharmaceutically acceptable salt of any of the foregoing, wherein Z is S.

6. The compound of claim 1, or a stereoisomer, tautomer, prodrug, or a pharmaceutically acceptable salt of any of the foregoing, wherein A is N.

7. The compound of claim 1, or a stereoisomer, tautomer, prodrug, or a pharmaceutically acceptable salt of any of the foregoing, wherein A is C.

8. The compound of claim 1, or a stereoisomer, tautomer, prodrug, or a pharmaceutically acceptable salt of any of the foregoing, wherein Q is CR³.

9. The compound of claim 5, or a stereoisomer, tautomer, prodrug, or a pharmaceutically acceptable salt of any of the foregoing, wherein Q is N.

10. The compound of claim 1, or a stereoisomer, tautomer, prodrug, or a pharmaceutically acceptable salt of any of the foregoing, wherein the compound is of formula (II):

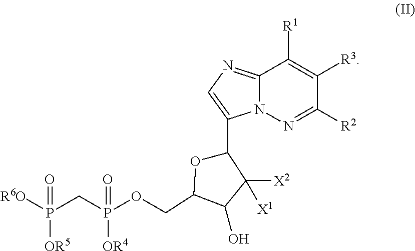

11. The compound of claim 1, or a stereoisomer, tautomer, prodrug, or a pharmaceutically acceptable salt of any of the foregoing, wherein the compound is of formula (III):

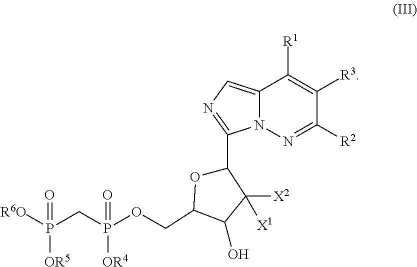

12. The compound of claim 1, or a stereoisomer, tautomer, prodrug, or a pharmaceutically acceptable salt of any of the foregoing, wherein the compound is of formula (IV):

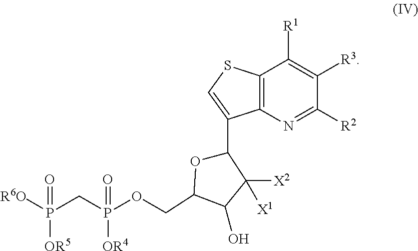

13. The compound of claim 1, or a stereoisomer, tautomer, prodrug, or a pharmaceutically acceptable salt of any of the foregoing, wherein the compound is of formula (V):

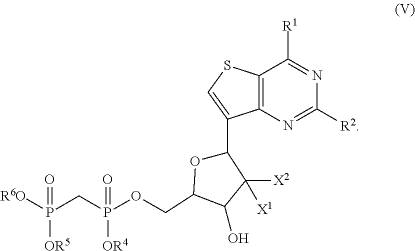

14. The compound of claim 1, or a stereoisomer, tautomer, prodrug, or a pharmaceutically acceptable salt of any of the foregoing, wherein X¹ is H or —OH.

15. The compound of claim 1, or a stereoisomer, tautomer, prodrug, or a pharmaceutically acceptable salt of any of the foregoing, wherein X² is H or halogen.

16. The compound of claim 1, or a stereoisomer, tautomer, prodrug, or a pharmaceutically acceptable salt of any of the foregoing, wherein R¹ is —NR^{1a}R^{1b}.

17. The compound of claim 1, or a stereoisomer, tautomer, prodrug, or a pharmaceutically acceptable salt of any of the foregoing, wherein R¹ is —OR^{1a}.

18. The compound of claim 1, or a stereoisomer, tautomer, prodrug, or a pharmaceutically acceptable salt of any of the foregoing, wherein R^{1a} is C₁₋₆ alkyl, C₃₋₁₂ cycloalkyl, or 3- to 12-membered heterocyclyl, each of which is independently optionally substituted with R⁷.

19. The compound of claim 1, or a stereoisomer, tautomer, prodrug, or a pharmaceutically acceptable salt of any of the foregoing, wherein R^{1a} is 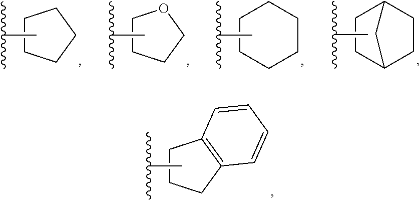
methyl, or ethyl, each of which is optionally substituted with R⁷.

20. The compound of claim 18, or a stereoisomer, tautomer, prodrug, or a pharmaceutically acceptable salt of any of the foregoing, wherein R⁷ is halogen or phenyl optionally substituted with halogen.

21. The compound of claim 1, or a stereoisomer, tautomer, prodrug, or a pharmaceutically acceptable salt of any of the foregoing, wherein R^{1a} is

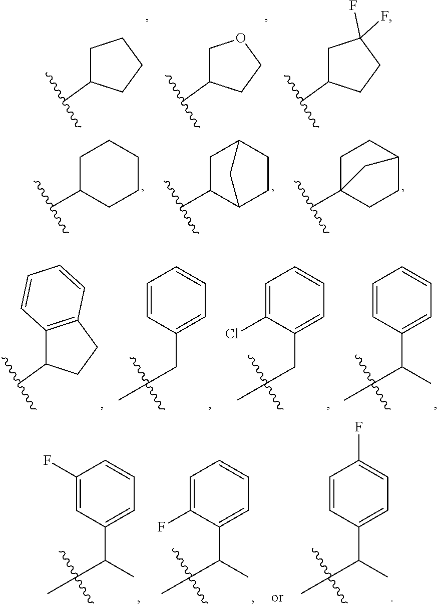

22. The compound of claim 1, or a stereoisomer, tautomer, prodrug, or a pharmaceutically acceptable salt of any of the foregoing, wherein R^{1b} is H or C₁₋₆ alkyl.

23. The compound of claim 1, or a stereoisomer, tautomer, prodrug, or a pharmaceutically acceptable salt of any of the foregoing, wherein R^{1a} and R^{1b} are taken together with the nitrogen atom to which they attach to form a 3- to 12-membered heterocyclyl optionally substituted with C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, C₆₋₁₄ aryl, halogen, hydroxyl, C₁₋₆ alkoxy, or —CN, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently substituted with C₁₋₆ alkyl, halogen, hydroxyl, C₁₋₆ alkoxy, or —CN.

24. The compound of claim 23, or a stereoisomer, tautomer, prodrug, or a pharmaceutically acceptable salt of any of the foregoing, wherein R^{1a} and R^{1b} are taken together with the nitrogen atom to which they attach to form a moiety selected from the group consisting of: 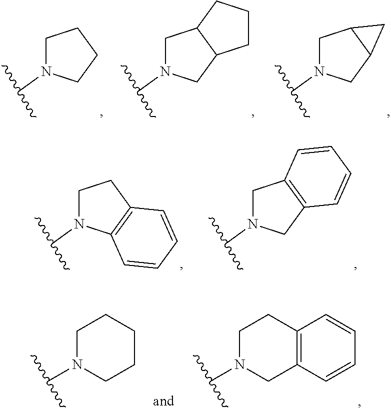
each of which is optionally substituted with halogen or phenyl optionally substituted with halogen.

25. The compound of claim 24, or a stereoisomer, tautomer, prodrug, or a pharmaceutically acceptable salt of any of the foregoing, wherein R^{1a} and R^{1b} are taken together with the nitrogen atom to which they attach to form a moiety selected from the group consisting of:

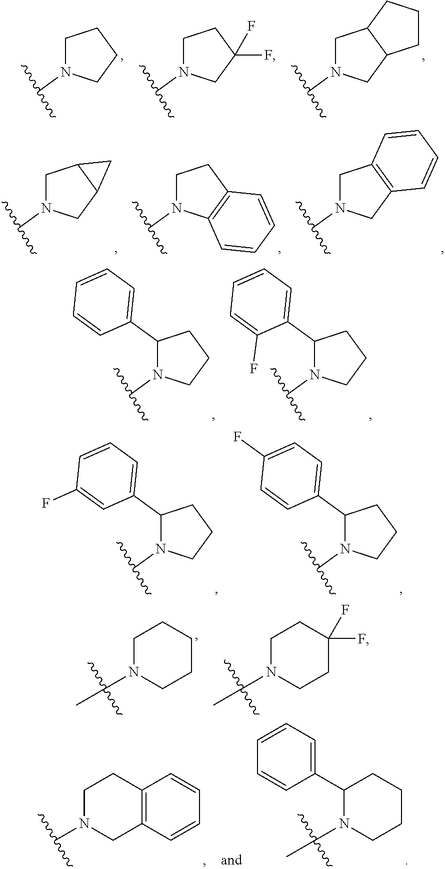

26. The compound of claim 1, or a stereoisomer, tautomer, prodrug, or a pharmaceutically acceptable salt of any of the foregoing, wherein R¹ is selected from the group consisting of

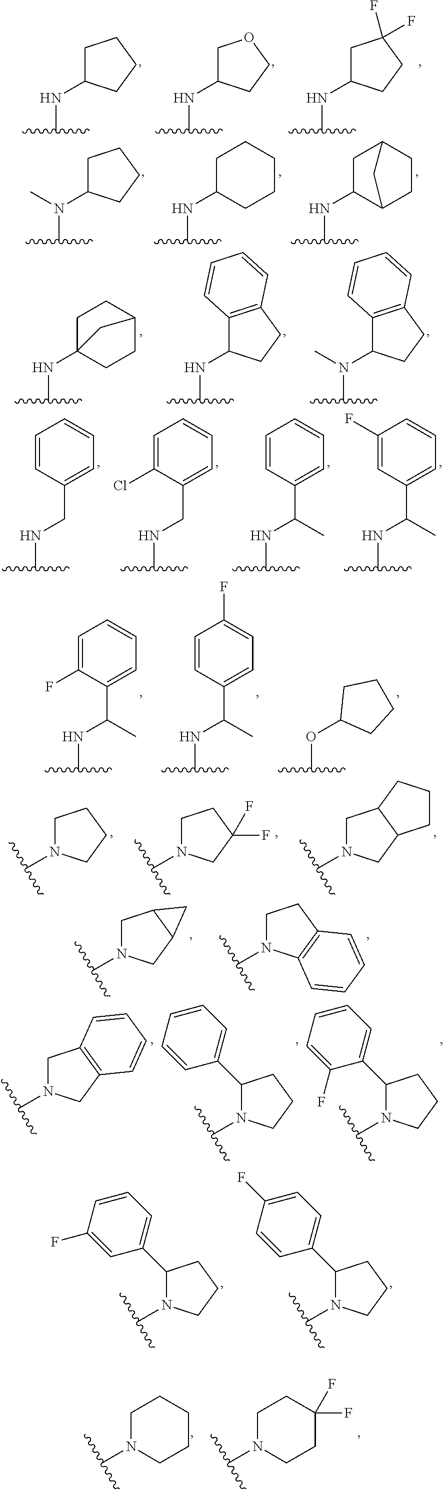

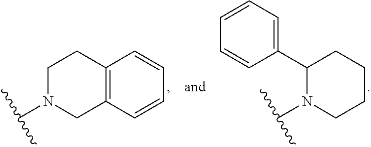 [SMILES: *N1CCc2ccccc2C1 ; *N1CCCCC1c1ccccc1]

27. The compound of claim 1, or a stereoisomer, tautomer, prodrug, or a pharmaceutically acceptable salt of any of the foregoing, wherein R² is H, —CN, or halogen.

28. The compound of claim 1, or a stereoisomer, tautomer, prodrug, or a pharmaceutically acceptable salt of any of the foregoing, wherein R³ is H.

29. The compound of claim 1, or a stereoisomer, tautomer, prodrug, or a pharmaceutically acceptable salt of any of the foregoing, wherein R⁴ is H.

30. The compound of claim 1, or a stereoisomer, tautomer, prodrug, or a pharmaceutically acceptable salt of any of the foregoing, wherein R⁵ is H.

31. The compound of claim 1, or a stereoisomer, tautomer, prodrug, or a pharmaceutically acceptable salt of any of the foregoing, wherein R⁶ is H.

32. A compound selected from the group consisting of 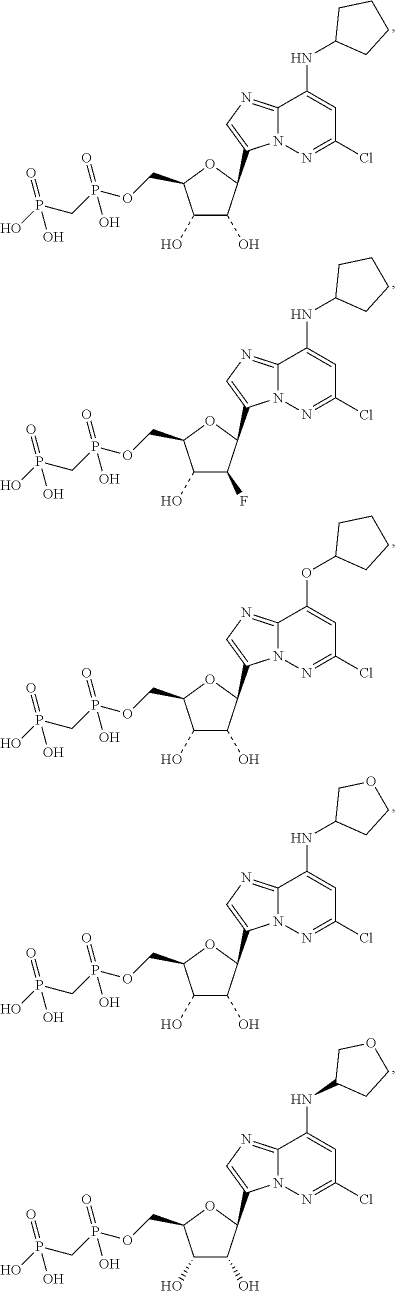 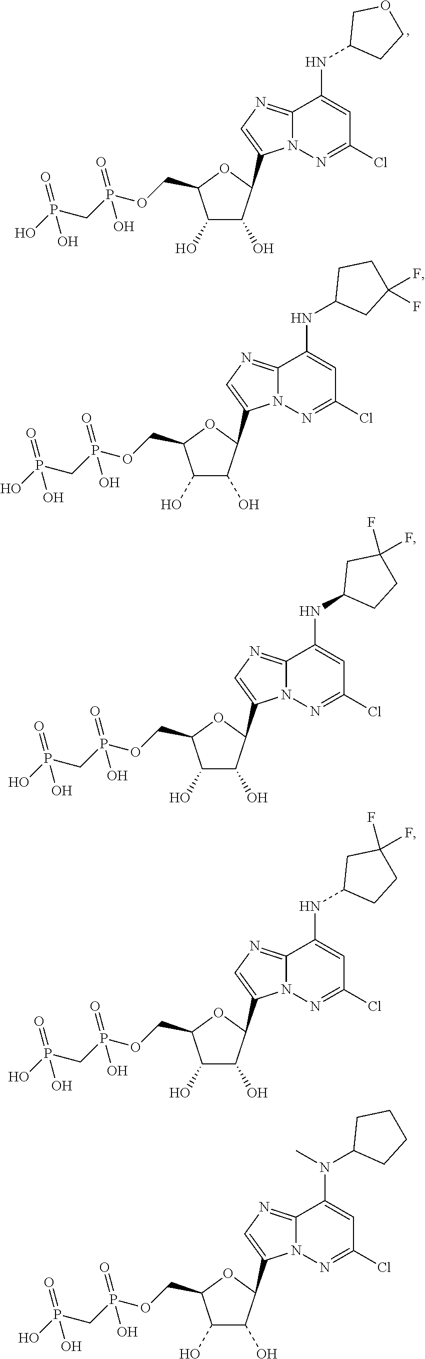 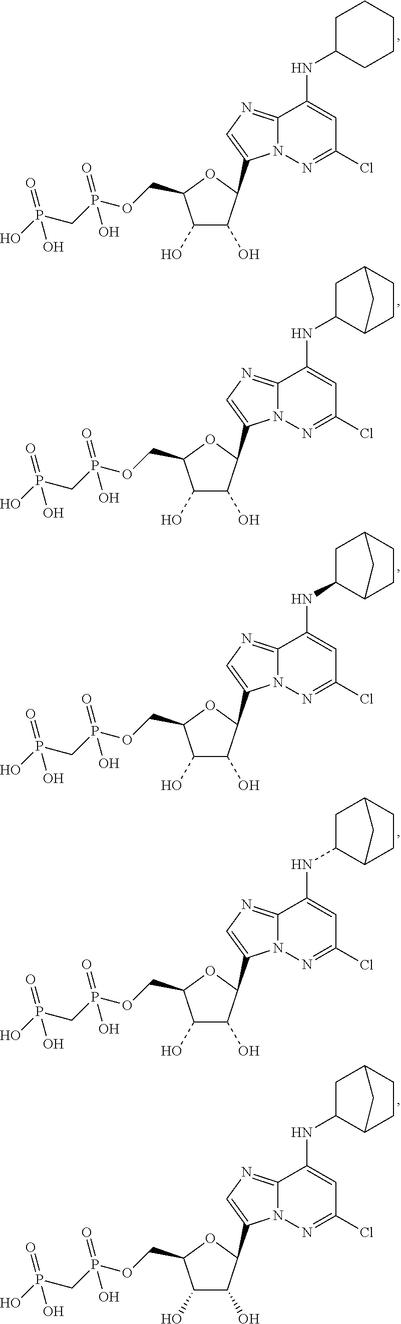 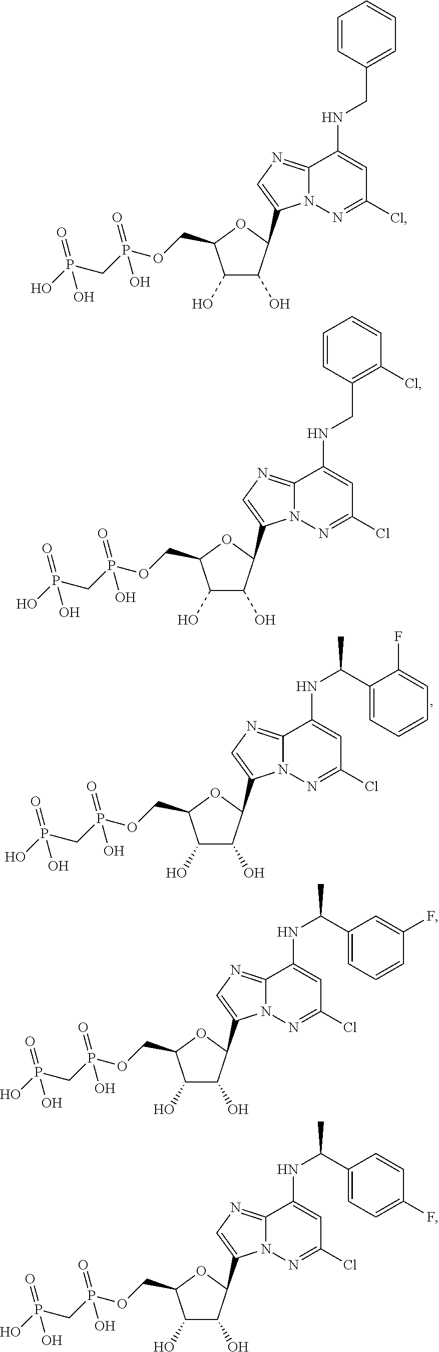 [SMILES: O=P(O)(O)CP(=O)(O)OC[C@H]1O[C@@H](c2cnc3c(NCc4ccccc4)cc(Cl)nn23)[C@H](O)[C@@H]1O ; O=P(O)(O)CP(=O)(O)OC[C@H]1O[C@@H](c2cnc3c(NCc4ccccc4Cl)cc(Cl)nn23)[C@H](O)[C@@H]1O ; C[C@H](Nc1cc(Cl)nn2c([C@@H]3O[C@H](COP(=O)(O)CP(=O)(O)O)[C@@H](O)[C@H]3O)cnc12)c1ccccc1F ; C[C@H](Nc1cc(Cl)nn2c([C@@H]3O[C@H](COP(=O)(O)CP(=O)(O)O)[C@@H](O)[C@H]3O)cnc12)c1cccc(F)c1 ; C[C@H](Nc1cc(Cl)nn2c([C@@H]3O[C@H](COP(=O)(O)CP(=O)(O)O)[C@@H](O)[C@H]3O)cnc12)c1ccc(F)cc1] 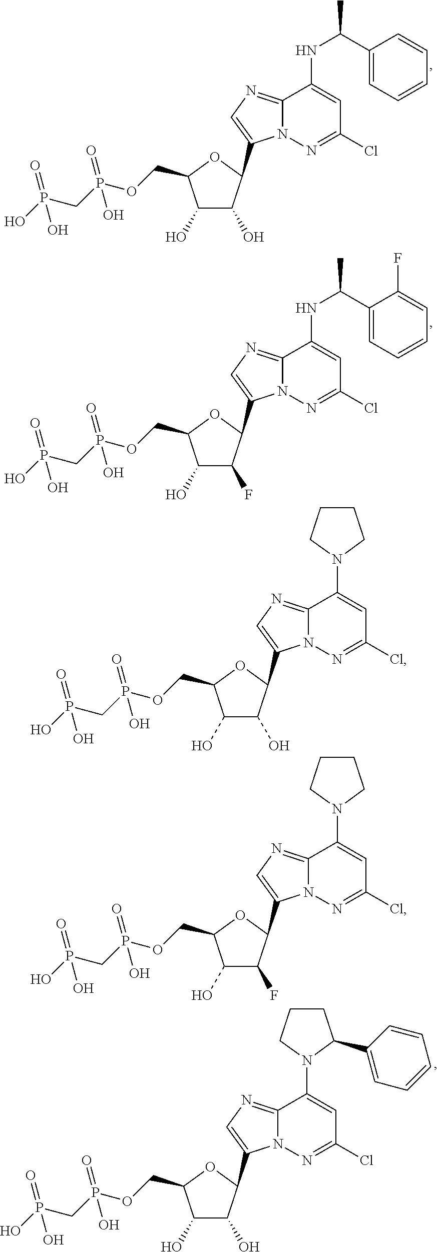 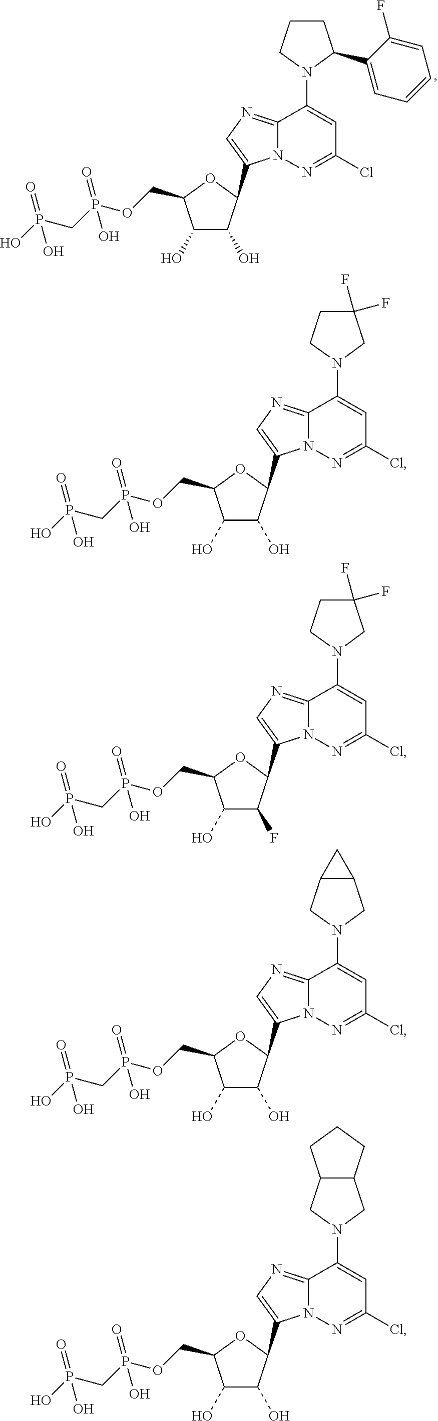 [SMILES: O=P(O)(O)CP(=O)(O)OC[C@H]1O[C@@H](c2cnc3c(N4CCC[C@H]4c4ccccc4F)cc(Cl)nn23)[C@H](O)[C@@H]1O ; O=P(O)(O)CP(=O)(O)OC[C@H]1O[C@@H](c2cnc3c(N4CCC(F)(F)C4)cc(Cl)nn23)[C@H](O)[C@@H]1O ; O=P(O)(O)CP(=O)(O)OC[C@H]1O[C@@H](c2cnc3c(N4CCC(F)(F)C4)cc(Cl)nn23)[C@@H](F)[C@@H]1O ; O=P(O)(O)CP(=O)(O)OC[C@H]1O[C@@H](c2cnc3c(N4CC5CC5C4)cc(Cl)nn23)[C@H](O)[C@@H]1O ; O=P(O)(O)CP(=O)(O)OC[C@H]1O[C@@H](c2cnc3c(N4CC5CCCC5C4)cc(Cl)nn23)[C@H](O)[C@@H]1O] 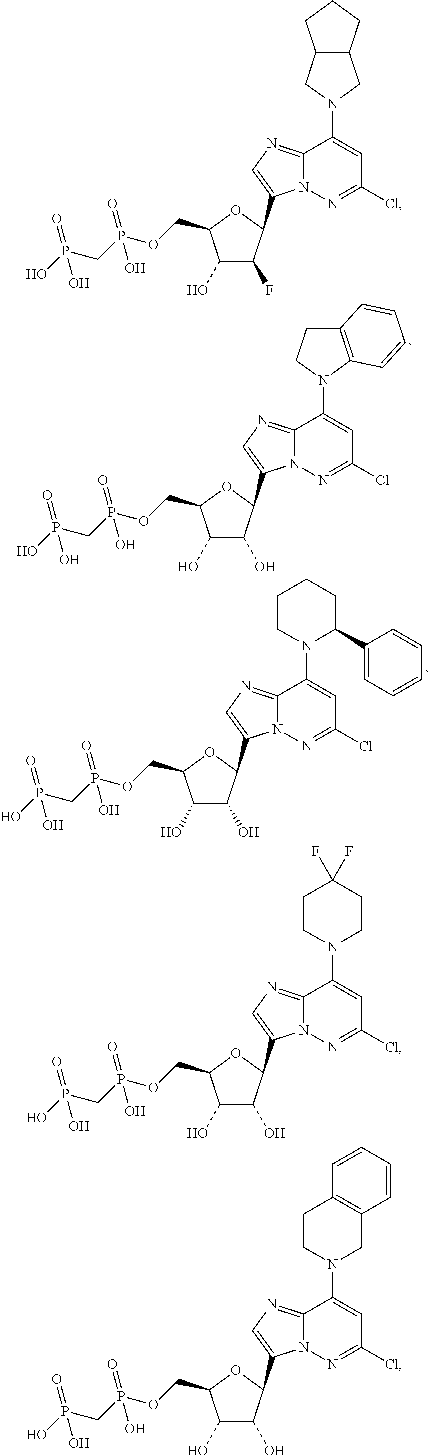 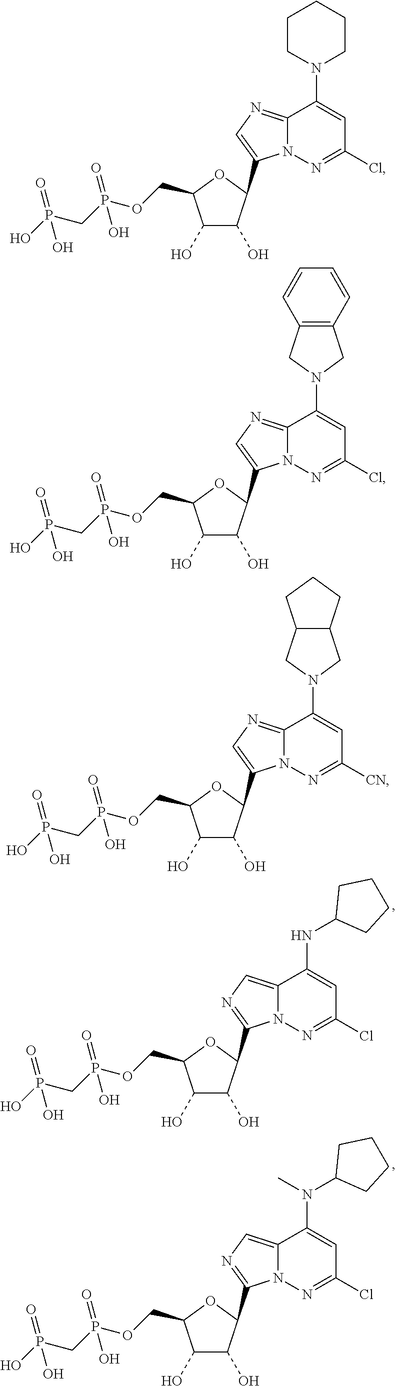 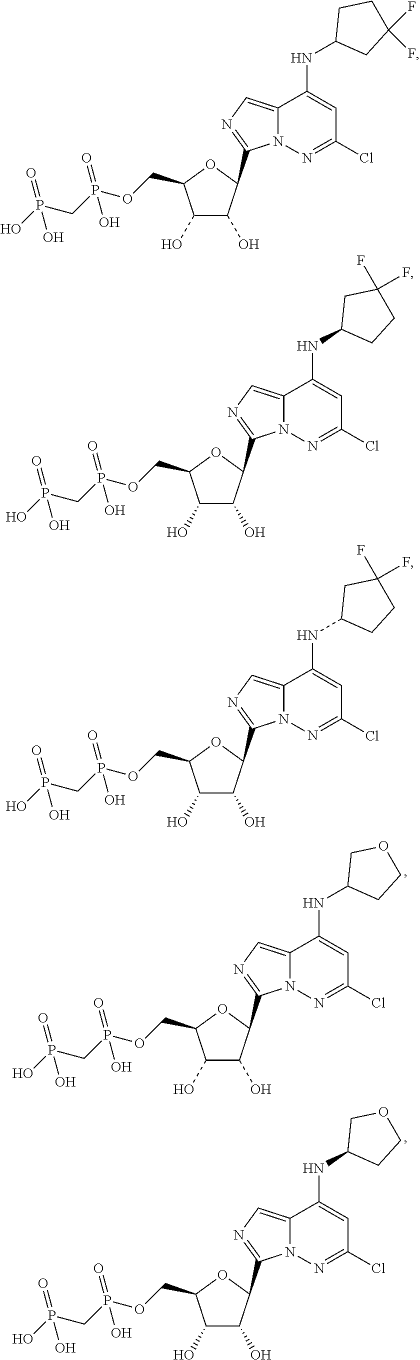 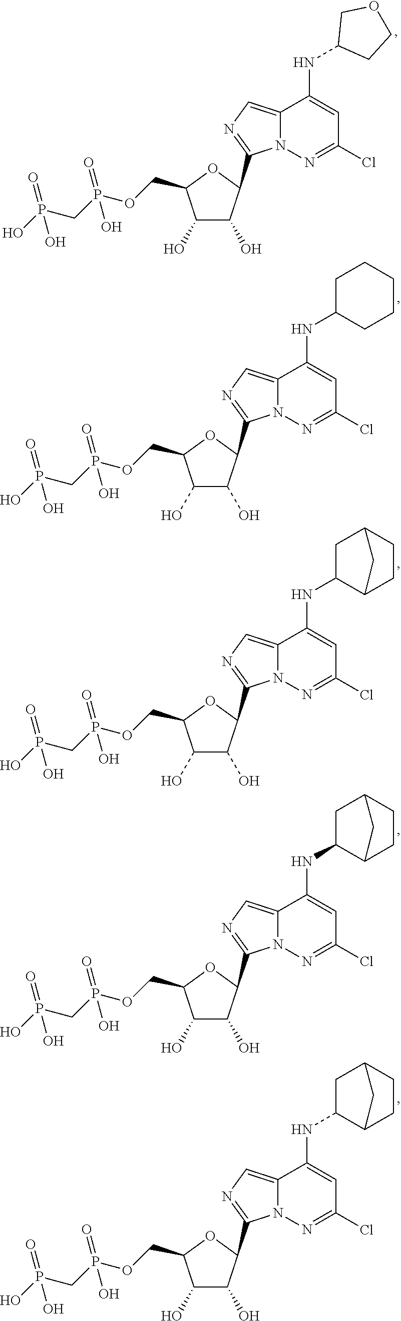 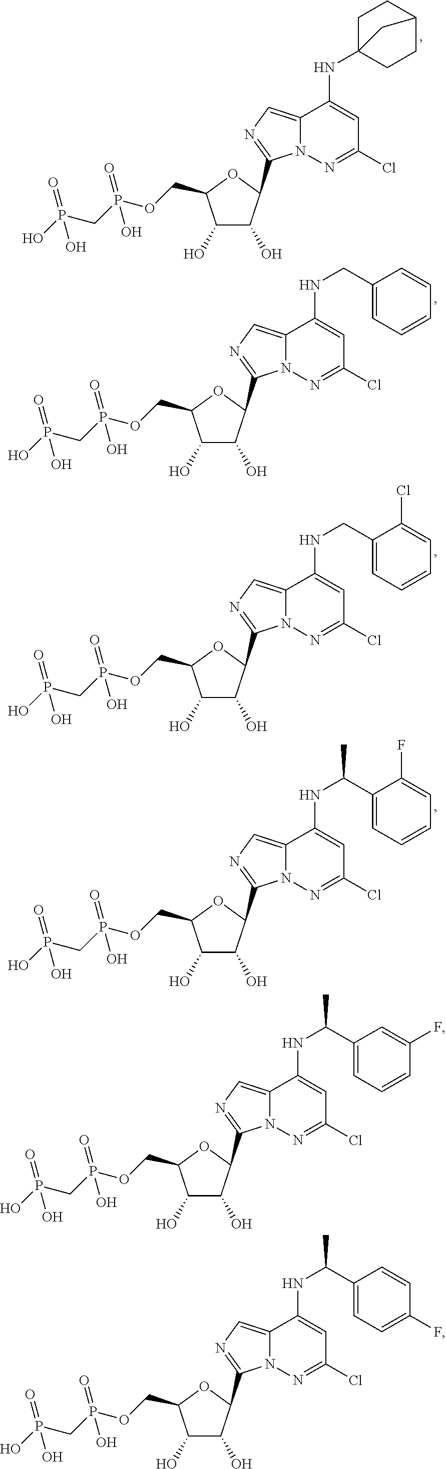 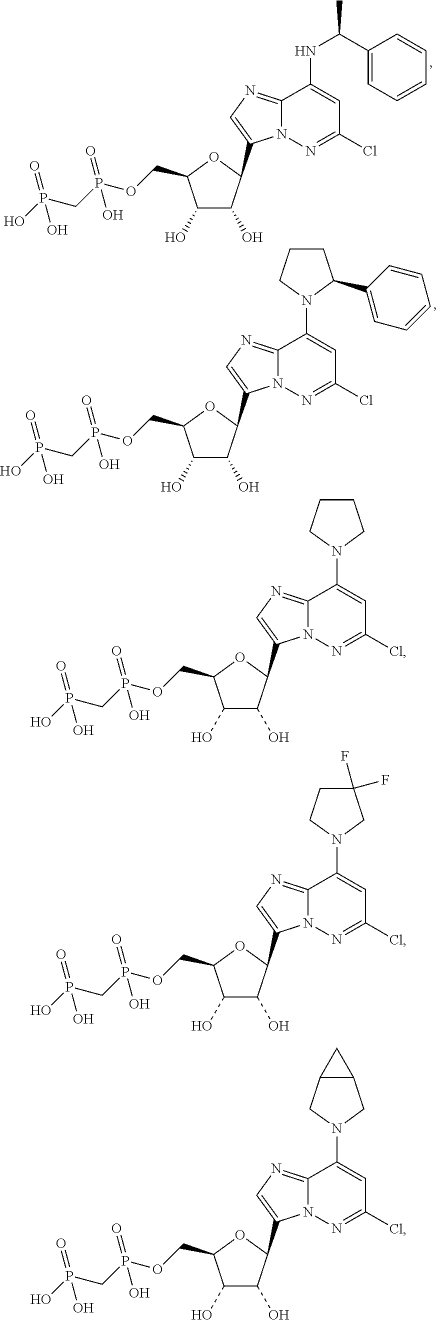 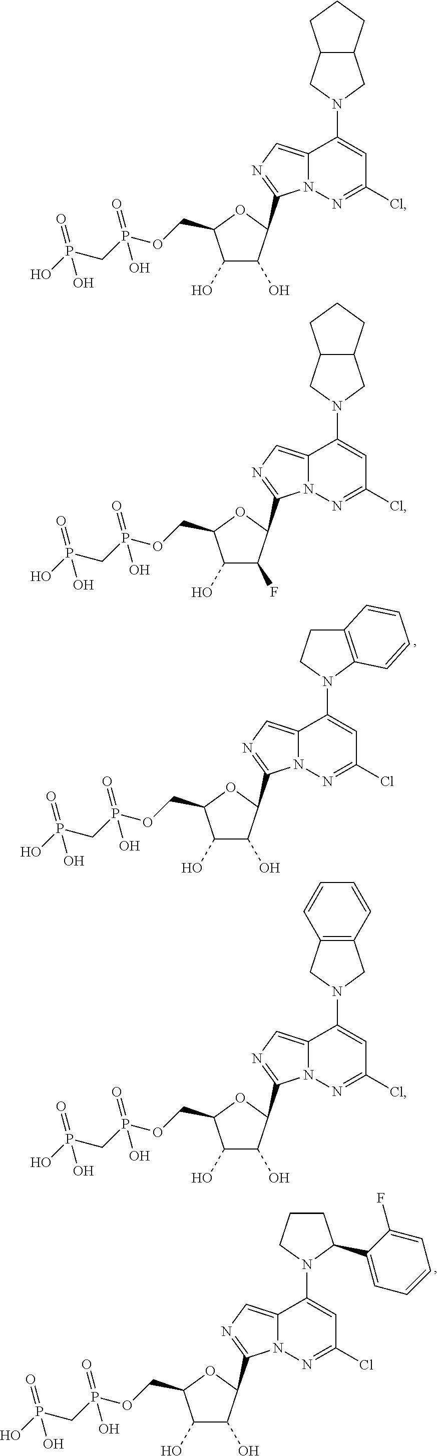 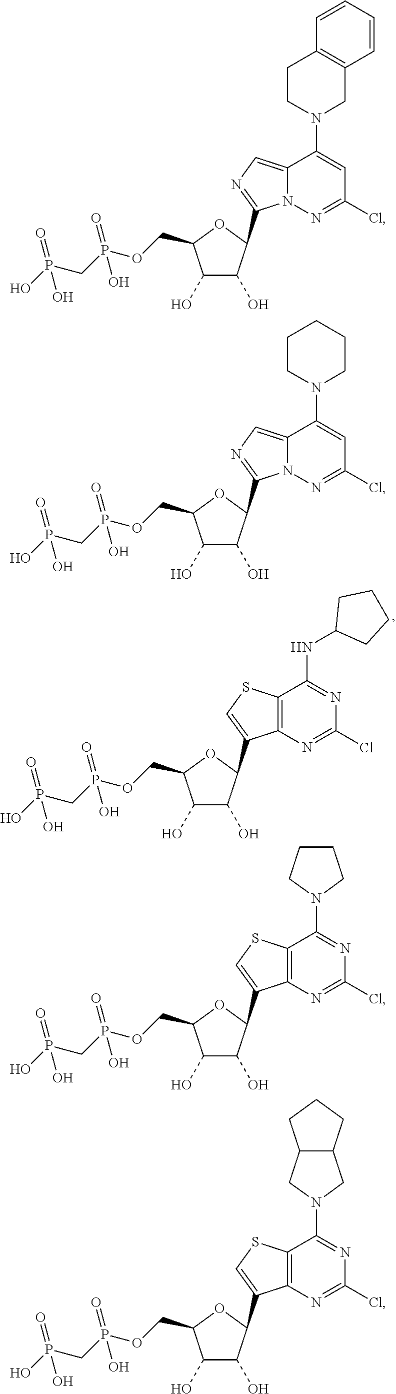 [SMILES: O=P(O)(O)CP(=O)(O)OC[C@H]1O[C@@H](c2ncc3c(N4CCc5ccccc5C4)cc(Cl)nn23)[C@H](O)[C@@H]1O ; O=P(O)(O)CP(=O)(O)OC[C@H]1O[C@@H](c2ncc3c(N4CCCCC4)cc(Cl)nn23)[C@H](O)[C@@H]1O ; O=P(O)(O)CP(=O)(O)OC[C@H]1O[C@@H](c2csc3c(NC4CCCC4)nc(Cl)nc23)[C@H](O)[C@@H]1O ; O=P(O)(O)CP(=O)(O)OC[C@H]1O[C@@H](c2csc3c(N4CCCC4)nc(Cl)nc23)[C@H](O)[C@@H]1O ; O=P(O)(O)CP(=O)(O)OC[C@H]1O[C@@H](c2csc3c(N4CC5CCCC5C4)nc(Cl)nc23)[C@H](O)[C@@H]1O] 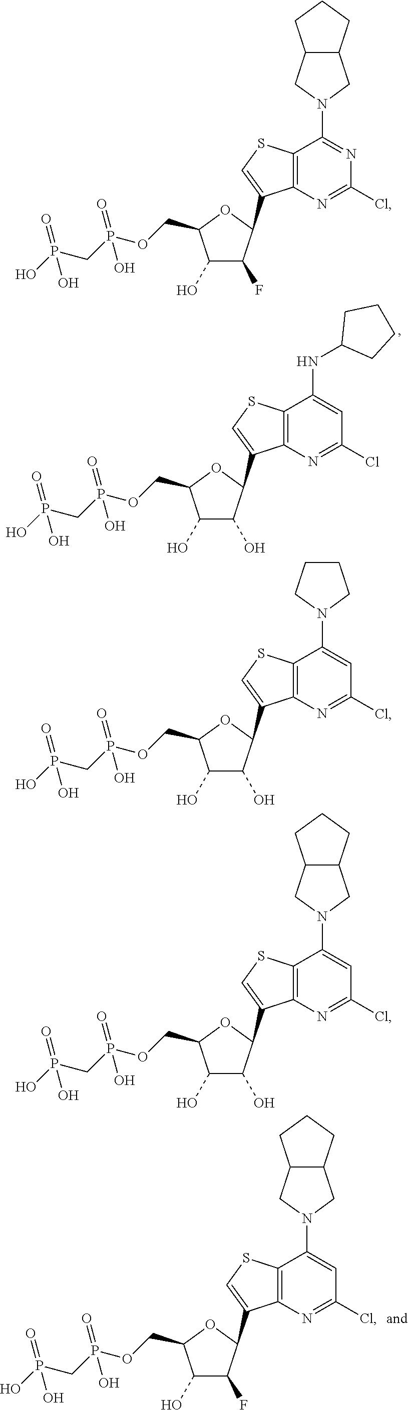 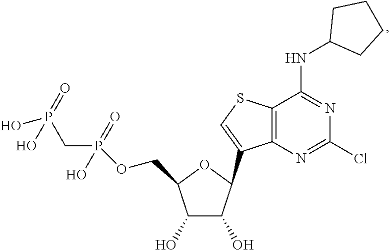 [SMILES: O=P(O)(O)CP(=O)(O)OC[C@H]1O[C@@H](c2csc3c(NC4CCCC4)nc(Cl)nc23)[C@H](O)[C@@H]1O]
or a stereoisomer, tautomer, prodrug, or a pharmaceutically acceptable salt of any of the foregoing.

33. A pharmaceutical composition comprising the compound of claim 1, or a stereoisomer, tautomer, prodrug, or a pharmaceutically acceptable salt of any of the foregoing, and a pharmaceutically acceptable excipient.

34. A kit comprising the compound of claim 1, or a stereoisomer, tautomer, prodrug, or a pharmaceutically acceptable salt of any of the foregoing.

35. A method of treating a disease mediated by CD73 in an individual in need thereof, comprising administering to the individual a therapeutically effective amount of the compound of claim 1, or a stereoisomer, tautomer, prodrug, or a pharmaceutically acceptable salt of any of the foregoing.

36. The method of claim 35, wherein the disease is cancer.

37. The method of claim 35, further comprising administering to the individual an additional therapeutic agent, wherein the additional therapeutic agent is an immune checkpoint inhibitor, a chemotherapeutic agent, an immune-modulating agent, an inflammation-modulating agent, or an anti-infective agent.

38. The method of claim 37, wherein the additional therapeutic agent is an immune checkpoint inhibitor.

39. The method of claim 38, wherein the additional therapeutic agent is a cytotoxic T lymphocyte associated protein 4 (CTLA-4) inhibitor, a programmed cell death protein 1 (PD-1) inhibitor, or a programmed death ligand 1 (PD-L1) inhibitor.

40. A method of reversing or stopping the progression of CD73-mediated immunosuppression in an individual, comprising administering to the individual a therapeutically effective amount of the compound of claim 1, or a stereoisomer, tautomer, prodrug, or a pharmaceutically acceptable salt of any of the foregoing.

41. A method of inhibiting CD73-catalyzed hydrolysis of adenosine monophosphate, comprising contacting CD73 with the compound of claim 1, or a stereoisomer, tautomer, prodrug, or a pharmaceutically acceptable salt of any of the foregoing.

42. (canceled)
